SEQUENCE LISTING (54 sequences)

SEQ ID NO: 1 - DNA, 372 bp, Artificial Sequence
  Synthetic variable heavy chain antibody NI-101.11
gaggtgcagctggtgcagtctgggggaggcgtggtccagcctgggaggtccctgagactctcctgtgcagcgtctggattcgccttcagtagctatggcatgcactgggtccgccaggctccaggcaaggggctggagtgggtggcagttatatggtttgatggaactaaaaaatactatacagactccgtgaagggcagattcaccatctccagagacaattccaagaacacactgtatctgcaaatgaacaccctgagagccgaggacacggctgtgtattactgtgcgagagataggggtataggagctcggcgggggccgtactacatggacgtctggggcaaagggaccacggtcaccgtctcctca

SEQ ID NO: 2 - DNA, 327 bp, Artificial Sequence
  Synthetic variable light chain antibody NI-101.13B
gacatccagttgacccagtctccttccaccctgtctgcatctgtaggagacagagtcaccatcacttgccgggccagtcagagtattagtagctggttggcctggtatcagcagattccagggaaagcccctaagctcctgatctataaggcgtctagtttagaaagtggggtcccatcaaggttcagcggcagtggatctgggacagaattcactctcaccatcagcagcctgcagcctgatgattttgcaacttattactgccaacagtataatagttattctcgaacgttcggccaagggaccaagctggagatcaaacgtacg

SEQ ID NO: 3 - DNA, 372 bp, Artificial Sequence
  Synthetic variable heavy chain antibody NI-101.10
gaggtgcagctagtgcagtctgggggaggcgtggtccagcctgggaggtccctgagactctcctgtgcagcgtctggattcgccttcagtagctatggcatacactgggtccgccaggctccaggcaaggggctggagtgggtggcagttatatggtttgatggaactaaaaaatactatacagactccgtgaagggcagattcaccatctccagagacaattccaagaacacactgtatctgcaaatgaacaccctgagagccgaggacacggctgtgtattactgtgcgagagataggggtataggagctcggcgggggccgtactacatggacgtctggggcaaagggaccacggtcaccgtctcctca

SEQ ID NO: 4 - protein, 124 aa, Artificial Sequence
  Synthetic variable heavy chain antibody NI-101.10
EVQLVQSGGGVVQPGRSLRLSCAASGFAFSSYGIHWVRQAPGKGLEWVAVIWFDGTKKYYTDSVKGRFTISRDNSKNTLYLQMNTLRAEDTAVYYCARDRGIGARRGPYYMDVWGKGTTVTVSS

SEQ ID NO: 5 - DNA, 372 bp, Artificial Sequence
  Synthetic variable heavy chain antibody NI-101.11
gaggtgcagctggtgcagagcggcggcggcgtggtgcagcccggccggagcctgcggctgagctgcgccgccagcggcttcgccttcagcagctacggcatgcactgggtgcggcaggcccccggcaagggcctggagtgggtggccgtgatctggttcgacggcaccaagaagtactacaccgacagcgtgaagggccggttcaccatcagccgggacaacagcaagaacaccctgtacctgcagatgaacaccctgcgggccgaggacaccgccgtgtactactgcgcccgggaccggggcatcggcgcccggcggggcccctactacatggacgtgtggggcaagggcaccaccgtgaccgtgagcagc

SEQ ID NO: 6 - protein, 124 aa, Artificial Sequence
  Synthetic variable heavy chain antibody NI-101.11
EVQLVQSGGGVVQPGRSLRLSCAASGFAFSSYGMHWVRQAPGKGLEWVAVIWFDGTKKYYTDSVKGRFTISRDNSKNTLYLQMNTLRAEDTAVYYCARDRGIGARRGPYYMDVWGKGTTVTVSS

SEQ ID NO: 7 - DNA, 327 bp, Artificial Sequence
  Synthetic variable light chain antibody NI-101.10 and NI-101.11
gaaattgtgctgactcagtctccatcctccctgtctgcatctgtaggagacagagtcaccatcacttgccgggcaagtcagagcattagcagctatttaaattggtatcaacagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttattactgtcagcagagttacagtacccctctcactttcggcggagggaccaagctcgagatcaaacgtacg

SEQ ID NO: 8 - protein, 108 aa, Artificial Sequence
  Synthetic variable light chain antibody NI-101.10 and NI-101.11
EIVLTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKLEIKR

SEQ ID NO: 9 - DNA, 381 bp, Artificial Sequence
  Synthetic variable heavy chain antibody NI-101.12
gaggtgcagctggtggagagcggccccggcctggtgaagcccgccgagaccctgagcctgacctgcaccgtgagcggcggcagcatccggagcggcagcatctgctggtactggatccggcagccccccggcaagggcctggagtggatcggctacttctgctacagcggcgccaccttctacacccccagcctgcggggccggctgaccatcagcgtggacgccagcaagaaccagctgagcctgagcctgagcagcgtgaccgccgccgacaccgccgtgtactactgcgcccggcgggccggcgagaacagcggcggcatcgagccctactacggcatggacgtgtggggccagggcaccaccgtgaccgtgagcagc

SEQ ID NO: 10 - protein, 127 aa, Artificial Sequence
  Synthetic variable heavy chain antibody NI-101.12
EVQLVESGPGLVKPAETLSLTCTVSGGSIRSGSICWYWIRQPPGKGLEWIGYFCYSGATFYTPSLRGRLTISVDASKNQLSLSLSSVTAADTAVYYCARRAGENSGGIEPYYGMDVWGQGTTVTVSS

SEQ ID NO: 11 - DNA, 330 bp, Artificial Sequence
  Synthetic variable light chain antibody NI-101.12
gacgagatcgtgctgacccagagccccagcagcctgagcgccagcatcggcgaccgggtgaccatcacctgccgggccagcgagagcatcaacaagtacgtgaactggtaccagcagaagcccggcaaggcccccaagctgctgatctacgccgccagcagcctgcagagcggcgcccccagccgggtgagcggcagcggcttcggccgggacttcagcctgaccatcagcggcctgcaggccgaggacttcggcgcctacttctgccagcagagctacagcgccccctacaccttcggccagggcaccaaggtggagatcaagcggacc

SEQ ID NO: 12 - protein, 110 aa, Artificial Sequence
  Synthetic variable light chain antibody NI-101.12
DEIVLTQSPSSLSASIGDRVTITCRASESINKYVNWYQQKPGKAPKLLIYAASSLQSGAPSRVSGSGFGRDFSLTISGLQAEDFGAYFCQQSYSAPYTFGQGTKVEIKRT

SEQ ID NO: 13 - DNA, 363 bp, Artificial Sequence
  Synthetic variable heavy chain antibody NI-101.13
caggtacagctgcaggagtcaggcccaggactggtgaagccttcggagaccctgtccctcacctgcactgtctctggtggctccatcagcagaagaagttactactggggctggatccgccagtccccagggaaggggctggagtggagtggaagtatccattatagcgggagcacctactacaacccgtccctcaagagtcgagtcaccatatctgtagacacgtccaagaaccagttctccctgaaactgagctctgttaccgccgcagacacggctgtctattactgtgcgagatcacgttggggcagcagctgggtatttgactactggggccagggcacactggtcaccgtctcttcg

SEQ ID NO: 14 - protein, 121 aa, Artificial Sequence
  Synthetic variable heavy chain antibody NI-101.13
QVQLQESGPGLVKPSETLSLTCTVSGGSISRRSYYWGWIRQSPGKGLEWSGSIHYSGSTYYNPSLKSRVTISVDTSKNQFSLKLSSVTAADTAVYYCARSRWGSSWVFDYWGQGTLVTVSS

SEQ ID NO: 15 - DNA, 330 bp, Artificial Sequence
  Synthetic variable light chain antibody NI-101.13
cagagcgtgctgacccagccgccgagcgcgagcggcaccccgggccagcgcgtgaccattagctgcagcggcagcagcagcaacattggcagcaactatgtgtattggtatcagcagccgccgggcaccgcgccgaaactgctgatttatcgcaacaaccagcgcccgagcggcgtgccggatcgctttagcggcagcaaaagcggcaccagcgcgagcctggcgattagcggcctgcgcagcgaagatgaagcggattattattgcgcggcgtgggatgatagcctgagcggctatgtgtttggcaccggcaccaaagtgaccgtgctg

SEQ ID NO: 16 - protein, 111 aa, Artificial Sequence
  Synthetic variable light chain antibody NI-101.13
QSVLTQPPSASGTPGQRVTISCSGSSSNIGSNYVYWYQQPPGTAPKLLIYRNNQRPSGVPDRFSGSKSGTSASLAISGLRSEDEADYYCAAWDDSLSGYVFGTGTKVTVLG

SEQ ID NO: 17 - protein, 5 aa, Artificial Sequence
  Synthetic variable heavy chain antibody NI-101.10 CDR1
SYGIH

SEQ ID NO: 18 - protein, 17 aa, Artificial Sequence
  Synthetic variable heavy chain antibody NI-101.10 CDR2
VIWFDGTKKYYTDSVKG

SEQ ID NO: 19 - protein, 15 aa, Artificial Sequence
  Synthetic variable heavy chain antibody NI-101.10 CDR3
DRGIGARRGPYYMDV

SEQ ID NO: 20 - protein, 5 aa, Artificial Sequence
  Synthetic variable heavy chain antibody NI-101.11 and NI-101.12F6A CDR1
SYGMH

SEQ ID NO: 21 - protein, 17 aa, Artificial Sequence
  Synthetic variable heavy chain antibody NI-101.11 and NI-101.12F6A CDR2
VIWFDGTKKYYTDSVKG

SEQ ID NO: 22 - protein, 15 aa, Artificial Sequence
  Synthetic variable heavy chain antibody NI-101.11 and NI-101.12F6A CDR3
DRGIGARRGPYYMDV

SEQ ID NO: 23 - protein, 11 aa, Artificial Sequence
  Synthetic variable light chain antibody NI-101.10, NI-101.11, and NI-101.12F6A CDR1
RASQSISSYLN

SEQ ID NO: 24 - protein, 7 aa, Artificial Sequence
  Synthetic variable light chain antibody NI-101.10, NI-101.11, and NI-101.12F6A CDR2
AASSLQS

SEQ ID NO: 25 - protein, 9 aa, Artificial Sequence
  Synthetic variable light chain antibody NI-101.10, NI-101.11, and NI-101.12F6A CDR3
QQSYSTPLT

SEQ ID NO: 26 - protein, 5 aa, Artificial Sequence
  Synthetic variable heavy chain antibody NI-101.12 CDR1
SGSIC

SEQ ID NO: 27 - protein, 19 aa, Artificial Sequence
  Synthetic variable heavy chain antibody NI-101.12 CDR2
WIGYFCYSGATFYTPSLRG

SEQ ID NO: 28 - protein, 17 aa, Artificial Sequence
  Synthetic variable heavy chain antibody NI-101.12 CDR3
RAGENSGGIEPYYGMDV

SEQ ID NO: 29 - protein, 11 aa, Artificial Sequence
  Synthetic variable light chain antibody NI-101.12 CDR1
RASESINKYVN

SEQ ID NO: 30 - protein, 7 aa, Artificial Sequence
  Synthetic variable light chain antibody NI-101.12 CDR2
AASSLQS

SEQ ID NO: 31 - protein, 9 aa, Artificial Sequence
  Synthetic variable light chain antibody NI-101.12 CDR3
QQSYSAPYT

SEQ ID NO: 32 - protein, 7 aa, Artificial Sequence
  Synthetic variable heavy chain antibody NI-101.13, NI-101.13A, and NI-101.13B CDR1
RRSYYWG

SEQ ID NO: 33 - protein, 16 aa, Artificial Sequence
  Synthetic variable heavy chain antibody NI-101.13, NI-101.13A, and NI-101.13B CDR2
SIHYSGSTYYNPSLKS

SEQ ID NO: 34 - protein, 11 aa, Artificial Sequence
  Synthetic variable heavy chain antibody NI-101.13, NI-101.13A, and NI-101.13B CDR3
SRWGSSWVFDY

SEQ ID NO: 35 - protein, 13 aa, Artificial Sequence
  Synthetic variable light chain antibody NI-101.13 CDR1
SGSSSNIGSNYVY

SEQ ID NO: 36 - protein, 7 aa, Artificial Sequence
  Synthetic variable light chain antibody NI-101.13 CDR2
RNNQRPS

SEQ ID NO: 37 - protein, 11 aa, Artificial Sequence
  Synthetic variable light chain antibody NI-101.13 CDR3
AAWDDSLSGYV

SEQ ID NO: 38 - DNA, 372 bp, Artificial Sequence
  Synthetic variable heavy chain antibody NI-101.12F 6A
caggtgcagctggtggagtctgggggaggcgtggtccagcctgggaggtccctgagactctcctgtgcagcgtctggattcgccttcagtagctatggcatgcactgggtccgccaggctccaggcaaggggctggagtgggtggcagttatatggtttgatggaactaaaaaatactatacagactccgtgaagggcagattcaccatctccagagacaattccaagaacacactgtatctgcaaatgaacaccctgagagccgaggacacggctgtgtattactgtgcgagagataggggtataggagctcggcgggggccgtactacatggacgtctggggcaaagggaccacggtcaccgtctcctca

SEQ ID NO: 39 - protein, 124 aa, Artificial Sequence
  Synthetic variable heavy chain antibody NI-101.12F 6A
QVQLVESGGGVVQPGRSLRLSCAASGFAFSSYGMHWVRQAPGKGLEWVAVIWFDGTKKYYTDSVKGRFTISRDNSKNTLYLQMNTLRAEDTAVYYCARDRGIGARRGPYYMDVWGKGTTVTVSS

SEQ ID NO: 40 - DNA, 324 bp, Artificial Sequence
  Synthetic variable light chain antibody NI-101.12F 6A
gacatccagatgacccagtctccatcctccctgtctgcatctgtaggagacagagtcaccatcacttgccgggcaagtcagagcattagcagctatttaaattggtatcaacagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttattactgtcagcagagttacagtacccctctcactttcggcggagggaccaaggtggagatcaaacgt

SEQ ID NO: 41 - protein, 108 aa, Artificial Sequence
  Synthetic variable light chain antibody NI-101.12F 6A
DIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIKR

SEQ ID NO: 42 - protein, 121 aa, Artificial Sequence; identical to SEQ ID NO: 14
  Synthetic variable heavy chain antibody NI-101.13A

SEQ ID NO: 43 - protein, 121 aa, Artificial Sequence; identical to SEQ ID NO: 14
  Synthetic variable heavy chain antibody NI-101.13B

SEQ ID NO: 44 - protein, 107 aa, Artificial Sequence
  Synthetic variable light chain antibody NI-101.13A
DIQLTQSPSSLSASVGDRVTITCRASQSISSYLNWYQQKPGKAPKLLIYAASSLQSGVPSRFSGSGSGTDFTLTISSLQPEDFATYYCQQSYSTRTFGQGTKVEIKR

SEQ ID NO: 45 - protein, 108 aa, Artificial Sequence
  Synthetic variable light chain antibody NI-101.13B
DIQLTQSPSTLSASVGDRVTITCRASQSISSWLAWYQQIPGKAPKLLIYKASSLESGVPSRFSGSGSGTEFTLTISSLQPDDFATYYCQQYNSYSRTFGQGTKLEIKR

SEQ ID NO: 46 - protein, 11 aa, Artificial Sequence
  Synthetic variable light chain antibody NI-101.13A CDR1
RASQSISSYLN

SEQ ID NO: 47 - protein, 7 aa, Artificial Sequence
  Synthetic variable light chain antibody NI-101.13A CDR2
AASSLQS

SEQ ID NO: 48 - protein, 8 aa, Artificial Sequence
  Synthetic variable light chain antibody NI-101.13A CDR3
QQSYSTRT

SEQ ID NO: 49 - protein, 11 aa, Artificial Sequence
  Synthetic variable light chain antibody NI-101.13B CDR1
RASQSISSWLA

SEQ ID NO: 50 - protein, 7 aa, Artificial Sequence
  Synthetic variable light chain antibody NI-101.13B CDR2
KASSLES

SEQ ID NO: 51 - protein, 9 aa, Artificial Sequence
  Synthetic variable light chain antibody NI-101.13B CDR3
QQYNSYSRT

SEQ ID NO: 52 - DNA, 363 bp, Artificial Sequence
  Synthetic variable heavy chain antibody NI-101.13A
caggtgcagctgcaggagtcgggcccaggactggtgaagccttcggagaccctgtccctcacctgcactgtctctggtggctccatcagcagaagaagttactactggggctggatccgccagtccccagggaaggggctggagtggagtggaagtatccattatagcgggagcacctactacaacccgtccctcaagagtcgagtcaccatatctgtagacacgtccaagaaccagttctccctgaaactgagctctgttaccgccgcagacacggctgtctattactgtgcgagatcacgttggggcagcagctgggtatttgactactggggccagggaaccctggtcaccgtctcctcg

SEQ ID NO: 53 - DNA, 363 bp, Artificial Sequence; identical to SEQ ID NO: 52
  Synthetic variable heavy chain antibody NI-101.13B

SEQ ID NO: 54 - DNA, 324 bp, Artificial Sequence
  Synthetic variable light chain antibody NI-101.13A
gacatccagttgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcaacagagttacagtaccagaacgttcggccaagggaccaaggtggagatcaaacgtacg

(19) United States
(12) Patent Application Publication
(10) Pub. No.: US 2011/0182809 A1
(43) Pub. Date: Jul. 28, 2011

(54) Method of Promoting Neurogenesis

(75) Inventors: Roger Nitsch, Zumikon (CH); Olle Lindvall, Lund (SE); Barbara Biscaro, Zurich (CH); Christine Ekdahl, Lund (SE)

(73) Assignee: University of Zurich, Zurich (CH)

(21) Appl. No.: 13/003,245

(22) PCT Filed: Jul. 9, 2009
(86) PCT No.: PCT/IB09/06666

§ 371 Date: Apr. 4, 2011

Related U.S. Application Data
(60) Provisional application No. 61/079,379, filed on Jul. 9, 2008.

(51) Int. Cl. A61K 51/10; A61K 39/395; A61P 25/28; A61P 25/00; A61P 25/16

(52) U.S. Cl. 424/1.49; 424/130.1

(57) ABSTRACT

The present description relates generally to methods of using Abeta binding molecules including, for example, antibodies and antibody fragments that recognize Abeta. The description provides methods of promoting neurogenesis, angiogenesis, synaptic activity and/or dendritic arborization using Abeta binding molecules. The description also provides methods of treating various diseases, disorder, injuries and conditions associated with amyloid plaques or the accumulation of Abeta.

### FIELD OF THE INVENTION

The present description relates generally to the fields of neurology, neurobiology and molecular biology. This description relates to methods of using Abeta binding molecules.

### BACKGROUND OF THE INVENTION

Neurodegenerative disease is an important concern. Neural damage as a result of stroke or trauma to the brain, as well as neurodegenerative diseases such as Alzheimer's disease, is a leading cause of death and disability. The failure of the mammalian nervous system to completely regenerate after injury is a major clinical problem. While several methods for in vivo detection of Alzheimer's and related diseases have been reported (See e.g., Ruy and Chen, Front. Biosci. 13:777-89 (2008)), no marketed drug is known to promote neurogenesis and regeneration of neural tissues. Thus, it would be advantageous to provide a solution to the long-felt unmet medical need for therapeutic means of neuroregeneration.

### SUMMARY OF THE INVENTION

Some embodiments described herein provide a method of promoting neurogenesis, the method comprising administering to a subject an effective amount of an Abeta binding molecule.

Some embodiments described herein provide a method of promoting angiogenesis, the method comprising administering to a subject an effective amount of an Abeta binding molecule.

Some embodiments described herein provide a method of promoting synaptic density and/or activity, the method comprising administering to a subject an effective amount of an Abeta binding molecule.

Some embodiments described herein provide a method of promoting the dendritic arborization of CNS neurons in a subject, the method comprising administering to a subject an effective amount of an Abeta binding molecule. In one embodiment, the CNS neurons are granular neurons.

In further embodiments of the methods described herein, the subject has an accumulation of Abeta.

In some embodiments, the description herein provides a method of treating an abnormal amyloid condition, the method comprising administering to a subject in need thereof a therapeutically effective amount of an Abeta binding molecule, wherein the Abeta binding molecule promotes neurogenesis.

In some embodiments, the Abeta binding molecule specifically binds a peptide selected from the group consisting of Abeta₁₋₄₂peptide, Abeta₁₋₄₀ peptide, and Abeta₁₋₄₃peptide. In some embodiments, the Abeta binding molecule specifically binds fibrillar Abeta or beta-amyloid fibrils. In some embodiments, the Abeta binding molecule can specifically bind diffuse beta-amyloid deposits. In some embodiments, the Abeta binding molecule can specifically bind a neoepitope of Abeta. In some embodiments, the Abeta binding molecule can specifically bind a beta-amyloid plaque. In still further embodiments, the Abeta binding molecule specifically binds an Abeta species selected from the group consisting of N-terminally truncated Abeta species, C-terminally truncated Abeta species, pyroglutamate-modified Abeta species, redox-modified Abeta species, and dimeric Abeta species.

In various embodiments of the above-described methods, the Abeta binding molecule is an anti-Abeta antibody or antigen-binding fragment thereof. In some embodiments, the heavy chain variable region (VH) framework regions of the anti-Abeta antibody or antigen-binding fragment thereof are human, except for five or fewer amino acid substitutions. In some embodiments, the light chain variable region (VL) framework regions of the anti-Abeta antibody or antigen-binding fragment thereof are human, except for five or fewer amino acid substitutions. In some embodiments, the heavy and light chain variable regions are fully human.

In various embodiments of the above-described methods, the anti-Abeta antibody or antigen-binding fragment thereof is fully human.

In some embodiments, the heavy chain variable region (VH) framework regions of the anti-Abeta antibody or antigen-binding fragment thereof are murine, except for five or fewer amino acid substitutions. In some embodiments, the light chain variable region (VL) framework regions of the anti-Abeta antibody or antigen-binding fragment thereof are murine, except for five or fewer amino acid substitutions.

In various embodiments of the above-described methods, the anti-Abeta antibody or antigen-binding fragment thereof is humanized.

In various embodiments of the above-described methods, the anti-Abeta antibody or antigen-binding fragment thereof is chimeric.

In various embodiments of the above-described methods, the anti-Abeta antibody or antigen-binding fragment thereof is primatized.

In certain embodiments of the above-described methods, the antibodies or fragments thereof are Fab fragments, Fab′ fragments, F(ab)₂ fragments, or Fv fragments.

In certain embodiments of the above-described methods, the antibodies or fragments thereof are single chain antibodies.

In certain embodiments of the above-described methods, the antibodies or fragments thereof are multivalent, and comprises at least two heavy chains and at least two light chains. In certain embodiments of the above-described methods, the antibodies or fragments thereof are multispecific. In certain embodiments of the above-described methods, the antibodies or fragments thereof are bispecific.

In some embodiments, the methods described provide an antibody where the heavy chain variable region (VH) of the anti-Abeta antibody or antigen-binding fragment thereof comprises an amino acid sequence at least 90% identical to a reference amino acid sequence selected from the group consisting of: SEQ ID NO: 4, SEQ ID NO: 6, SEQ ID NO: 10, SEQ ID NO: 14, SEQ ID NO: 39, SEQ ID NO: 42, and SEQ ID NO: 43. In one embodiment, the VH of the anti-Abeta antibody or antigen-binding fragment thereof comprises an amino acid sequence selected from the group consisting of: SEQ ID NO: 4, SEQ ID NO: 6, SEQ ID NO: 10, SEQ ID NO: 14, SEQ ID NO: 39, SEQ ID NO: 42, and SEQ ID NO: 43.

In some embodiments, the methods described provide an antibody where the light chain variable region (VL) of the anti-Abeta antibody or antigen-binding fragment thereof comprises an amino acid sequence at least 90% identical to a reference amino acid sequence selected from the group consisting of: SEQ ID NO: 8, SEQ ID NO: 12, SEQ ID NO: 16, SEQ ID NO: 41, SEQ ID NO: 44, and SEQ ID NO:45. In one embodiment, the VL of the anti-Abeta antibody or antigen-binding fragment thereof comprises an amino acid sequence selected from the group consisting of: SEQ ID NO: 8, SEQ ID NO: 12, SEQ ID NO: 16, SEQ ID NO: 41, SEQ ID NO: 44, and SEQ ID NO:45.

In another embodiment, the methods described provide an antibody where the heavy chain variable region (VH) of the anti-Abeta antibody or antigen-binding fragment thereof comprises an amino acid sequence identical, except for 20 or fewer conservative amino acid substitutions, to a reference amino acid sequence selected from the group consisting of: SEQ ID NO: 4, SEQ ID NO: 6, SEQ ID NO: 10, SEQ ID NO: 14, SEQ ID NO: 39, SEQ ID NO: 42, and SEQ ID NO: 43.

In yet another embodiment, the methods described provide an antibody where the light chain variable region (VL) of the anti-Abeta antibody or antigen-binding fragment thereof comprises an amino acid sequence identical, except for 20 or fewer conservative amino acid substitutions, to a reference amino acid sequence selected from the group consisting of: SEQ ID NO: 8, SEQ ID NO: 12, SEQ ID NO: 16, SEQ ID NO: 41, SEQ ID NO: 44, and SEQ ID NO:45.

In some embodiments, the methods described herein provide an antibody where the heavy chain variable region (VH) and the light chain variable region (VL) of the anti-Abeta antibody or antigen-binding fragment thereof comprise, respectively, amino acid sequences at least 90% identical to reference amino acid sequences selected from the group consisting of: SEQ ID NO: 4 and SEQ ID NO: 8; SEQ ID NO: 6 and SEQ ID NO: 8; SEQ ID NO: 10 and SEQ ID NO: 12; SEQ ID NO: 14 and SEQ ID NO: 16; SEQ ID NO: 39 and SEQ ID NO: 41; SEQ ID NO: 42 and SEQ ID NO: 44; and SEQ ID NO: 43 and SEQ ID NO: 45.

In a further embodiment, the methods described provide an antibody where the heavy chain variable region (VH) and the light chain variable region (VL) of the anti-Abeta antibody or antigen-binding fragment thereof comprise, respectively, amino acid sequences selected from the group consisting of: SEQ ID NO: 4 and SEQ ID NO: 8; SEQ ID NO: 6 and SEQ ID NO: 8; SEQ ID NO: 10 and SEQ ID NO: 12; SEQ ID NO: 14 and SEQ ID NO: 16; SEQ ID NO: 39 and SEQ ID NO: 41; SEQ ID NO: 42 and SEQ ID NO: 44; and SEQ ID NO: 43 and SEQ ID NO: 45.

In other embodiments, the methods described herein provide an antibody where the heavy chain variable region (VH) and the light chain variable region (VL) of the anti-Abeta antibody or antigen-binding fragment thereof comprise, respectively, amino acid sequences identical, except for 20 or fewer conservative amino acid substitutions each, to reference amino acid sequences selected from the group consisting of: SEQ ID NO: 4 and SEQ ID NO: 8; SEQ ID NO: 6 and SEQ ID NO: 8; SEQ ID NO: 10 and SEQ ID NO: 12; SEQ ID NO: 14 and SEQ ID NO: 16; SEQ ID NO: 39 and SEQ ID NO: 41; SEQ ID NO: 42 and SEQ ID NO: 44; and SEQ ID NO: 43 and SEQ ID NO: 45.

In another embodiment, the methods provide an antibody where the heavy chain variable region (VH) of the anti-Abeta antibody or antigen-binding fragment thereof comprises a Kabat heavy chain complementarity determining region-1 (VH-CDR1) amino acid sequence identical, except for two or fewer amino acid substitutions, to a reference VH-CDR1 amino acid sequence selected from the group consisting of: SEQ ID NO: 17, SEQ ID NO: 20, SEQ ID NO: 26, and SEQ ID NO: 32. In one embodiment, the VH-CDR1 amino acid sequence is selected from the group consisting of: SEQ ID NO: 17, SEQ ID NO: 20, SEQ ID NO: 26, and SEQ ID NO: 32.

In some embodiments, the methods provide an antibody where the heavy chain variable region (VH) of the anti-Abeta antibody or antigen-binding fragment thereof comprises a Kabat heavy chain complementarity determining region-2 (VH-CDR2) amino acid sequence identical, except for four or fewer amino acid substitutions, to a reference VH-CDR2 amino acid sequence selected from the group consisting of: SEQ ID NO: 18, SEQ ID NO: 21, SEQ ID NO: 27, and SEQ ID NO: 33. In one embodiment, the VH-CDR2 amino acid sequence is selected from the group consisting of: SEQ ID NO: 18, SEQ ID NO: 21, SEQ ID NO: 27, and SEQ ID NO: 33.

In some embodiments, the methods provide an antibody where the heavy chain variable region (VH) of the anti-Abeta antibody or antigen-binding fragment thereof comprises a Kabat heavy chain complementarity determining region-3 (VH-CDR3) amino acid sequence identical, except for four or fewer amino acid substitutions, to a reference VH-CDR3 amino acid sequence selected from the group consisting of: SEQ ID NO: 19, SEQ ID NO: 22, SEQ ID NO: 28, and SEQ ID NO: 34. In one embodiment, the VH-CDR3 amino acid sequence is selected from the group consisting of: SEQ ID NO: 19, SEQ ID NO: 22, SEQ ID NO: 28, and SEQ ID NO: 34.

In certain embodiments, the methods provide an antibody where the light chain variable region (VL) of the anti-Abeta antibody or antigen-binding fragment thereof comprises a Kabat light chain complementarity determining region-1 (VL-CDR1) amino acid sequence identical, except for four or fewer amino acid substitutions, to a reference VL-CDR1 amino acid sequence selected from the group consisting of: SEQ ID NO: 23, SEQ ID NO: 29, SEQ ID NO: 35, SEQ ID NO: 46, and SEQ ID NO: 49. In one embodiment, the VL-CDR1 amino acid sequence is selected from the group consisting of: SEQ ID NO: 23, SEQ ID NO: 29, SEQ ID NO: 35, SEQ ID NO: 46, and SEQ ID NO: 49.

In other embodiments, the methods described herein provide an antibody where the light chain variable region (VL) of the anti-Abeta antibody or antigen-binding fragment thereof comprises a Kabat light chain complementarity determining region-2 (VL-CDR2) amino acid sequence identical, except for two or fewer amino acid substitutions, to a reference VL-CDR2 amino acid sequence selected from the group consisting of SEQ ID NO: 24, SEQ ID NO: 30, SEQ ID NO: 36, SEQ ID NO: 47, and SEQ ID NO: 50. In one embodiment, the VL-CDR2 amino acid sequence is selected from the group consisting of: SEQ ID NO: 24, SEQ ID NO: 30, SEQ ID NO: 36, SEQ ID NO: 47, and SEQ ID NO: 50.

In other embodiments, the methods described herein provide an antibody where the light chain variable region (VL) of the anti-Abeta antibody or antigen-binding fragment thereof comprises a Kabat light chain complementarity determining region-3 (VL-CDR3) amino acid sequence identical, except for four or fewer amino acid substitutions, to a reference VL-CDR3 amino acid sequence selected from the group consisting of: SEQ ID NO: 25, SEQ ID NO: 31, SEQ ID NO: 37, SEQ ID NO: 48, and SEQ ID NO: 51. In one embodiment, the VL-CDR3 amino acid sequence is selected from the group consisting of SEQ ID NO: 25, SEQ ID NO: 31, SEQ ID NO: 37, SEQ ID NO: 48, and SEQ ID NO: 51.

In certain embodiments, the methods described herein provide an antibody where the heavy chain variable region (VH) of the anti-Abeta antibody or antigen-binding fragment thereof comprises VH-CDR1, VH-CDR2, and VH-CDR3 amino acid sequences selected from the group consisting of SEQ ID NOs: 17, 18, and 19; SEQ ID NOs: 20, 21, and 22; SEQ ID NOs: 26, 27, and 28; and SEQ ID NOs: 32, 33, and 34, except for one, two, three, or four amino acid substitutions in at least one of the VH-CDRs. In further embodiments, the VH of the anti-Abeta antibody or antigen-binding fragment thereof comprises VH-CDR1, VH-CDR2, and VH-CDR3 amino acid sequences selected from the group consisting of: SEQ ID NOs: 17, 18, and 19; SEQ ID NOs: 20, 21, and 22; SEQ ID NOs: 26, 27, and 28; and SEQ ID NOs: 32, 33, and 34.

In certain embodiments, the methods described herein provide an antibody where the light chain variable region (VL) of the anti-Abeta antibody or antigen-binding fragment thereof comprises VL-CDR1, VL-CDR2, and VL-CDR3 amino acid sequences selected from the group consisting of: SEQ ID NOs: 23, 24, and 25; SEQ ID NOs: 29, 30, and 31; SEQ ID NOs: 35, 36, and 37; SEQ ID NOs: 46, 47 and 48; and SEQ ID NOs 49, 50 and 51, except for one, two, three, or four amino acid substitutions in at least one of the VL-CDRs. In further embodiments, the VL of the anti-Abeta antibody or antigen-binding fragment thereof comprises VL-CDR1, VL-CDR2, and VL-CDR3 amino acid sequences selected from the group consisting of: SEQ ID NOs: 23, 24, and 25; SEQ ID NOs: 29, 30, and 31; SEQ ID NOs: 35, 36, and 37; SEQ ID NOs: 46, 47 and 48; and SEQ ID NOs 49, 50 and 51.

In certain embodiments, the methods described herein provide an antibody where the heavy chain variable region (VH) and light chain variable region (VL) are from a monoclonal antibody selected from the group consisting of NI-101.10, NI-101.11, NI-101.12, NI-101.13, NI-101.12F6A, NI-101.13A, and NI-101.13B.

In various embodiments, the methods described herein provide an anti-Abeta antibody or antigen-binding fragment thereof that comprises a heavy chain constant region or fragment thereof. In some embodiments, the heavy chain constant region or fragment thereof is human IgG1. In other embodiments, the heavy chain constant region or fragment thereof is mouse IgG2A.

In various embodiments, the methods described herein provide an anti-Abeta antibody or antigen-binding fragment thereof that further comprises a heterologous polypeptide fused thereto.

In various embodiments, the methods described herein provide an anti-Abeta antibody or antigen-binding fragment thereof that is conjugated to an agent selected from the group consisting of cytotoxic agent, a therapeutic agent, cytostatic agent, a biological toxin, a prodrug, a peptide, a protein, an enzyme, a virus, a lipid, a biological response modifier, pharmaceutical agent, a lymphokine, a heterologous antibody or fragment thereof, a detectable label, polyethylene glycol (PEG), and a combination of two or more of any the agents. In some embodiments the cytotoxic agent is selected from the group consisting of a radionuclide, a biotoxin, an enzymatically active toxin, a cytostatic or cytotoxic therapeutic agent, a prodrugs, an immunologically active ligand, a biological response modifier, or a combination of two or more of any the cytotoxic agents. In some embodiments the detectable label is selected from the group consisting of an enzyme, a fluorescent label, a chemiluminescent label, a bioluminescent label, a radioactive label, or a combination of two or more of any the detectable labels.

In other embodiments, the method provides a method of treating an abnormal amyloid condition, where the abnormal amyloid condition is associated with a neurological disease, disorder, injury, or condition. In still further embodiments, the neurological disease, disorder, injury, or condition is in the brain.

In other embodiments, the description herein provides methods of administering an effective amount of an Abeta binding molecule to a subject where the subject has an accumulation of Abeta. In further embodiments, the accumulation of Abeta is associated with a neurological disease, disorder, injury, or condition. In still further embodiments, the neurological disease, disorder, injury, or condition is in the brain.

In some embodiments, the methods provide an Abeta binding molecule that is capable of crossing the blood brain barrier.

In some embodiments, the description herein provides methods of administering an Abeta binding molecule to a subject with a disease, disorder, injury, or condition selected from the group consisting of Alzheimer's disease, Down's Syndrome, head trauma, dementia pugilistica, chronic traumatic encephalopathy (CTE), chronic boxer's encephalopathy, traumatic boxer's encephalopathy, boxer's dementia, punch-drunk syndrome, amyloid deposition associated with aging, mild cognitive impairment, cerebral amyloid angiopathy, Lewy body dementia, vascular dementia, mixed dementia, multi-facet dementia, hereditary cerebral hemorrhage with amyloidosis Dutch type and Icelandic type, glaucoma, Parkinson's disease, Huntington's disease, Creutzfeldt-Jakob disease, cystic fibrosis, or Gaucher's disease and inclusion body myositis. In one embodiment, the disease, disorder, injury, or condition is Alzheimer's disease. In one embodiment, the disease, disorder, injury, or condition is head trauma.

In some embodiments, the description herein provides methods of administering an Abeta binding molecule to a subject where the subject is a mammal. In one embodiment, the mammal is a human.

In some embodiments, the description herein provides methods of administering an Abeta binding molecule to a subject where the Abeta binding molecule is administered intravenously, intramuscularly, subcutaneously, intraperitoneally, intranasally, parenterally or as an aerosol.

### BRIEF DESCRIPTION OF THE DRAWINGS

FIG. 1 shows the progression of AD-like pathology in APP/PS1 mice. The number of Iba1+ microglia was assessed in wild-type (“Non-tg”) and APP/PS1 transgenic mice at 3-4 months of age (A), 11-12 months of age (C) and 17-18 months of age (E) as described in Example 1. Bar height indicates the average number of Iba1+ microglia in the subgranular zone/granular cell layer (SGZ/GCL) and hilus layers at each stage. Short-term memory of wild-type and APP/PS1 transgenic was also assessed using the Y-maze at 3-4 months of age (B), 11-12 months of age (D) and 17-18 months of age (F) as described in Example 1. Bar height indicates the percent alterations. Statistics were calculated using the unpaired t test. Error bars represent SEM. Double asterisks indicates p<0.01, and single asterisk indicates p<0.05.

FIG. 2 shows neurogenesis in APP/PS1 mice. Levels of pH-3, BrdU, PSA-NCAM and BrdU/NeuN were assessed in wild-type (“Non-tg”) and APP/PS1 transgenic mice as described in Example 2 (A). Bar height indicates the percentage difference as compared to controls, and error bars represent the propagation of error. The length of PSA-NCAM+ dendrites and the number of PSA-NCAM+ dendrites per cell body were assessed in wild-type and APP/PS1 transgenic mice as described in Example 2 (B). Statistics were calculated using the unpaired t test. Error bars represent SEM. Double asterisks indicates p<0.01, and single asterisk indicates p<0.05.

FIG. 3 shows the levels of Abeta (A), ThioS (B) and CAA (C) in APP/PS1 transgenic mice treated with a control antibody (“ct ab”) and in APP/PS1 transgenic mice treated with an anti-Abeta antibody (“anti-Abeta”) as described in Example 3. For this analysis, images from sequential sections were processed using ImageJ software to evaluate regions of positive staining over a defined and constant area. Statistics were calculated using the unpaired t test. Error bars represent SEM. Asterisk indicates p<0.05.

FIG. 4 shows the effects of antibodies against-Abeta on neurogenesis in APP/PS1 mice. Numbers of BrdU+ and pH-3+ cells were quantitated in the subgranular zone/granular cell layer (SGZ/GCL) of APP/PS1 transgenic mice treated with a control antibody (“ct ab”) or an anti-Abeta antibody (“anti-Abeta”) as described in Example 4 (A). Immature neurons (identified as PSA-NCAM+ cells) (B) and mature neurons (identified as BrdU+/NeuN+ cells) (C) were quantitated in APP/PS1 transgenic mice treated with a control antibody or an anti-Abeta antibody as described in Example 4. Statistics were calculated using the unpaired t test. Error bars represent SEM. Asterisk indicates p<0.05.

FIG. 5 shows the effects of antibodies against-Abeta on the dendritic arborization of new granular neurons. The number of PSA-NCAM+ dendrites per cell body (A) and length of PSA-NCAM+ dendrites (B) in the subgranular zone were measured in APP/PS1 transgenic mice treated with a control antibody (“ct ab”) or an anti-Abeta antibody (“anti-Abeta”) as described in Example 5. Forty cells were analyzed per group. Levels of synaptophysin (“SYN”) staining in the hippocampal Outer Molecular Layer (“OML”) were measured in transgenic mice treated with a control antibody or an anti-Abeta antibody as described in Example 5 (C). Bar height represents the average staining intensity (A-C). The number of synaptophysin-positive presynaptic terminals were counted in the molecular layer (D). Bar height indicates the number of synaptophysin-positive boutons (D). Statistics were calculated using the unpaired t test. Error bars represent SEM. Double asterisks indicates p<0.01, and single asterisk indicates p<0.05.

FIG. 6 shows the effects of antibodies against-Abeta on angiogenesis. A stereological estimation of the number blood vessels (indicated by lectin staining) in APP/PS1 transgenic mice treated with a control antibody (“ct ab”) or an anti-Abeta antibody (“anti-Abeta”) was performed as described in Example 6. Bar height indicates the number of blood vessels. Statistics were calculated using the unpaired t test. Error bars represent SEM. Double asterisks indicates p<0.01.

FIG. 7 shows the effects of Abeta immunotherapy on dendritic branching of mature retrovirally labeled newly born neurons. Scholl analysis of new mature granule cells labeled with retrovirus expressing GFP was performed. The graph represents the mean number of intersections between dendrites and concentric radii, centered at the cell body, as a function of distance from the soma (n=15 cells per group). Error bars represent S.E.M. Asterisk (*) indicates p<0.05 in non-transgenic vs. APP/PS1, and APP/PS1 vs. APP/PS1 treated with Abeta immunotherapy.

FIG. 8 shows the effects of Abeta immunotherapy on the spine densities of mature retrovirally labeled newly born neurons. High magnification segments from dendrites of new mature neurons were obtained from non-transgenic mice, vehicle-treated APP/PS1 mice, and Abeta-immunotherapy treated APP/PS1 mice (A). The scale bar represents 10 gm. Computer-assisted classification of spines was performed along 40 μm segments to determine the number of mushroom, long-thin, and stubby spines (B-D). The number of mushroom spines in APP/PS1 mice was significantly reduced as compared to non-transgenic mice and was significantly restored by Abeta immunotherapy (B). The number of long-thin spines in APP/PS1 mice was significantly reduced as compared to non-transgenic mice and was significantly restored by Abeta immunotherapy (C). The number of stubby spines in APP/PS1 mice was significantly reduced as compared to non-transgenic mice, and Abeta immunotherapy showed a trend towards rescue (p=0.07) (D). N=50 segments per group. Error bars represent S.E.M. Asterisk (*) indicates p<0.05. Double asterisks (**) indicate p<0.01, and triple asterisks (***) indicate p<0.001. ANOVA followed Bonferroni post hoc tests.

### DETAILED DESCRIPTION OF THE INVENTION

I. Definitions

It is to be noted that the term “a” or “an” entity refers to one or more of that entity; for example, “an antibody,” is understood to represent one or more antibodies. As such, the terms “a” (or “an”), “one or more,” and “at least one” can be used interchangeably herein.

As used herein, the term “polypeptide” is intended to encompass a singular “polypeptide” as well as plural “polypeptides,” and refers to a molecule composed of monomers (amino acids) linearly linked by amide bonds (also known as peptide bonds). The term “polypeptide” refers to any chain or chains of two or more amino acids, and does not refer to a specific length of the product. Thus, peptides, dipeptides, tripeptides, oligopeptides, “protein,” “amino acid chain,” or any other term used to refer to a chain or chains of two or more amino acids, are included within the definition of “polypeptide,” and the term “polypeptide” can be used instead of, or interchangeably with any of these terms.

The term “polypeptide” is also intended to refer to the products of post-expression modifications of the polypeptide, including without limitation glycosylation, acetylation, phosphorylation, amidation, derivatization by known protecting/blocking groups, proteolytic cleavage, or modification by non-naturally occurring amino acids. A polypeptide can be derived from a natural biological source or produced by recombinant technology, but is not necessarily translated from a designated nucleic acid sequence. It can be generated in any manner, including by chemical synthesis.

A polypeptide as described herein can be of a size of about 3 or more, 5 or more, 10 or more, 20 or more, 25 or more, 50 or more, 75 or more, 100 or more, 200 or more, 500 or more, 1,000 or more, or 2,000 or more amino acids. Polypeptides can have a defined three-dimensional structure, although they do not necessarily have such structure. Polypeptides with a defined three-dimensional structure are referred to as folded, and polypeptides that do not possess a defined three-dimensional structure, but rather can adopt a large number of different conformations, and are referred to as unfolded. As used herein, the term glycoprotein refers to a protein coupled to at least one carbohydrate moiety that is attached to the protein via an oxygen-containing or a nitrogen-containing side chain of an amino acid residue, e.g., a serine residue or an asparagine residue.

By an “isolated” polypeptide or a fragment, variant, or derivative thereof is intended a polypeptide that is not in its natural milieu. No particular level of purification is required. For example, an isolated polypeptide can be removed from its native or natural environment. Recombinantly produced polypeptides and proteins expressed in host cells are considered isolated, as are native or recombinant polypeptides that have been separated, fractionated, or partially or substantially purified by any suitable technique.

Also included as polypeptides described herein are fragments, derivatives, analogs, or variants of the foregoing polypeptides, and any combination thereof. The terms “fragment,” “variant,” “derivative” and “analog” when referring to Abeta binding molecules include any polypeptides that retain at least some of the antigen-binding properties of the corresponding native Abeta binding molecule. Fragments of polypeptides include proteolytic fragments, as well as deletion fragments, in addition to specific antibody fragments discussed elsewhere herein. Variants of antibodies and antibody polypeptides include fragments as described above, and also polypeptides with altered amino acid sequences due to amino acid substitutions, deletions, or insertions. Variants can occur naturally or be non-naturally occurring. Non-naturally occurring variants can be produced using art-known mutagenesis techniques. Variant polypeptides can comprise conservative or non-conservative amino acid substitutions, deletions or additions. Derivatives of Abeta binding molecules, e.g., antibodies and antibody polypeptides, are polypeptides that have been altered so as to exhibit additional features not found on the native polypeptide. Examples include fusion proteins. Variant polypeptides can also be referred to herein as “polypeptide analogs.” As used herein a “derivative” of an Abeta binding molecule or fragment thereof, an antibody, or an antibody polypeptide refers to a subject polypeptide having one or more residues chemically derivatized by reaction of a functional side group. Also included as “derivatives” are those peptides that contain one or more naturally occurring amino acid derivatives of the twenty standard amino acids. For example, 4-hydroxyproline can be substituted for proline; 5-hydroxylysine can be substituted for lysine; 3-methylhistidine can be substituted for histidine; homoserine can be substituted for serine; and ornithine can be substituted for lysine.

The term “polynucleotide” is intended to encompass a singular nucleic acid as well as plural nucleic acids, and refers to an isolated nucleic acid molecule or construct, e.g., messenger RNA (mRNA) or plasmid DNA (pDNA). A polynucleotide can comprise a conventional phosphodiester bond or a non-conventional bond (e.g., an amide bond, such as found in peptide nucleic acids (PNA)). The term “nucleic acid” refers to any one or more nucleic acid segments, e.g., DNA or RNA fragments, present in a polynucleotide. By “isolated” nucleic acid or polynucleotide is intended a nucleic acid molecule, DNA or RNA, that has been removed from its native environment. For example, a recombinant polynucleotide encoding an antibody contained in a vector is considered isolated. Further examples of an isolated polynucleotide include recombinant polynucleotides maintained in heterologous host cells or purified (partially or substantially) polynucleotides in solution. Isolated RNA molecules include in vivo or in vitro RNA transcripts of polynucleotides. Isolated polynucleotides or nucleic acids further include such molecules produced synthetically. In addition, polynucleotide or a nucleic acid can be or can include a regulatory element such as a promoter, ribosome binding site, or a transcription terminator.

As used herein, a “coding region” is a portion of nucleic acid that consists of codons that can be translated into amino acids constituting a peptide. Although a “stop codon” (TAG, TGA, or TAA) is not translated into an amino acid, it can be considered to be part of a coding region, but any flanking sequences, for example promoters, ribosome binding sites, transcriptional terminators, introns, and the like, are not part of a coding region. Two or more coding regions can be present in a single polynucleotide construct, e.g., on a single vector, or in separate polynucleotide constructs, e.g., on separate (different) vectors. Furthermore, any vector can contain a single coding region, or can comprise two or more coding regions, e.g., a single vector can separately encode an immunoglobulin heavy chain variable region and an immunoglobulin light chain variable region. In addition, a vector, polynucleotide, or nucleic acid can encode heterologous coding regions, either fused or unfused to a nucleic acid encoding an Abeta binding molecule, an antibody, or fragment, variant, or derivative thereof. Heterologous coding regions include without limitation specialized elements or motifs, such as a secretory signal peptide or a heterologous functional domain.

In certain embodiments, the polynucleotide or nucleic acid is DNA. In the case of DNA, a polynucleotide comprising a nucleic acid that encodes a polypeptide can include a promoter and/or other transcription or translation control elements operably associated with one or more coding regions. An operable association is when a coding region for a gene product, e.g., a polypeptide, is associated with one or more regulatory sequences in such a way as to place expression of the gene product under the influence or control of the regulatory sequence(s). Two DNA fragments (such as a polypeptide coding region and a promoter associated therewith) are “operably associated” if induction of promoter function results in the transcription of mRNA encoding the desired gene product and if the nature of the linkage between the two DNA fragments does not interfere with the ability of the expression regulatory sequences to direct the expression of the gene product or interfere with the ability of the DNA template to be transcribed. Thus, a promoter region would be operably associated with a nucleic acid encoding a polypeptide if the promoter was capable of effecting transcription of that nucleic acid. The promoter can be a cell-specific promoter that directs substantial transcription of the DNA only in predetermined cells. Other transcription control elements, besides a promoter, for example enhancers, operators, repressors, and transcription termination signals, can be operably associated with the polynucleotide to direct cell-specific transcription. Suitable promoters and other transcription control regions are disclosed herein.

A variety of transcription control regions are known to one of skill in the art. These include, without limitation, transcription control regions that function in vertebrate cells, such as, but not limited to, promoter and enhancer segments from cytomegaloviruses (the immediate early promoter, in conjunction with intron-A), simian virus 40 (the early promoter), and retroviruses (such as Rous sarcoma virus). Other transcription control regions include those derived from vertebrate genes such as actin, heat shock protein, bovine growth hormone and rabbit β-globin, as well as other sequences capable of controlling gene expression in eukaryotic cells. Additional suitable transcription control regions include tissue-specific promoters and enhancers as well as lymphokine-inducible promoters (e.g., promoters inducible by interferons or interleukins).

Similarly, a variety of translation control elements are known to one of skill in the art. These include, but are not limited to ribosome binding sites, translation initiation and termination codons, and internal ribosome entry sites (IRES).

In other embodiments, a polynucleotide is RNA, for example, in the form of messenger RNA (mRNA).

Polynucleotide and nucleic acid coding regions can be associated with additional coding regions that encode secretory or signal peptides, which direct the secretion of a polypeptide encoded by a polynucleotide. According to the signal hypothesis, proteins secreted by mammalian cells have a signal peptide or secretory leader sequence that is cleaved from the mature protein once export of the growing protein chain across the rough endoplasmic reticulum has been initiated. One of skill in the art is aware that polypeptides secreted by vertebrate cells generally have a signal peptide fused to the N-terminus of the polypeptide, which is cleaved from the complete or “full length” polypeptide to produce a secreted or “mature” form of the polypeptide. In certain embodiments, the native signal peptide, e.g., an immunoglobulin heavy chain or light chain signal peptide is used, or a functional derivative of that sequence that retains the ability to direct the secretion of the polypeptide that is operably associated with it. Alternatively, a heterologous mammalian signal peptide, or a functional derivative thereof, can be used. For example, the wild-type leader sequence can be substituted with the leader sequence of human tissue plasminogen activator (TPA) or mouse β-glucuronidase.

Unless stated otherwise, the terms “disorder” and “disease” are used interchangeably herein.

An “Abeta binding molecule” as used herein relates to antibodies, and fragments thereof, and can include other non-antibody molecules that bind to Abeta, including but not limited to, antibody mimetics, portions of antibodies that mimic the structure and/or function of an antibody (Qiu et al., Nature Biotechnology 25:921-929 (2007)), hormones, receptors, ligands, major histocompatibility complex (MHC) molecules, chaperones such as heat shock proteins (HSPs) as well as cell-cell adhesion molecules such as members of the cadherin, integrin, C-type lectin and immunoglobulin (Ig) superfamilies. For the sake of clarity only and without restricting the scope of the methods described herein, most of the embodiments described herein are discussed with respect to antibodies and antibody-like molecules that represent the one example of an Abeta binding molecule for the development of therapeutic and diagnostic agents.

Unless specified otherwise, an Abeta binding molecule can bind forms of Abeta including but not limited to Abeta₁₋₄₂ peptide, Abeta₁₋₄₀ peptide, and Abeta₁₋₄₃ peptide, N-terminally truncated Abeta species, C-terminally truncated Abeta species, pyroglutamate-modified Abeta species, e.g., pyroglutamate Abeta3-42, redox-modified Abeta species, Abeta aggregates, dimeric Abeta species, oligomeric Abeta species, fibrillar Abeta, beta-amyloid fibrils, diffuse beta-amyloid deposits, neoepitopes of Abeta generated by protein modification, aggregation or truncation or complex formation of the Abeta peptide and beta-amyloid plaques.

The term “neoepitope” denotes an epitope that is unique for a disease pattern and contained in or formed by a disorder-associated protein that is a pathological variant from an otherwise non-pathological protein and/or deviating from the physiology of the healthy state. Such pathophysiological variants can be formed by means of pathologically altered transcription, pathologically altered translation, post-translational modification, pathologically altered proteolytic processing, pathologically altered complex formation with physiological or pathophysiological interaction partners or cellular structures in the sense of an altered co-localization, or pathologically altered structural conformation—for example aggregation, oligomerization or fibrillation—whose three- or four-dimensional structure differs from the structure of the physiologically active molecule. Moreover, a pathophysiological variant can also be characterized in that it is not located in its usual physiological environment or subcellular compartment. As an example, neoepitopes can be located in the pathologically conspicuous structures in the areas of brain tissues that obviously experience or have already experienced functional damage. Whether a given structure, for example cell or tissue, or protein displays a neoepitope can be verified by reversing the method described below for isolating and characterizing a disorder-associated protein specific Abeta binding molecule in that an Abeta binding molecule, for example antibody identified by said method is used to screen a sample for binding to the antibody, thereby determining the presence of a neoepitope.

The terms “antibody” and “immunoglobulin” are used interchangeably herein. The term “antibody” as used herein is also intended to encompass antibodies, digestion fragments, specified portions and variants thereof, including antibody mimetics or comprising portions of antibodies that mimic the structure and/or function of an antibody or specified fragment or portion thereof, including single chain antibodies and fragments thereof; each containing at least one CDR. See Qiu et al., Nature Biotechnology 25:921-929 (2007). Functional fragments include antigen binding fragments that bind to an Abeta. For example, antibody fragments capable of binding to an Abeta or a portion thereof, including, but not limited to Fab (e.g., by papain digestion), facb (e.g., by plasmin digestion), pFc′ (e.g., by pepsin or plasmin digestion), Fd (e.g., by pepsin digestion, partial reduction and reaggregation), Fv or scFv (e.g., by molecular biology techniques) fragments, are encompassed. Antibody fragments are also intended to include, e.g., domain deleted antibodies, diabodies, linear antibodies, single-chain antibody molecules, and multispecific antibodies formed from antibody fragments.

As will be discussed in more detail below, the term “immunoglobulin” comprises various broad classes of polypeptides that can be distinguished biochemically. Modified versions of each of these classes and isotypes are readily discernable to one of skill in the art in view of the instant disclosure. All immunoglobulin classes are clearly within the scope of the methods described herein, but the following discussion is generally directed to the IgG class of immunoglobulin molecules.

Any antibody or immunoglobulin fragment that contains sufficient structure to specifically bind to an antigen is denoted herein interchangeably as an “antigen binding fragment” or an “immunospecific fragment.”

In the case where there are two or more definitions of a term that is used and/or accepted within the art, the definition of the term as used herein is intended to include all such meanings unless explicitly stated to the contrary. A specific example is the use of the term “complementarity determining region” (“CDR”) to describe the non-contiguous antigen combining sites found within the variable region of both heavy and light chain polypeptides. This particular region has been described by Kabat et al., U.S. Dept. of Health and Human Services, “Sequences of Proteins of Immunological Interest” (1983) and by Chothia et al., J. Mol. Biol. 196:901-917 (1987), which are incorporated herein by reference, where the definitions include overlapping or subsets of amino acid residues when compared against each other. Nevertheless, application of either definition to refer to a CDR of an antibody or variants thereof is intended to be within the scope of the term as defined and used herein. The appropriate amino acid residues that encompass the CDRs as defined by each of the above cited references are set forth below in Table I as a comparison. The exact residue numbers that encompass a particular CDR will vary depending on the sequence and size of the CDR. One of skill in the art can routinely determine the residues comprise a particular CDR given the variable region amino acid sequence of the antibody.

TABLE 1
CDR Definitions¹

| | Kabat | Chothia |
|---|---|---|
| VH CDR1 | 31-35 | 26-32 |
| VH CDR2 | 50-65 | 52-58 |
| VH CDR3 | 95-102 | 95-102 |
| VL CDR1 | 24-34 | 26-32 |
| VL CDR2 | 50-56 | 50-52 |
| VL CDR3 | 89-97 | 91-96 |

¹Numbering of all CDR definitions in Table 1 is according to the numbering conventions set forth by Kabat et al. (see below).

Kabat et al. also defined a numbering system for variable domain sequences that is applicable to any antibody. One of skill in the art can unambiguously assign this system of “Kabat numbering” to any variable domain sequence, without reliance on any experimental data beyond the sequence itself. As used herein, “Kabat numbering” refers to the numbering system set forth by Kabat et al., U.S. Dept. of Health and Human Services, “Sequence of Proteins of Immunological Interest” (1983). Unless otherwise specified, references to the numbering of specific amino acid residue positions in an antibody or antigen-binding fragment, variant, or derivative thereof are according to the Kabat numbering system.

Antibodies or antigen-binding fragments, immunospecific fragments, variants, or derivatives thereof include, but are not limited to, polyclonal, monoclonal, multispecific, human, humanized, primatized, or chimeric antibodies, single chain antibodies, epitope-binding fragments, e.g., Fab, Fab′ and F(ab)₂, Fd, Fvs, single-chain Fvs (scFv), single-chain antibodies, disulfide-linked Fvs (sdFv), fragments comprising either a VL or VH domain, fragments produced by a Fab expression library, and anti-idiotypic (anti-Id) antibodies (including, e.g., anti-Id antibodies to antibodies disclosed herein). ScFv molecules are known in the art and are described, e.g., in U.S. Pat. No. 5,892,019. Immunoglobulin or antibody molecules can be of any type (e.g., IgG, IgE, IgM, IgD, IgA, and IgY), class (e.g., IgG1, IgG2, IgG2a, IgG3, IgG4, IgA1 and IgA2) or subclass of immunoglobulin molecule.

In one embodiment, the antibody is not an IgM or a derivative thereof with a pentavalent structure. Particular, in specific applications, especially therapeutic use, IgMs are less useful than IgGs and other bivalent antibodies or corresponding Abeta binding molecules since IgMs due to their pentavalent structure and lack of affinity maturation often show unspecific cross-reactivities and very low affinity.

Antibody fragments, including single-chain antibodies, can comprise the variable region(s) alone or in combination with the entirety or a portion of the following: hinge region, CH1, CH2, and CH3 domains. Also included are antigen-binding fragments also comprising any combination of variable region(s) with a hinge region, CH1, CH2, and CH3 domains. Antibodies or immunospecific fragments thereof can be from any animal origin including birds and mammals. The antibodies can be human, murine, donkey, rabbit, goat, guinea pig, camel, llama, horse, or chicken antibodies.

In another embodiment, the variable region can be condricthoid in origin (e.g., from sharks). As used herein, “human” antibodies include antibodies having the amino acid sequence of a human immunoglobulin and include antibodies isolated from human patients, human immunoglobulin libraries or from animals transgenic for one or more human immunoglobulins and that do not express endogenous immunoglobulins, as described infra and, for example in, U.S. Pat. No. 5,939,598 by Kucherlapati et al. A human antibody is still “human” even if amino acid substitutions are made in the antibody, e.g., to improve binding characteristics.

As used herein, the term “heavy chain portion” includes amino acid sequences derived from an immunoglobulin heavy chain. A polypeptide comprising a heavy chain portion comprises at least one of: a CH1 domain, a hinge (e.g., upper, middle, and/or lower hinge region) domain, a CH2 domain, a CH3 domain, or a variant or fragment thereof. For example, a binding polypeptide can comprise a polypeptide chain comprising a CH1 domain; a polypeptide chain comprising a CH1 domain, at least a portion of a hinge domain, and a CH2 domain; a polypeptide chain comprising a CH1 domain and a CH3 domain; a polypeptide chain comprising a CH1 domain, at least a portion of a hinge domain, and a CH3 domain, or a polypeptide chain comprising a CH1 domain, at least a portion of a hinge domain, a CH2 domain, and a CH3 domain. In another embodiment, a polypeptide comprises a polypeptide chain comprising a CH3 domain. Further, a binding polypeptide can lack at least a portion of a CH2 domain (e.g., all or part of a CH2 domain). As set forth above, it will be understood by one of skill in the art that these domains (e.g., the heavy chain portions) can be modified such that they vary in amino acid sequence from the naturally occurring immunoglobulin molecule.

In certain antibodies, or antigen-binding fragments, variants, or derivatives thereof disclosed herein, the heavy chain portions of one polypeptide chain of a multimer are identical to those on a second polypeptide chain of the multimer. Alternatively, heavy chain portion-containing monomers are not identical. For example, each monomer can comprise a different target binding site, forming, for example, a bispecific antibody.

The heavy chain portions of a binding polypeptide for use in the diagnostic and treatment methods disclosed herein can be derived from different immunoglobulin molecules. For example, a heavy chain portion of a polypeptide can comprise a CH1 domain derived from an IgG1 molecule and a hinge region derived from an IgG3 molecule. In another example, a heavy chain portion can comprise a hinge region derived, in part, from an IgG1 molecule and, in part, from an IgG3 molecule. In another example, a heavy chain portion can comprise a chimeric hinge derived, in part, from an IgG1 molecule and, in part, from an IgG4 molecule.

As used herein, the term “light chain portion” includes amino acid sequences derived from an immunoglobulin light chain. The light chain portion can comprise at least one of a VL or CL domain.

The minimum size of a peptide or polypeptide epitope for an antibody is thought to be about four to five amino acids. Peptide or polypeptide epitopes can contain at least seven, at least nine or between at least about 15 to about 30 amino acids. Since a CDR can recognize an antigenic peptide or polypeptide in its tertiary form, the amino acids comprising an epitope need not be contiguous, and in some cases, are not even on the same peptide chain. A peptide or polypeptide epitope recognized by antibodies can contain a sequence of at least 4, at least 5, at least 6, at least 7, at least 8, at least 9, at least 10, at least 15, at least 20, at least 25, or between about 15 to about 30 contiguous or non-contiguous amino acids of Abeta.

By “specifically binds,” or “specifically recognizes,” used interchangeably herein, it is generally meant that an Abeta binding molecule, e.g., an antibody, binds to Abeta via its antigen binding domain, and that the binding entails some complementarity between the antigen binding domain and Abeta. As used herein terms such as “absence of cross-reactivity”, “specific,” “specifically recognizing,” “specifically binding,” and the like refer to the Abeta binding molecule's ability to discriminate between Abeta and another epitope. According to this definition, an Abeta binding molecule is said to “specifically bind” to Abeta when it binds to Abeta more readily than it would bind to a random, unrelated epitope. The term “specificity” is used herein to qualify the relative affinity by which a certain Abeta binding molecule binds to Abeta compared to another epitope. For example, Abeta binding molecule “A” can be deemed to have a higher specificity for Abeta than Abeta binding molecule “B,” or Abeta binding molecule “A” can be said to bind to Abeta with a higher specificity than it has for another epitope. Thus, the Abeta binding molecule can have a preferential binding affinity to Abeta over another epitope by a factor of at least two, at least 5, more than by a factor of 10, more than by a factor of 50 and or more than by a factor of 100. Furthermore, the relative KD of the Abeta binding molecule, e.g., antibody for the Abeta can be at least 10-fold less, at least 100-fold less or more than the KD for binding that antibody to other ligands or to the native counterpart of the disease-associated protein.

The phrases “disease-associated protein specific” and “neoepitope specific” are used interchangeably herein with the term “specifically recognizing a neoepitope”. These terms refer to the Abeta binding molecule's ability to discriminate between the neoepitope of a disorder-associated protein and the native protein in its wild type form and natural context. Thus, the Abeta binding molecule can have a preferential binding affinity to the neoepitope over the native protein antigen by a factor of at least two, at least 5, more than by a factor of 10, more than by a factor of 50 or more than by a factor of 100. Furthermore, the relative KD of the Abeta binding molecule, e.g., antibody for the specific target epitope, e.g. neoepitope can be at least 10-fold less, at least 100-fold less or more than the KD for binding that antibody to other ligands or to the native counterpart of the disease-associated protein.

By “preferentially binds,” it is meant that the Abeta binding molecule, e.g., antibody, specifically binds to a epitope more readily than it would bind to a related, similar, homologous, or analogous epitope. Thus, an antibody that “preferentially binds” to a given epitope would more likely bind to that epitope than to a related epitope, even though such an antibody can cross-react with the related epitope.

By way of non-limiting example, an Abeta binding molecule, e.g., an antibody can be considered to bind a first epitope preferentially if it binds said first epitope with a dissociation constant (KD) that is less than the antibody's KD for the second epitope. In another non-limiting example, an antibody can be considered to bind a first antigen preferentially if it binds the first epitope with an affinity that is at least one order of magnitude less than the antibody's KD for the second epitope. In another non-limiting example, an antibody can be considered to bind a first epitope preferentially if it binds the first epitope with an affinity that is at least two orders of magnitude less than the antibody's KD for the second epitope.

In another non-limiting example, an Abeta binding molecule, e.g., an antibody can be considered to bind a first epitope preferentially if it binds the first epitope with an off rate (k(off)) that is less than the antibody's k(off) for the second epitope. In another non-limiting example, an antibody can be considered to bind a first epitope preferentially if it binds the first epitope with an affinity that is at least one order of magnitude less than the antibody's k(off) for the second epitope. In another non-limiting example, an antibody can be considered to bind a first epitope preferentially if it binds the first epitope with an affinity that is at least two orders of magnitude less than the antibody's k(off) for the second epitope.

An Abeta binding molecule, e.g., an antibody or antigen-binding fragment, variant, or derivative disclosed herein can be said to bind a target disclosed herein or a fragment or variant thereof with an off rate (k(off)) of less than or equal to 5×10⁻² sec⁻¹, 10⁻² sec⁻¹, 5×10⁻³ sec⁻¹ or 10⁻³ sec . In some embodiments, an antibody can be said to bind a target disclosed herein or a fragment or variant thereof with an off rate (k(off)) less than or equal to 5×10^{÷1} sec⁻¹, 10⁻⁴ sec⁻¹, 5×10⁻⁵ sec⁻¹, or 10⁻⁵ sec⁻¹ 5×10⁻⁶ sec⁻¹, 10⁻⁶ sec⁻¹, 5×10⁻⁷ sec⁻¹ or 10⁻⁷ sec⁻¹.

An Abeta binding molecule, e.g., an antibody or antigen-binding fragment, variant, or derivative disclosed herein can be said to bind a target disclosed herein or a fragment or variant thereof with an on rate (k(on)) of greater than or equal to 10³ M⁻¹ sec⁻¹, 5×10³ M⁻¹ sec⁻¹, 10⁴ M⁻¹ sec⁻¹ or 5×10⁴ M⁻¹ sec⁻¹. In some embodiments, an antibody can be said to bind a target disclosed herein or a fragment or variant thereof with an on rate (k(on)) greater than or equal to 10⁵ M⁻¹ sec⁻¹, 5×10⁵ M⁻¹ sec⁻¹, 10⁶ M⁻¹ sec⁻¹, or 5×10⁶ M⁻¹ sec⁻¹ or 10⁷ M⁻¹ sec⁻¹.

An Abeta binding molecule, e.g., an antibody is said to competitively inhibit binding of a reference antibody to a given epitope if it preferentially binds to that epitope to the extent that it blocks, to some degree, binding of the reference antibody to the epitope. Competitive inhibition can be determined by any method known in the art, for example, competition ELISA assays. An antibody can be said to competitively inhibit binding of the reference antibody to a given epitope by at least 90%, at least 80%, at least 70%, at least 60%, or at least 50%.

As used herein, the term “affinity” refers to a measure of the strength of the binding of an individual epitope with the epitope binding site of an Abeta binding molecule, e.g., a CDR of an immunoglobulin molecule. See, e.g., Harlow et al., Antibodies: A Laboratory Manual, (Cold Spring Harbor Laboratory Press, 2nd ed. 1988) at pages 27-28. As used herein, the term “avidity” refers to the overall stability of the complex between a population of antigen Abeta binding molecules and an antigen, that is, the functional combining strength of an immunoglobulin mixture with the antigen. See, e.g., Harlow at pages 29-34. Avidity is related to both the affinity of individual immunoglobulin molecules in the population with specific epitopes, and also the valencies of the immunoglobulins and the antigen. For example, the interaction between a bivalent monoclonal antibody and an antigen with a highly repeating epitope structure, such as a polymer, would be one of high avidity.

Abeta binding molecules, e.g., antibodies or antigen-binding fragments, variants or derivatives thereof can also be described or specified in terms of their cross-reactivity. As used herein, the term “cross-reactivity” refers to the ability of an antibody, specific for one antigen, to react with a second antigen; a measure of relatedness between two different antigenic substances. Thus, an antibody is cross reactive if it binds to an epitope other than the one that induced its formation. The cross reactive epitope generally contains many of the same complementary structural features as the inducing epitope, and in some cases, can actually fit better than the original.

For example, certain antibodies have some degree of cross-reactivity, in that they bind related, but non-identical epitopes, e.g., epitopes with at least 95%, at least 90%, at least 85%, at least 80%, at least 75%, at least 70%, at least 65%, at least 60%, at least 55%, and at least 50% identity (as calculated using methods known in the art and described herein) to a reference epitope. An antibody can be said to have little or no cross-reactivity if it does not bind epitopes with less than 95%, less than 90%, less than 85%, less than 80%, less than 75%, less than 70%, less than 65%, less than 60%, less than 55%, and less than 50% identity (as calculated using methods known in the art and described herein) to a reference epitope. An antibody can be deemed “highly specific” for a certain epitope, if it does not bind any other analog, ortholog, or homolog of that epitope.

Abeta binding molecules, e.g., antibodies or antigen-binding fragments, variants or derivatives thereof can also be described or specified in terms of their binding affinity to a target. The binding affinities include those with a dissociation constant or Kd less than 5×10⁻² M, 10⁻² M, 5×10⁻³ M, 10⁻³ M, 5×10⁻⁴ M, 10⁻⁴ M, 5×10⁻⁵ M, 10⁻⁵ M, 5×10⁻⁶ M, 10⁻⁶ M, 5×10⁻⁷ M, 10⁻⁷ M, 5×10⁻⁸ M, 10⁻⁸ M, 5×10⁻⁹ M, 5×10⁻¹⁰ M, 10⁻¹⁰ M, 5×10⁻¹¹ M, 10⁻¹¹ M, 5×10⁻¹² M, 10⁻¹² M, 5×10⁻¹³ M, 10⁻¹³ M, 5×10⁻¹⁴ M, 10⁻¹⁴ M, 5×10⁻¹⁵ M, or 10⁻¹⁵-M.

As previously indicated, the subunit structures and three dimensional configuration of the constant regions of the various immunoglobulin classes are well known. As used herein, the term “VH domain” includes the amino terminal variable domain of an immunoglobulin heavy chain and the term “CH1 domain” includes the first (most amino terminal) constant region domain of an immunoglobulin heavy chain. The CH1 domain is adjacent to the VH domain and is amino terminal to the hinge region of an immunoglobulin heavy chain molecule.

As used herein the term “CH2 domain” includes the portion of a heavy chain molecule that extends, e.g., from about residue 244 to residue 360 of an intact heavy chain using conventional numbering schemes (residues 244 to 360, Kabat numbering system; and residues 231-340, EU numbering system; see Kabat E A et al. op. cit. The CH2 domain is unique in that it is not closely paired with another domain. Rather, two N-linked branched carbohydrate chains are interposed between the two CH2 domains of an intact native IgG molecule. It is also well documented that the CH3 domain extends from the CH2 domain to the C-terminal of the IgG molecule and comprises approximately 108 residues.

As used herein, the term “hinge region” includes the portion of a heavy chain molecule that joins the CH1 domain to the CH2 domain. This hinge region comprises approximately 25 residues and is flexible, thus allowing the two N-terminal antigen binding regions to move independently. Hinge regions can be subdivided into three distinct domains: upper, middle, and lower hinge domains (Roux et al., J. Immunol. 161:4083 (1998)).

As used herein the term “disulfide bond” includes the covalent bond formed between two sulfur atoms. The amino acid cysteine comprises a thiol group that can form a disulfide bond or bridge with a second thiol group. In most naturally occurring IgG molecules, the CH1 and CL regions are linked by a disulfide bond and the two heavy chains are linked by two disulfide bonds at positions corresponding to 239 and 242 using the Kabat numbering system (position 226 or 229, EU numbering system).

As used herein, the term “engineered antibody” refers to an antibody in which the variable domain in either the heavy and light chain or both is altered by at least partial replacement of one or more CDRs from an antibody of known specificity and, if necessary, by partial framework region replacement and sequence changing. Although the CDRs can be derived from an antibody of the same class or even subclass as the antibody from which the framework regions are derived, it is envisaged that the CDRs will be derived from an antibody of different class or from an antibody from a different species. An engineered antibody in which one or more “donor” CDRs from a non-human antibody of known specificity is grafted into a human heavy or light chain framework region is referred to herein as a “humanized antibody.” It is not always necessary to replace all of the CDRs with the complete CDRs from the donor variable region to transfer the antigen binding capacity of one variable domain to another. Rather, it can only be necessary to transfer those residues that are necessary to maintain the activity of the target binding site.

As discussed herein, in some embodiments, the starting material of the described process is a humanized, or, in some embodiments, a murinized monoclonal antibody. In other words, a suitable selected monoclonal antibody can comprise one or more CDRs from an animal antibody, the antibody having been modified in such a way so as to be less immunogenic in a human or mouse than the parental animal antibody. As is known, animal antibodies can be humanized using a number of methodologies, including chimeric antibody production, CDR grafting (including reshaping), and antibody resurfacing. In general, chimeric antibodies are made by transferring the constant regions from a human antibody onto an antibody from a non-human animal, and CDR grafting involves transferring CDR regions, corresponding to the domains that provide specific binding, from a non-human antibody onto a human antibody framework. Resurfacing involves substituting framework amino acids that are exposed in a non-human antibody (i.e., on the exterior surface of the antibody) with equivalent exposed residues of a human antibody. Suitable methods for humanizing or murinizing antibodies can be found in U.S. Pat. Nos. 6,331,415 B1, 5,225,539, 6,342,587, 4,816,567, 5,639,641, 6,180,370, 5,693,762, 4,816,397, 5,693,761, 5,530,101, 5,585,089, 6,329,551, and, in particular in U.S. Patent Application No. 60/404,117, filed Aug. 15, 2002, which is specifically incorporated herein by reference in its entirety.

As used herein the term “properly folded polypeptide” includes polypeptides in which all of the functional domains comprising the polypeptide are distinctly active. As used herein, the term “improperly folded polypeptide” includes polypeptides in which at least one of the functional domains of the polypeptide is not active. In one embodiment, a properly folded polypeptide comprises polypeptide chains linked by at least one disulfide bond and, conversely, an improperly folded polypeptide comprises polypeptide chains not linked by at least one disulfide bond.

As used herein the term “engineered” includes manipulation of nucleic acid or polypeptide molecules by synthetic means (e.g. by recombinant techniques, in vitro peptide synthesis, by enzymatic or chemical coupling of peptides or some combination of these techniques).

As used herein, the terms “linked,” “fused” or “fusion” are used interchangeably. These terms refer to the joining together of two more elements or components, by whatever means including chemical conjugation or recombinant means. An “in-frame fusion” refers to the joining of two or more polynucleotide open reading frames (ORFs) to form a continuous longer ORF, in a manner that maintains the correct translational reading frame of the original ORFs. Thus, a recombinant fusion protein is a single protein containing two or more segments that correspond to polypeptides encoded by the original ORFs (which segments are not normally so joined in nature.) Although the reading frame is thus made continuous throughout the fused segments, the segments can be physically or spatially separated by, for example, in-frame linker sequence. For example, polynucleotides encoding the CDRs of an immunoglobulin variable region can be fused, in-frame, but be separated by a polynucleotide encoding at least one immunoglobulin framework region or additional CDR regions, as long as the “fused” CDRs are co-translated as part of a continuous polypeptide.

In the context of polypeptides, a “linear sequence” or a “sequence” is an order of amino acids in a polypeptide in an amino to carboxyl terminal direction in which residues that neighbor each other in the sequence are contiguous in the primary structure of the polypeptide.

The term “expression” as used herein refers to a process by which a nucleic acid is used to produce a biochemical, for example, an RNA or polypeptide. The process includes any manifestation of the functional presence of the gene within the cell including, without limitation, gene knockdown as well as both transient expression and stable expression. It includes, without limitation, transcription of the nucleic acid into messenger RNA (mRNA), transfer RNA (tRNA), small hairpin RNA (shRNA), small interfering RNA (siRNA) or any other RNA product, and the translation of such mRNA into polypeptide(s). If the final desired product is a biochemical, expression includes the creation of that biochemical and any precursors. Expression of a gene produces a “gene product.” As used herein, a gene product can be either a nucleic acid, e.g., a messenger RNA produced by transcription of a gene, or a polypeptide that is translated from a transcript. Gene products described herein further include nucleic acids with post-transcriptional modifications, e.g., polyadenylation, or polypeptides with post-translational modifications, e.g., methylation, glycosylation, the addition of lipids, association with other protein subunits, proteolytic cleavage, and the like.

The terms “treatment”, “treating”, “treat” and the like are used herein to generally mean obtaining a desired pharmacological and/or physiological effect. Encompassed by this definition are both therapeutic treatment and prophylactic or preventative measures, wherein the object is to prevent or slow down (lessen) an undesired physiological change or disorder, or ameliorate a disease symptom, such as the development or spread of Alzheimer's disease. The effect can be prophylactic in terms of completely or partially preventing a disease or symptom thereof and/or can be therapeutic in terms of partially or completely curing a disease and/or adverse effect attributed to the disease. The term “treatment” as used herein covers any treatment of a disease in a mammal, particularly a human, and includes: (a) preventing the disease from occurring in a subject that can be predisposed to the disease but has not yet been diagnosed as having it; (b) inhibiting the disease, e.g., arresting or slowing its development; or (c) relieving the disease, e.g., causing regression of the disease. Beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those prone to have the condition or disorder or those in which the manifestation of the condition or disorder is to be prevented.

Methods of treating Alzheimer's disease or related disorders such as dementia pugilistica include methods of treating a subject having a likely diagnosis of such disease. Methods of diagnosing Alzheimer's disease and related disorders are known in the art. Symptoms of Alzheimer's disease are known in the art, and methods of evaluating symptoms are known in the art.

By “subject” or “individual” or “animal” or “patient” or “mammal,” is meant any subject, particularly a mammalian subject, e.g., a human patient, for whom diagnosis, prognosis, prevention, or therapy is desired. The terms also encompass a mammal, e.g., a human, in need of treatment for an injury, condition, disorder or disease.

II. Methods of Using Abeta Binding Molecules

The present description generally relates to methods for using antibodies and other Abeta binding molecules that are capable of recognizing an Abeta. One embodiment described herein provides methods for promoting neurogenesis in a subject, the method comprising administering to a subject an effective amount of an Abeta binding molecule.

The phrase “neurogenesis” as used herein, refers to an increase in neurons. The increase can be, for example, the result of increased proliferation, increased neuronal differentiation, promotion of proper development and integration of immature neurons into functional neuronal networks and/or increased survival of neurons. Neurogenesis can be assessed, for example, by increased mitotic activity of neuronal stem cells, increased number of immature and/or mature neurons, and/or increased integration of immature neurons into functional neural networks. Methods of increasing and assessing neurogenesis are illustrated, by way of example, in the experiments described in the present application.

Additionally, the present description is directed to a method of promoting angiogenesis in a subject, the method comprising administering to a subject an effective amount of an Abeta binding molecule.

The phrase “angiogenesis” as used herein, refers to the growth of blood vessels and can include, for example, an increase in number of blood vessels and/or in the length or size of blood vessels. Methods of increasing and assessing angiogenesis are illustrated, by way of example, in the experiments described in the present application.

An additional embodiment provides methods for promoting synaptic activity in a subject, the method comprising administering to a subject an effective amount of an Abeta binding molecule.

The methods described herein also include a method for promoting the dendritic arborization of granular neurons in a subject, the method comprising administering to a subject an effective amount of an Abeta binding molecule. In the methods described herein, the subject can have an accumulation of beta-amyloid. The accumulation of beta-amyloid can be associated with a neurological disease, disorder, injury or condition. In one embodiment, the neurological disease, disorder, injury or condition is in the brain.

The methods described herein further include a method of treating an abnormal amyloid condition in a subject, the method comprising administering to a subject in need thereof a therapeutically effective amount of an Abeta binding molecule, wherein the Abeta binding molecule promotes neurogenesis. In certain embodiments described herein, the abnormal amyloid condition is associated with a neurological disease, disorder, injury, or condition. In one embodiment, the neurological disease, disorder, injury or condition is in the brain.

In certain embodiments described herein, the Abeta binding molecule is capable of crossing the blood brain barrier. In further embodiments, the Abeta binding molecule can be an anti-Abeta antibody or antigen-binding fragment thereof.

Neurological diseases, disorders, injuries, or conditions that can be treated or ameliorated by the methods described herein include but are not limited to Alzheimer's disease, Down's Syndrome, head trauma, dementia pugilistica, chronic traumatic encephalopathy (CTE), chronic boxer's encephalopathy, traumatic boxer's encephalopathy, boxer's dementia, punch-drunk syndrome, amyloid deposition associated with aging, mild cognitive impairment, cerebral amyloid angiopathy, Lewy body dementia, vascular dementia, mixed dementia, multi-facet dementia, hereditary cerebral hemorrhage with amyloidosis Dutch type and Icelandic type, glaucoma, Parkinson's disease, Huntington's disease, Creutzfeldt-Jakob disease, cystic fibrosis, or Gaucher's disease and inclusion body myositis. In one embodiment, the disease, disorder, injury, or condition is Alzheimer's disease. In another embodiment, the disease, disorder, injury, or condition is head trauma.

In certain embodiments described herein, the subject is a mammal. In one embodiment described herein, the mammal is a human.

III. Antibodies

The Abeta binding molecules for use in the methods described herein include the Abeta binding molecules, e.g., antibodies and binding fragments, variants, and derivatives thereof shown in Table 2 and 3. The methods described herein include the use of an antibody, or antigen-binding fragment, variant or derivatives thereof, where the antibody specifically binds to the same epitope as a reference antibody selected from the group consisting of NI-101.10, NI-101.11, NI-101.12, NI-101.13, NI-101.12F6A, NI-101.13A, and NI-101.13B.

Antibodies for use in the methods described herein also include an antibody, or antigen-binding fragment, variant or derivatives thereof, where the antibody competitively inhibits a reference antibody selected from the group consisting of NI-101.10, NI-101.11, NI-101.12, NI-101.13, NI-101.12F6A, NI-101.13A, and NI-101.13B from binding to Abeta.

Antibodies for use in the methods described herein further include an antibody, or antigen-binding fragment, variant or derivatives thereof, where the antibody comprises an antigen binding domain identical to that of an antibody selected from the group consisting of NI-101.10, NI-101.11, NI-101.12, NI-101.13, NI-101.12F6A, NI-101.13A, and NI-101.13B.

The present description further exemplifies several such Abeta binding molecules, e.g., antibodies and binding fragments thereof, which can be used in the methods described herein, which can be characterized by comprising in their variable region, e.g., binding domain at least one complementarity determining region (CDR) of the V_{H} and/or V_{L} variable region comprising any one of the amino acid sequences depicted in Table 2 (V_{H}) and Table 3 (V_{L}).

TABLE 2
Amino acid sequences of the V_{H} region

| Antibody | Variable heavy chain sequence |
|---|---|
| NI-101.10 | EVQLVQSGGGVVQPGRSLRLSCAASGFAFSSYGIHW |
| | VRQAPGKGLEWVAVIWFDGTKKYYTDSVKGRFTISR |
| | DNSKNTLYLQMNTLRAEDTAVYYCARDRGIGARRGP |
| | YYMDVWGKGTTVTVSS |
| | (SEQ ID NO: 4) |
| NI-101.11 | EVQLVQSGGGVVQPGRSLRLSCAASGFAFSSYGMHW |
| | VRQAPGKGLEWVAVIWFDGTKKYYTDSVKGRFTISR |
| | DNSKNTLYLQMNTLRAEDTAVYYCARDRGIGARRGP |
| | YYMDVWGKGTTVTVSS |
| | (SEQ ID NO: 6) |
| NI-101.12 | EVQLVESGPGLVKPAETLSLTCTVSGGSIRSGSICW |
| | YWIRQPPGKGLEWIGYFCYSGATFYTPSLRGRLTIS |
| | VDASKNQLSLSLSSVTAADTAVYYCARRAGENSGGI |
| | EPYYGMDVWGQGTTVTVSS |
| | (SEQ ID NO: 10) |
| NI-101.13 | QVQLQESGPGLVKPSETLSLTCTVSGGSISRRSYYW |
| | GWIRQSPGKGLEWSGSIHYSGSTYYNPSLKSRVTIS |
| | VDTSKNQFSLKLSSVTAADTAVYYCARSRWGSSWVF |
| | DYWGQGTLVTVSS |
| | (SEQ ID NO: 14) |
| NI-101.12F | QVQLVESGGGVVQPGRSLRLSCAASGFAFSSYGMHW |
| 6A | VRQAPGKGLEWVAVIWFDGTKKYYTDSVKGRFTISR |
| | DNSKNTLYLQMNTLRAEDTAVYYCARDRGIGARRGP |
| | YYMDVWGKGTTVTVSS |
| | (SEQ ID NO: 39) |
| NI-101.13A | QVQLQESGPGLVKPSETLSLTCTVSGGSISRRSYYW |
| | GWIRQSPGKGLEWSGSIHYSGSTYYNPSLKSRVTIS |
| | VDTSKNQFSLKLSSVTAADTAVYYCARSRWGSSWVF |
| | DYWGQGTLVTVSS |
| | (SEQ ID NO: 42) |
| NI-101.13B | QVQLQESGPGLVKPSETLSLTCTVSGGSISRRSYYW |
| | GWIRQSPGKGLEWSGSIHYSGSTYYNPSLKSRVTIS |
| | VDTSKNQFSLKLSSVTAADTAVYYCARSRWGSSWVF |
| | DYWGQGTLVTVSS |
| | (SEQ ID NO: 43) |

TABLE 3
Amino acid sequences of the V_{L} region

| | Variable light chain sequence |
|---|---|
| Antibody | (kappa or lambda) |
| NI-101.10 | EIVLTQSPSSLSASVGDRVTITCRASQSISSYLNWY |
| | QQKPGKAPKLLIYAASSLQSGVPSRFSGSGSGTDFT |
| | LTISSLQPEDFATYYCQQSYSTPLTFGGGTKLEIKR |
| | (SEQ ID NO: 8) |
| NI-101.11 | EIVLTQSPSSLSASVGDRVTITCRASQSISSYLNWY |
| | QQKPGKAPKLLIYAASSLQSGVPSRFSGSGSGTDFT |
| | LTISSLQPEDFATYYCQQSYSTPLTFGGGTKLEIKR |
| | (SEQ ID NO: 8) |
| NI-101.12 | DEIVLTQSPSSLSASIGDRVTITCRASESINKYVNW |
| | YQQKPGKAPKLLIYAASSLQSGAPSRVSGSGFGRDF |
| | SLTISGLQAEDFGAYFCQQSYSAPYTFGQGTKVEIK |
| | RT |
| | (SEQ ID NO: 12) |
| NI-101.13 | QSVLTQPPSASGTPGQRVTISCSGSSSNIGSNYVYW |
| | YQQPPGTAPKLLIYRNNQRPSGVPDRFSGSKSGTSA |
| | SLAISGLRSEDEADYYCAAWDDSLSGYVFGTGTKVT |
| | VLG |
| | (SEQ ID NO: 16) |
| NI-101.12F | DIQMTQSPSSLSASVGDRVTITCRASQSISSYLNWY |
| 6A | QQKPGKAPKLLIYAASSLQSGVPSRFSGSGSGTDFT |
| | LTISSLQPEDFATYYCQQSYSTPLTFGGGTKVEIKR |
| | (SEQ ID NO: 41) |
| NI-101.13A | DIQLTQSPSSLSASVGDRVTITCRASQSISSYLNWY |
| | QQKPGKAPKLLIYAASSLQSGVPSRFSGSGSGTDFT |
| | LTISSLQPEDFATYYCQQSYSTRTFGQGTKVEIKR |
| | (SEQ ID NO: 44) |
| NI-101.13B | DIQLTQSPSTLSASVGDRVTITCRASQSISSWLAWY |
| | QQIPGKAPKWYKASSLESGVPSRFSGSGSGTEFTLT |
| | ISSLQPDDFATYYCQQYNSYSRTFGQGTKLEIKR |
| | (SEQ ID NO: 45) |

The corresponding nucleotide sequences encoding the above-identified variable regions are set forth below. An exemplary set of CDRs of the above amino acid sequences of the V_{H} and/or V_{L} region as depicted in Tables 2 and 3 are given in Table 4. However, one of skill in the art is well aware of the fact that in addition or alternatively CDRs can be used, which differ in their amino acid sequence from those set forth in Table 4 by one, two, three or even more amino acids in case of CDR2 and CDR3.

TABLE 4
Denomination of CDR protein sequences in
Kabat Nomenclature of V_{H} and V_{L} regions

| | Variable | Variable |
|---|---|---|
| Antibody | heavy chain | light chain |
| NI-101.10 | | |
| CDR1 | SYGIH | RASQSISSYLN |
| | (SEQ ID NO: 17) | (SEQ ID NO: 23) |
| CDR2 | VIWFDGTKKYYTDSVKG | AASSLQS |
| | (SEQ ID NO: 18) | (SEQ ID NO: 24) |
| CDR3 | DRGIGARRGPYYMDV | QQSYSTPLT |
| | (SEQ ID NO: 19) | (SEQ ID NO: 25) |
| NI-101.11 | | |
| CDR1 | SYGMH | RASQSISSYLN |
| | (SEQ ID NO: 20) | (SEQ ID NO: 23) |
| CDR2 | VIWFDGTKKYYTDSVKG | AASSLQS |
| | (SEQ ID NO: 21) | (SEQ ID NO: 24) |
| CDR3 | DRGIGARRGPYYMDV | QQSYSTPLT |
| | (SEQ ID NO: 22) | (SEQ ID NO: 25) |
| NI-101.12 | | |
| CDR1 | SGSIC | RASESINKYVN |
| | (SEQ ID NO: 26) | (SEQ ID NO: 29) |
| CDR2 | WIGYFCYSGATFYTPSLRG | AASSLQS |
| | (SEQ ID NO: 27) | (SEQ ID NO: 30) |
| CDR3 | RAGENSGGIEPYYGMDV | QQSYSAPYT |
| | (SEQ ID NO: 28) | (SEQ ID NO: 31) |
| NI-101.13 | | |
| CDR1 | RRSYYWG | SGSSSNIGSNYVY |
| | (SEQ ID NO: 32) | (SEQ ID NO: 35) |
| CDR2 | SIHYSGSTYYNPSLKS | RNNQRPS |
| | (SEQ ID NO: 33) | (SEQ ID NO: 36) |
| CDR3 | SRWGSSWVFDY | AAWDDSLSGYV |
| | (SEQ ID NO: 34) | (SEQ ID NO: 37) |
| NI-101.12F6A | | |
| CDR1 | SYGMH | RASQSISSYLN |
| | (SEQ ID NO: 20) | (SEQ ID NO: 23) |
| CDR2 | VIWFDGTKKYYTDSVKG | AASSLQS |
| | (SEQ ID NO: 21) | (SEQ ID NO: 24) |
| CDR3 | DRGIGARRGPYYMDV | QQSYSTPLT |
| | (SEQ ID NO: 22) | (SEQ ID NO: 25) |
| NI-101.13A | | |
| CDR1 | RRSYYWG | RASQSISSYLN |
| | (SEQ ID NO: 32) | (SEQ ID NO: 46) |
| CDR2 | SIHYSGSTYYNPSLKS | AASSLQS |
| | (SEQ ID NO: 33) | (SEQ ID NO: 47) |
| CDR3 | SRWGSSWVFDY | QQSYSTRT |
| | (SEQ ID NO: 34) | (SEQ ID NO: 48) |
| NI-101.13B | | |
| CDR1 | RRSYYWG | RASQSISSWLA |
| | (SEQ ID NO: 32) | (SEQ ID NO: 49) |
| CDR2 | SIHYSGSTYYNPSLKS | KASSLES |
| | (SEQ ID NO: 33) | (SEQ ID NO: 50) |
| CDR3 | SRWGSSWVFDY | QQYNSYSRT |
| | (SEQ ID NO: 34) | (SEQ ID NO: 51) |

In one embodiment, the antibodies for use in the methods described herein comprise, consist essentially of, or consist of an immunoglobulin heavy chain variable region (VH) at least 80%, 85%, 90%, 95%, or 100% identical to a reference amino acid sequence selected from the group consisting of SEQ ID NO: 4, SEQ ID NO: 6, SEQ ID NO: 10, SEQ ID NO: 14, SEQ ID NO: 39, SEQ ID NO: 42, and SEQ ID NO: 43.

In another embodiment, the antibodies for use in the methods described herein comprise, consist essentially of, or consist of an immunoglobulin light chain variable region (VL) at least 80%, 85%, 90%, 95%, or 100% identical to reference amino acid sequence selected from the group consisting of SEQ ID NO: 8, SEQ ID NO: 12, SEQ ID NO: 16, SEQ ID NO: 41, SEQ ID NO: 44, and SEQ ID NO:45.

In another embodiment, the antibodies for use in the methods described herein comprise, consist essentially of, or consist of an immunoglobulin heavy chain variable region (VH) and an immunoglobulin light chain variable region (VL) at least 80%, 85%, 90%, 95%, or 100% identical to reference amino acid sequences selected from the group consisting of SEQ ID NO: 4 and SEQ ID NO: 8; SEQ ID NO: 6 and SEQ ID NO: 8; SEQ ID NO: 10 and SEQ ID NO: 12; SEQ ID NO: 14 and SEQ ID NO: 16; SEQ ID NO: 39 and SEQ ID NO: 41; SEQ ID NO: 42 and SEQ ID NO: 44; and SEQ ID NO: 43 and SEQ ID NO: 45.

In a still further embodiment, the antibodies for use in the methods described herein comprise, consist essentially of, or consist of an immunoglobulin heavy chain variable region (VH) identical, except for 1, 2, 3, 4, 5, 6, 7, 8, 9, 10, 15, 20, 25, 30, 50 or fewer conservative amino acid substitutions, to a reference amino acid sequence selected from the group consisting of SEQ ID NO: 4, SEQ ID NO: 6, SEQ ID NO: 10, SEQ ID NO: 14, SEQ ID NO: 39, SEQ ID NO: 42, and SEQ ID NO: 43.

In a still further embodiment, the antibodies for use in the methods described herein comprise, consist essentially of, or consist of an immunoglobulin light chain variable region (VL) identical, except for 1, 2, 3, 4, 5, 6, 7, 8, 9, 10, 15, 20, 25, 30, 50 or fewer conservative amino acid substitutions, to a reference amino acid sequence selected from the group consisting of SEQ ID NO: 8, SEQ ID NO: 12, SEQ ID NO: 16, SEQ ID NO: 41, SEQ ID NO: 44, and SEQ ID NO:45.

In a still further embodiment, the antibodies for use in the methods described herein comprise, consist essentially of, or consist of an immunoglobulin heavy chain variable region (VH) and an immunoglobulin light chain variable region (VL) identical, except for 1, 2, 3, 4, 5, 6, 7, 8, 9, 10, 15, 20, 25, 30, 50 or fewer conservative amino acid substitutions, to reference amino acid sequences selected from the group consisting of SEQ ID NO: 4 and SEQ ID NO: 8; SEQ ID NO: 6 and SEQ ID NO: 8; SEQ ID NO: 10 and SEQ ID NO: 12; SEQ ID NO: 14 and SEQ ID NO: 16; SEQ ID NO: 39 and SEQ ID NO: 41; SEQ ID NO: 42 and SEQ ID NO: 44; and SEQ ID NO: 43 and SEQ ID NO: 45.

In one embodiment, the antibody for use in the methods described herein is any one of the antibodies comprising an amino acid sequence of the V_{H} and/or V_{L} region as depicted in Tables 2 and 3. Alternatively, the antibody for use in the methods described herein is an antibody or antigen-binding fragment thereof, which competes for binding to Abeta with at least one of the antibodies having the V_{H} and/or V_{L} region as depicted in Tables 2 and 3. Those antibodies can be murine, humanized, xenogeneic, or chimeric human-murine antibodies. Humanized, xenogeneic, or chimeric human-murine antibodies can be particularly useful for therapeutic applications. For example, in one embodiment for use in the methods described herein, a chimeric human-mouse antibody can be used where the human IgG1 Fc region of a fully human antibody is replaced with a corresponding mouse IgG2a Fc region. An antigen-binding fragment of the antibody can be, for example, a single chain Fv fragment (scFv), a F(ab′) fragment, a F(ab) fragment, and an F(ab′)₂ fragment. For some applications only the variable regions of the antibodies are required, which can be obtained by treating the antibody with suitable reagents so as to generate Fab′, Fab, or F(ab″)₂ portions. Such fragments are useful, for example, in immunodiagnostic procedures involving coupling the immunospecific portion of an immunoglobulin to a detecting reagent such as a radioisotope, an inorganic molecule, or a peptide, using methods known in the art.

In one embodiment, the antibodies for use in the methods described herein comprise, consist essentially of, or consist of a Kabat heavy chain complementarity determining region-1 (VH-CDR1) amino acid sequence identical, except for five, four, three, two or fewer amino acid substitutions, to a reference VH-CDR1 amino acid sequence selected from the group consisting of: SEQ ID NO: 17, SEQ ID NO: 20, SEQ ID NO: 26, and SEQ ID NO: 32. In one embodiment, the VH-CDR1 amino acid sequence is selected from the group consisting of: SEQ ID NO: 17, SEQ ID NO: 20, SEQ ID NO: 26, and SEQ ID NO: 32.

In another embodiment, the antibodies for use in the methods described herein comprise, consist essentially of, or consist of a Kabat heavy chain complementarity determining region-2 (VH-CDR2) amino acid sequence identical, except for ten, nine, eight, seven, six, five, four or fewer amino acid substitutions, to a reference VH-CDR2 amino acid sequence selected from the group consisting of: SEQ ID NO: 18, SEQ ID NO: 21, SEQ ID NO: 27, and SEQ ID NO: 33. In one embodiment, the VH-CDR2 amino acid sequence is selected from the group consisting of: SEQ ID NO: 18, SEQ ID NO: 21, SEQ ID NO: 27, and SEQ ID NO: 33.

In a further embodiment, the antibodies for use in the methods described herein comprise, consist essentially of, or consist of a Kabat heavy chain complementarity determining region-3 (VH-CDR3) amino acid sequence identical, except for ten, nine, eight, seven, six, five, four or fewer amino acid substitutions, to a reference VH-CDR3 amino acid sequence selected from the group consisting of: SEQ ID NO: 19, SEQ ID NO: 22, SEQ ID NO: 28, and SEQ ID NO: 34. In one embodiment, the VH-CDR3 amino acid sequence is selected from the group consisting of: SEQ ID NO: 19, SEQ ID NO: 22, SEQ ID NO: 28, and SEQ ID NO: 34.

In another embodiment, the antibodies for use in the methods described herein comprise, consist essentially of, or consist of a Kabat light chain complementarity determining region-1 (VL-CDR1) amino acid sequence identical, except for ten, nine, eight, seven, six, five, four or fewer amino acid substitutions, to a reference VL-CDR1 amino acid sequence selected from the group consisting of: SEQ ID NO: 23, SEQ ID NO: 29, SEQ ID NO: 35, SEQ ID NO: 46, and SEQ ID NO: 49. In one embodiment, the VL-CDR1 amino acid sequence is selected from the group consisting of: SEQ ID NO: 23, SEQ ID NO: 29, SEQ ID NO: 35, SEQ ID NO: 46, and SEQ ID NO: 49.

In a further embodiment, the antibodies for use in the methods described herein comprise, consist essentially of, or consist of a Kabat light chain complementarity determining region-2 (VL-CDR2) amino acid sequence identical, except for five, four, three, two or fewer amino acid substitutions, to a reference VL-CDR2 amino acid sequence selected from the group consisting of: SEQ ID NO: 24, SEQ ID NO: 30, SEQ ID NO: 36, SEQ ID NO: 47, and SEQ ID NO: 50. In one embodiment, the VL-CDR2 amino acid sequence is selected from the group consisting of: SEQ ID NO: 24, SEQ ID NO: 30, SEQ ID NO: 36, SEQ ID NO: 47, and SEQ ID NO: 50.

In a still further embodiment, the antibodies for use in the methods described herein comprise, consist essentially of, or consist of a Kabat light chain complementarity determining region-3 (VL-CDR3) amino acid sequence identical, except for ten, nine, eight, seven, six, five, four or fewer amino acid substitutions, to a reference VL-CDR3 amino acid sequence selected from the group consisting of: SEQ ID NO: 25, SEQ ID NO: 31, SEQ ID NO: 37, SEQ ID NO: 48, and SEQ ID NO: 51. In one embodiment, the VL-CDR3 amino acid sequence is selected from the group consisting of: SEQ ID NO: 25, SEQ ID NO: 31, SEQ ID NO: 37, SEQ ID NO: 48, and SEQ ID NO: 51.

In another embodiment, the VH of the anti-Abeta antibody or antigen-binding fragment comprise, consist essentially of, or consist of VH-CDR1, VH-CDR2, and VH-CDR3 amino acid sequences selected from the group consisting of: SEQ ID NOs: 17, 18, and 19; SEQ ID NOs: 20, 21, and 22; SEQ ID NOs: 26, 27, and 28; and SEQ ID NOs: 32, 33, and 34, except for one, two, three, or four amino acid substitutions in at least one of the VH-CDRs. In one embodiment, the VH of the anti-Abeta antibody or antigen-binding fragment thereof comprises VH-CDR1, VH-CDR2, and VH-CDR3 amino acid sequences selected from the group consisting of: SEQ ID NOs: 17, 18, and 19; SEQ ID NOs: 20, 21, and 22; SEQ ID NOs: 26, 27, and 28; and SEQ ID NOs: 32, 33, and 34.

In another embodiment, the VL of the anti-Abeta antibody or antigen-binding fragment thereof comprises, consist essentially of, or consist of VL-CDR1, VL-CDR2, and VL-CDR3 amino acid sequences selected from the group consisting of: SEQ ID NOs: 23, 24, and 25; SEQ ID NOs: 29, 30, and 31; SEQ ID NOs: 35, 36, and 37; SEQ ID NOs: 46, 47 and 48; and SEQ ID NOs 49, 50 and 51, except for one, two, three, or four amino acid substitutions in at least one of the VL-CDRs. In one embodiment, the VL of the anti-Abeta antibody or antigen-binding fragment thereof comprises VL-CDR1, VL-CDR2, and VL-CDR3 amino acid sequences selected from the group consisting of: SEQ ID NOs: 23, 24, and 25; SEQ ID NOs: 29, 30, and 31; SEQ ID NOs: 35, 36, and 37; SEQ ID NOs: 46, 47 and 48; and SEQ ID NOs 49, 50 and 51.

In another embodiment, the methods described herein provide the use of an antibody comprising, consisting essentially of, or consisting of an immunoglobulin heavy chain variable region (VH), where at least one of VH-CDRs of the heavy chain variable region or at least two of the VH-CDRs of the heavy chain variable region are at least 80%, 85%, 90%, 95% or 100% identical to reference heavy chain VH-CDR1, VH-CDR2 or VH-CDR3 amino acid sequences from the antibodies disclosed herein. Alternatively, the VH-CDR1, VH-CDR2 and VH-CDR3 regions of the VH are at least 80%, 85%, 90%, 95% or 100% identical to reference heavy chain VH-CDR1, VH-CDR2 and VH-CDR3 amino acid sequences from the antibodies disclosed herein. Thus, according to this embodiment a heavy chain variable region has VH-CDR1, VH-CDR2 and VH-CDR3 polypeptide sequences related to the groups shown in Table 4, supra. While Table 4 shows VH-CDRs defined by the Kabat system, other CDR definitions, e.g., VH-CDRs defined by the Chothia system, are also contemplated, and can be easily identified by one of skill in the art using the data presented in Tables 2 and 3.

In another embodiment, the methods described herein provide the use of an antibody comprising, consisting essentially of, or consisting of an immunoglobulin heavy chain variable region (VH) in which the VH-CDR1, VH-CDR2 and VH-CDR3 regions have polypeptide sequences that are identical to the VH-CDR1, VH-CDR2 and VH-CDR3 groups shown in Table 4.

In another embodiment, the methods described herein provide the use of an antibody comprising, consisting essentially of, or consisting of an immunoglobulin heavy chain variable region (VH) in which the VH-CDR1, VH-CDR2 and VH-CDR3 regions have polypeptide sequences that are identical to the VH-CDR1, VH-CDR2 and VH-CDR3 groups shown in Table 4, except for one, two, three, four, five, or six amino acid substitutions in any one VH-CDR. In certain embodiments the amino acid substitutions are conservative.

In another embodiment, the methods described herein provide the use of an antibody comprising, consisting essentially of, or consisting of an immunoglobulin light chain variable region (VL), where at least one of the VL-CDRs of the light chain variable region or at least two of the VL-CDRs of the light chain variable region are at least 80%, 85%, 90% or 95% identical to reference light chain VL-CDR1, VL-CDR2 or VL-CDR3 amino acid sequences from antibodies disclosed herein. Alternatively, the VL-CDR1, VL-CDR2 and VL-CDR3 regions of the VL are at least 80%, 85%, 90% or 95% identical to reference light chain VL-CDR1, VL-CDR2 and VL-CDR3 amino acid sequences from antibodies disclosed herein. Thus, according to this embodiment a light chain variable region has VL-CDR1, VL-CDR2 and VL-CDR3 polypeptide sequences related to the polypeptides shown in Table 4, supra. While Table 4 shows VL-CDRs defined by the Kabat system, other CDR definitions, e.g., VL-CDRs defined by the Chothia system, are also contemplated.

In another embodiment, the methods described herein provide the use of an antibody comprising, consisting essentially of, or consisting of an immunoglobulin light chain variable region (VL) in which the VL-CDR1, VL-CDR2 and VL-CDR3 regions have polypeptide sequences that are identical to the VL-CDR1, VL-CDR2 and VL-CDR3 groups shown in Table 4.

In another embodiment, the methods described herein provide the use of an antibody comprising, consisting essentially of, or consisting of an immunoglobulin heavy chain variable region (VL) in which the VL-CDR1, VL-CDR2 and VL-CDR3 regions have polypeptide sequences that are identical to the VL-CDR1, VL-CDR2 and VL-CDR3 groups shown in Table 4, except for one, two, three, four, five, or six amino acid substitutions in any one VL-CDR. In certain embodiments the amino acid substitutions are conservative.

The present description is further directed to the use of the isolated polypeptides that are derived from an antibody for use in the methods described herein. Antibodies comprise polypeptides, e.g., amino acid sequences encoding specific antigen binding regions derived from immunoglobulin molecules. A polypeptide or amino acid sequence “derived from” a designated protein refers to the origin of the polypeptide having a certain amino acid sequence. In certain cases, the polypeptide or amino acid sequence that is derived from a particular starting polypeptide or amino acid sequence has an amino acid sequence that is essentially identical to that of the starting sequence, or a portion thereof, wherein the portion consists of at least 10-20 amino acids, at least 20-30 amino acids, at least 30-50 amino acids, or which is otherwise identifiable to one of skill in the art as having its origin in the starting sequence.

An immunoglobulin or its encoding cDNAs can be further modified. Thus, in a further embodiment the methods described herein can comprise any one of producing a chimeric antibody, humanized antibody, single-chain antibody, Fab-fragment, bi-specific antibody, fusion antibody, labeled antibody or an analog of any one of those. Corresponding methods are known to one of skill in the art and are described, e.g., in Harlow and Lane “Antibodies, A Laboratory Manual”, CSH Press, Cold Spring Harbor, 1988. For example, when derivatives of antibodies are obtained by the phage display technique, surface plasmon resonance as employed in the BIAcore system can be used to increase the efficiency of phage antibodies that bind to the same epitope as that of any one of the antibodies described herein (Schier, Human Antibodies Hybridomas 7 (1996), 97-105; Malmborg, J. Immunol. Methods 183 (1995), 7-13). The production of chimeric antibodies is described, for example, in international application WO89/09622. Methods for the WO89/09622. Methods for the production of humanized antibodies are described in, e.g., European application EP-A1 0 239 400 and international application WO90/07861. A further source of antibodies that can be utilized are so-called xenogeneic antibodies. The general principle for the production of xenogeneic antibodies such as human antibodies in mice is described in, e.g., international applications WO91/10741, WO94/02602, WO96/34096 and WO 96/33735. As discussed above, the antibody can exist in a variety of forms besides complete antibodies; including, for example, Fv, Fab and F(ab)₂, as well as in single chains; see e.g. international application WO88/09344.

The antibodies for use in the methods described herein or their corresponding immunoglobulin chain(s) can be further modified using conventional techniques known in the art, for example, by using amino acid deletion(s), insertion(s), substitution(s), addition(s), and/or recombination(s) and/or any other modification(s) known in the art either alone or in combination. Methods for introducing such modifications in the DNA sequence underlying the amino acid sequence of an immunoglobulin chain are well known to one of skill in the art; see, e.g., Sambrook, Molecular Cloning A Laboratory Manual, Cold Spring Harbor Laboratory (1989) N.Y. and Ausubel, Current Protocols in Molecular Biology, Green Publishing Associates and Wiley Interscience, N.Y. (1994).

Methods for the cloning of antibody variable regions and generation of recombinant antibodies are known to one of skill in the art and are described, for example, Gilliland et al., Tissue Antigens 47 (1996), 1-20; Doenecke et al., Leukemia 11 (1997), 1787-1792. Once the appropriate genetic material is obtained and, if desired, modified to encode an analog, the coding sequences, including those that encode, at a minimum, the variable regions of the heavy and light chain, can be inserted into expression systems contained on vectors that can be transfected into standard recombinant host cells. A variety of such host cells can be used, for example mammalian cells can be used for efficient processing. Typical mammalian cell lines useful for this purpose include, but are not limited to, CHO cells, HEK 293 cells, or NSO cells.

The production of the antibody or analog is then undertaken by culturing the modified recombinant host under culture conditions appropriate for the growth of the host cells and the expression of the coding sequences. The antibodies are then recovered by isolating them from the culture. The expression systems can be designed to include signal peptides so that the resulting antibodies are secreted into the medium; however, intracellular production is also possible.

Modifications of the antibody include chemical and/or enzymatic derivatizations at one or more constituent amino acids, including side chain modifications, backbone modifications, and N- and C-terminal modifications including acetylation, hydroxylation, methylation, amidation, and the attachment of carbohydrate or lipid moieties, cofactors, and the like. Likewise, the present description encompasses the production of chimeric proteins that comprise the described antibody or some fragment thereof at the amino terminus fused to heterologous molecule such as an immunostimulatory ligand at the carboxyl terminus; see, e.g., international application WO00/30680 for corresponding technical details.

Additionally, the present description encompasses the use of small peptides in the methods described herein, including those containing an Abeta binding molecule as described above, for example containing the CDR3 region of the variable region of any one of the mentioned antibodies, in particular CDR3 of the heavy chain since it has frequently been observed that heavy chain CDR3 (HCDR3) is the region having a greater degree of variability and a predominant participation in antigen-antibody interaction. Such peptides can easily be synthesized or produced by recombinant means to produce a binding agent. Such methods are well known to one of skill in the art. Peptides can be synthesized for example, using automated peptide synthesizers that are commercially available. The peptides can be produced by recombinant techniques by incorporating the DNA expressing the peptide into an expression vector and transforming cells with the expression vector to produce the peptide.

In accordance with the above, the present description also relates to the use of a polynucleotide encoding the antigen or Abeta binding molecule in the methods described herein, in case of the antibody at least one variable region of an immunoglobulin chain of the antibody described above can be used. Typically, said variable region encoded by the polynucleotide comprises at least one complementarity determining region (CDR) of the V_{H} and/or V_{L} of the variable region of the said antibody. The person skilled in the art knows that each variable domain (the heavy chain V_{H} and light chain V_{L}) of an antibody comprises three hypervariable regions, sometimes called complementarity determining regions or “CDRs” flanked by four relatively conserved framework regions or “FRs” and refer to the amino acid residues of an antibody that are responsible for antigen-binding. The hypervariable regions or CDRs of the human IgG subtype of antibody comprise amino acid residues from residues 24-34 (L1), 50-56 (L2) and 89-97 (L3) in the light chain variable domain and 31-35 (H1), 50-65 (H2) and 95-102 (H3) in the heavy chain variable domain as described by Kabat et al., Sequences of Proteins of Immunological Interest, 5th Ed Public Health Service, National Institutes of Health, Bethesda, Md. (1991) and/or those residues from a hypervariable loop, e.g. residues 26-32 (L1), 50-52 (L2) and 91-96 (L3) in the light chain variable domain and 26-32 (H1), 53-55 (H2) and 96-101 (H3) in the heavy chain variable domain as described by Chothia et al., J. Mol. Biol. 196 (1987), 901-917. Framework or FR residues are those variable domain residues other than and bracketing the hypervariable regions. The term “specific binding” refers to antibody binding to a predetermined antigen. Typically, the antibody binds with a dissociation constant (K_{D}) of 10⁻⁷ M or less, and binds to the predetermined antigen with a K_{D} that is at least twofold less than its K_{D} for binding to a nonspecific antigen (e.g., BSA, casein, or any other specified polypeptide) other than the predetermined antigen. The phrases “an antibody recognizing an antigen” and “an antibody specific for an antigen” are used interchangeably herein with the term “an antibody that binds specifically to an antigen”. As used herein “highly specific” binding means that the relative K_{D} of the antibody for the specific target epitope is at least 10-fold less than the K_{D} for binding that antibody to other ligands.

The affinity or avidity of an antibody for an antigen can be determined experimentally using any suitable method; see, for example, Berzofsky et al., “Antibody-Antigen Interactions” In Fundamental Immunology, Paul, W. E., Ed., Raven Press New York, N.Y. (1984), Kuby, Janis Immunology, W. H. Freeman and Company New York, N.Y. (1992), and methods described herein. General techniques for measuring the affinity of an antibody for an antigen include ELISA, RIA, and surface plasmon resonance. The measured affinity of a particular antibody-antigen interaction can vary if measured under different conditions, e.g., salt concentration, pH. Thus, measurements of affinity and other antigen-binding parameters, e.g., K_{D}, IC₅₀, can be made with standardized solutions of antibody and antigen, and a standardized buffer.

One of skill in the art will readily appreciate that the variable domain of the antibody having the above-described variable domain can be used for the construction of other polypeptides or antibodies of desired specificity and biological function. Thus, the present description also encompasses polypeptides and antibodies comprising at least one CDR of the above-described variable domain and which advantageously have substantially the same or similar binding properties as the antibody described in the appended examples. One of skill in the art will readily appreciate that using the variable domains or CDRs described herein antibodies can be constructed according to methods known in the art, e.g., as described in European patent applications EP 0 451 216 A1 and EP 0 549 581 A1. Furthermore, one of skill in the art knows that binding affinity can be enhanced by making amino acid substitutions within the CDRs or within the hypervariable loops (Chothia and Lesk, J. Mol. Biol. 196 (1987), 901-917) that partially overlap with the CDRs as defined by Kabat. Thus, the present description also relates to antibodies wherein one or more of the mentioned CDRs comprise one or more, or not more than two amino acid substitutions. The antibody can comprise in one or both of its immunoglobulin chains two or all three CDRs of the variable regions as set forth in Table 4.

Abeta binding molecules, e.g., antibodies, or antigen-binding fragments, variants, or derivatives thereof, as known by one of skill in the art, can comprise a constant region that mediates one or more effector functions. For example, binding of the C1 component of complement to an antibody constant region can activate the complement system. Activation of complement is important in the opsonisation and lysis of cell pathogens. The activation of complement also stimulates the inflammatory response and can also be involved in autoimmune hypersensitivity. Further, antibodies bind to receptors on various cells via the Fc region, with a Fc receptor binding site on the antibody Fc region binding to a Fc receptor (FcR) on a cell. There are a number of Fc receptors that are specific for different classes of antibody, including IgG (gamma receptors), IgE (epsilon receptors), IgA (alpha receptors) and IgM (mu receptors). Binding of antibody to Fc receptors on cell surfaces triggers a number of important and diverse biological responses including engulfment and destruction of antibody-coated particles, clearance of immune complexes, lysis of antibody-coated target cells by killer cells (called antibody-dependent cell-mediated cytotoxicity, or ADCC), release of inflammatory mediators, placental transfer and control of immunoglobulin production.

Accordingly, certain embodiments for use in the methods described herein include an antibody, or antigen-binding fragment, variant, or derivative thereof, in which at least a fraction of one or more of the constant region domains has been deleted or otherwise altered so as to provide desired biochemical characteristics such as reduced effector functions, the ability to non-covalently dimerize, increased ability to localize at a target site, reduced serum half-life, or increased serum half-life when compared with a whole, unaltered antibody of approximately the same immunogenicity. For example, certain antibodies for use in the diagnostic and treatment methods described herein are domain deleted antibodies that comprise a polypeptide chain similar to an immunoglobulin heavy chain, but which lack at least a portion of one or more heavy chain domains. For instance, in certain antibodies, one entire domain of the constant region of the modified antibody will be deleted, for example, all or part of the CH2 domain will be deleted. In other embodiments, certain antibodies for use in the diagnostic and treatment methods described herein have a constant region, e.g., an IgG heavy chain constant region, which is altered to eliminate glycosylation, referred to elsewhere herein as aglycosylated or “agly” antibodies. Such “agly” antibodies can be prepared enzymatically as well as by engineering the consensus glycosylation site(s) in the constant region. While not being bound by theory, it is believed that “agly” antibodies can have an improved safety and stability profile in vivo. Methods of producing aglycosylated antibodies, having desired effector function are found for example in WO 2005/018572, which is incorporated by reference in its entirety.

In certain antibodies, or antigen-binding fragments, variants, or derivatives thereof described herein, the Fc portion can be mutated to decrease effector function using techniques known in the art. For example, the deletion or inactivation (through point mutations or other means) of a constant region domain can reduce Fc receptor binding of the circulating modified antibody thereby increasing localization to specific targets. In other cases it can be that constant region modifications moderate complement binding and thus reduce the serum half life and nonspecific association of a conjugated cytotoxin. Yet other modifications of the constant region can be used to modify disulfide linkages or oligosaccharide moieties that allow for enhanced localization due to increased antigen specificity or antibody flexibility. The resulting physiological profile, bioavailability and other biochemical effects of the modifications, such as localization, biodistribution and serum half-life, can easily be measured and quantified using well know immunological techniques without undue experimentation.

Modified forms of antibodies, or antigen-binding fragments, variants, or derivatives thereof can be made from whole precursor or parent antibodies using techniques known in the art. Exemplary techniques are discussed in more detail herein.

In certain embodiments both the variable and constant regions of the antibodies, or antigen-binding fragments, variants, or derivatives thereof are fully human. Fully human antibodies can be made using techniques that are known in the art and as described herein. For example, fully human antibodies against a specific antigen can be prepared by administering the antigen to a transgenic animal that has been modified to produce such antibodies in response to antigenic challenge, but whose endogenous loci have been disabled. Exemplary techniques that can be used to make such antibodies are described in U.S. Pat. Nos. 6,150,584; 6,458,592; 6,420,140. Other techniques are known in the art. Fully human antibodies can likewise be produced by various display technologies, e.g., phage display or other viral display systems, as known in the art.

Antibodies, or antigen-binding fragments, variants, or derivatives thereof can be made or manufactured using techniques that are known in the art. In certain embodiments, antibody molecules or fragments thereof are “recombinantly produced,” i.e., are produced using recombinant DNA technology. Exemplary techniques for making antibody molecules or fragments thereof are discussed in more detail elsewhere herein.

Antibodies, or antigen-binding fragments, variants, or derivatives thereof also include derivatives that are modified, e.g., by the covalent attachment of any type of molecule to the antibody such that covalent attachment does not prevent the antibody from specifically binding to its cognate epitope. For example, but not by way of limitation, the antibody derivatives include antibodies that have been modified, e.g., by glycosylation, acetylation, pegylation, phosphorylation, amidation, derivatization by known protecting/blocking groups, proteolytic cleavage, linkage to a cellular ligand or other protein, etc. Any of numerous chemical modifications may be carried out by known techniques, including, but not limited to specific chemical cleavage, acetylation, formylation, or metabolic synthesis of tunicamycin. The derivative can contain one or more non-classical amino acids.

In certain embodiments, antibodies, or antigen-binding fragments, variants, or derivatives thereof will not elicit a deleterious immune response in the animal to be treated, e.g., in a human. In certain embodiments, an Abeta binding molecule, e.g., antibody, or antigen-binding fragment thereof, is derived from a subject, e.g., a human patient, and is subsequently used in the same species from which it was derived, e.g., human, alleviating or minimizing the occurrence of deleterious immune responses.

De-immunization can also be used to decrease the immunogenicity of an antibody. As used herein, the term “de-immunization” includes alteration of an antibody to modify T cell epitopes (see, e.g., WO9852976A1, WO0034317A2). For example, VH and VL sequences from the starting antibody are analyzed and a human T cell epitope “map” from each V region showing the location of epitopes in relation to complementarity-determining regions (CDRs) and other key residues within the sequence. Individual T cell epitopes from the T cell epitope map are analyzed in order to identify alternative amino acid substitutions with a low risk of altering activity of the final antibody. A range of alternative VH and VL sequences are designed comprising combinations of amino acid substitutions and these sequences are subsequently incorporated into a range of binding polypeptides, e.g., neo-epitope-specific antibodies or immunospecific fragments thereof for use in the diagnostic and treatment methods disclosed herein, which are then tested for function. Typically, between 12 and 24 variant antibodies are generated and tested. Complete heavy and light chain genes comprising modified V and human C regions are then cloned into expression vectors and the subsequent plasmids introduced into cell lines for the production of whole antibody. The antibodies are then compared in appropriate biochemical and biological assays, and the optimal variant is identified.

Monoclonal antibodies can be prepared using techniques known in the art including the use of hybridoma, recombinant, and phage display technologies, or a combination thereof. For example, monoclonal antibodies can be produced using hybridoma techniques known in the art and taught, for example, in Harlow et al., Antibodies: A Laboratory Manual, Cold Spring Harbor Laboratory Press, 2nd ed. (1988); Hammerling et al., in: Monoclonal Antibodies and T-Cell Hybridomas Elsevier, N.Y., 563-681 (1981) (said references incorporated by reference in their entireties). The term “monoclonal antibody” as used herein is not limited to antibodies produced through hybridoma technology. The term “monoclonal antibody” refers to an antibody that is derived from a single clone, including any eukaryotic, prokaryotic, or phage clone, and not the method by which it is produced. Thus, the term “monoclonal antibody” is not limited to antibodies produced through hybridoma technology. Monoclonal antibodies can be prepared using a wide variety of techniques known in the art. In certain embodiments, antibodies are derived from human B cells that have been immortalized via transformation with Epstein-Barr virus, as described herein.

Antibody fragments that recognize specific epitopes can be generated by known techniques. For example, Fab and F(ab′)₂ fragments can be produced recombinantly or by proteolytic cleavage of immunoglobulin molecules, using enzymes such as papain (to produce Fab fragments) or pepsin (to produce F(ab′)₂ fragments). F(ab′)₂ fragments contain the variable region, the light chain constant region and the CH1 domain of the heavy chain.

Completely human antibodies, such as described herein, are particularly desirable for therapeutic treatment of human patients. Human antibodies can be made by a variety of methods known in the art including phage display methods described above using antibody libraries derived from human immunoglobulin sequences. See also, U.S. Pat. Nos. 4,444,887 and 4,716,111; and PCT publications WO 98/46645, WO 98/50433, WO 98/24893, WO 98/16654, WO 96/34096, WO 96/33735, and WO 91/10741; each of which is incorporated herein by reference in its entirety. Human antibodies can be isolated, e.g., from a subject who is symptom free but is at risk of developing a disorder, e.g., Alzheimer's disease, or a patient diagnosed with the disorder but with an unusually stable disease course.

In another embodiment, DNA encoding desired monoclonal antibodies can be readily isolated and sequenced using conventional procedures (e.g., by using oligonucleotide probes that are capable of binding specifically to genes encoding the heavy and light chains of murine antibodies). The isolated and subcloned hybridoma cells serve as a source of such DNA. Once isolated, the DNA can be placed into expression vectors, which are then transfected into prokaryotic or eukaryotic host cells such as, but not limited to, E. coli cells, simian COS cells, Chinese Hamster Ovary (CHO) cells or myeloma cells that do not otherwise produce immunoglobulins. More particularly, the isolated DNA (which can be synthetic as described herein) can be used to clone constant and variable region sequences for the manufacture antibodies as described in Newman et al., U.S. Pat. No. 5,658,570, filed Jan. 25, 1995, which is incorporated by reference herein. Essentially, this entails extraction of RNA from the selected cells, conversion to cDNA, and amplification by PCR using Ig specific primers. Suitable primers for this purpose are also described in U.S. Pat. No. 5,658,570. As will be discussed in more detail below, transformed cells expressing the desired antibody can be grown up in relatively large quantities to provide clinical and commercial supplies of the immunoglobulin.

In one embodiment, an antibody for use in the methods described herein comprises at least one heavy or light chain CDR of an antibody molecule. In another embodiment, an antibody for use in the methods described herein comprises at least two CDRs from one or more antibody molecules. In another embodiment, an antibody for use in the methods described herein comprises at least three CDRs from one or more antibody molecules. In another embodiment, an antibody for use in the methods described herein comprises at least four CDRs from one or more antibody molecules. In another embodiment, an antibody for use in the methods described herein comprises at least five CDRs from one or more antibody molecules. In another embodiment, an antibody for use in the methods described herein comprises at least six CDRs from one or more antibody molecules. Exemplary antibody molecules comprising at least one CDR that can be included in the subject antibodies are described herein.

In a specific embodiment, the amino acid sequence of the heavy and/or light chain variable domains can be inspected to identify the sequences of the complementarity determining regions (CDRs) by methods that are well know in the art, e.g., by comparison to known amino acid sequences of other heavy and light chain variable regions to determine the regions of sequence hypervariability. Using routine recombinant DNA techniques, one or more of the CDRs can be inserted within framework regions, e.g., into human framework regions. The framework regions can be naturally occurring or consensus framework regions, or human framework regions (see, e.g., Chothia et al., J. Mol. Biol. 278:457-479 (1998) for a listing of human framework regions). In certain embodiments, the polynucleotide generated by the combination of the framework regions and CDRs encodes an antibody that specifically binds to at least one epitope of a desired polypeptide. In certain embodiments, one or more amino acid substitutions can be made within the framework regions, to, e.g., improve binding of the antibody to its antigen. Additionally, such methods can be used to make amino acid substitutions or deletions of one or more variable region cysteine residues participating in an intrachain disulfide bond to generate antibody molecules lacking one or more intrachain disulfide bonds. Other alterations to the polynucleotide are contemplated and known by one of skill of the art.

Alternatively, techniques described for the production of single chain antibodies (U.S. Pat. No. 4,694,778; Bird, Science 242:423-442 (1988); Huston et al., Proc. Natl. Acad. Sci. USA 85:5879-5883 (1988); and Ward et al., Nature 334:544-554 (1989)) can be adapted to produce single chain antibodies. Single chain antibodies are formed by linking the heavy and light chain fragments of the Fv region via an amino acid bridge, resulting in a single chain antibody. Techniques for the assembly of functional Fv fragments in E coli can also be used (Skerra et al., Science 242:1038-1041 (1988)).

In another embodiment, lymphocytes can be selected by micromanipulation and the variable genes isolated. For example, peripheral blood mononuclear cells can be isolated from an immunized or naturally immune mammal, e.g., a human, and cultured for about 7 days in vitro. The cultures can be screened for specific IgGs that meet the screening criteria. Cells from positive wells can be isolated. Individual Ig-producing B cells can be isolated by FACS or by identifying them in a complement-mediated hemolytic plaque assay. Ig-producing B cells can be micromanipulated into a tube and the VH and VL genes can be amplified using, e.g., RT-PCR. The VH and VL genes can be cloned into an antibody expression vector and transfected into cells (e.g., eukaryotic or prokaryotic cells) for expression.

Alternatively, antibody-producing cell lines can be selected and cultured using techniques well known to one of skill in the art. Such techniques are described in a variety of laboratory manuals and primary publications. In this respect, techniques suitable for use in the methods as described below are described in Current Protocols in Immunology, Coligan et al., Eds., Green Publishing Associates and Wiley-Interscience, John Wiley and Sons, New York (1991), which is herein incorporated by reference in its entirety, including supplements.

Antibodies for use in the methods described herein can be produced by any method known in the art for the synthesis of antibodies, in particular, by chemical synthesis by recombinant expression techniques as described herein.

In one embodiment, an antibody, or antigen-binding fragment, variant, or derivative thereof for use in the methods described herein comprises a synthetic constant region wherein one or more domains are partially or entirely deleted (“domain-deleted antibodies”). In certain embodiments compatible modified antibodies will comprise domain deleted constructs or variants wherein the entire CH2 domain has been removed (ΔCH2 constructs). For other embodiments a short connecting peptide can be substituted for the deleted domain to provide flexibility and freedom of movement for the variable region. One of skill in the art will appreciate that such constructs are useful due to the regulatory properties of the CH2 domain on the catabolic rate of the antibody. Domain deleted constructs can be derived using a vector encoding an IgG₁ human constant domain (see, e.g., WO 02/060955A2 and WO02/096948A2). This vector is engineered to delete the CH2 domain and provide a synthetic vector expressing a domain deleted IgG₁ constant region.

In certain embodiments, antibodies, or antigen-binding fragments, variants, or derivatives thereof for use in the methods described herein are minibodies. Minibodies can be made using methods described in the art (see, e.g., U.S. Pat. No. 5,837,821 or WO 94/09817A1).

In one embodiment, an antibody, or antigen-binding fragment, variant, or derivative thereof for use in the methods described herein comprises an immunoglobulin heavy chain having deletion or substitution of at least one amino acid as long as it permits association between the monomeric subunits. For example, the mutation of a single amino acid in selected areas of the CH2 domain can substantially reduce Fc binding and thereby increase target tissue localization. Similarly, it can be desirable to delete that part of one or more constant region domains that control the effector function (e.g., complement binding) to be modulated. Such partial deletions of the constant regions can improve selected characteristics of the antibody (such as serum half-life) while leaving a desirable function associated with the subject constant region domain intact. Moreover, as alluded to above, the constant regions of the disclosed antibodies can be synthetic through the mutation or substitution of one or more amino acids that enhances the immunogenic profile of the resulting construct. In this respect it can be possible to disrupt the activity provided by a conserved binding site (e.g., Fc binding) while substantially maintaining the configuration and immunogenic profile of the modified antibody. Yet other embodiments comprise the addition of one or more amino acids to the constant region to enhance desirable characteristics such as effector function or provide for more cytotoxin or carbohydrate attachment. In such embodiments it can be desirable to insert or replicate specific sequences derived from selected constant region domains.

The present description also provides antibodies for use in the methods described herein that comprise, consist essentially of, or consist of, variants (including derivatives) of antibody molecules (e.g., the VH regions and/or VL regions) described herein, which antibodies or fragments thereof immunospecifically bind an Abeta. Techniques known to one of skill in the art can be used to introduce mutations in the nucleotide sequence encoding an antibody, including, but not limited to, site-directed mutagenesis and PCR-mediated mutagenesis that result in amino acid substitutions. The variants (including derivatives) can encode less than 50 amino acid substitutions, less than 40 amino acid substitutions, less than 30 amino acid substitutions, less than 25 amino acid substitutions, less than 20 amino acid substitutions, less than 15 amino acid substitutions, less than 10 amino acid substitutions, less than 5 amino acid substitutions, less than 4 amino acid substitutions, less than 3 amino acid substitutions, or less than 2 amino acid substitutions relative to the reference VH region, VH-CDR1, VH-CDR2, VH-CDR3, VL region, VL-CDR1, VL-CDR2, or VL-CDR3. A “conservative amino acid substitution” is one in which the amino acid residue is replaced with an amino acid residue having a side chain with a similar charge. Families of amino acid residues having side chains with similar charges have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Alternatively, mutations can be introduced randomly along all or part of the coding sequence, such as by saturation mutagenesis, and the resultant mutants can be screened for biological activity to identify mutants that retain activity (e.g., the ability to bind an disorder-associated polypeptide).

For example, it is possible to introduce mutations only in framework regions or only in CDR regions of an antibody molecule. Introduced mutations may be silent or neutral missense mutations, e.g., have no, or little, effect on an antibody's ability to bind antigen, indeed some such mutations do not alter the amino acid sequence whatsoever. These types of mutations can be useful to optimize codon usage, or improve a hybridoma's antibody production. Codon-optimized coding regions encoding antibodies are disclosed elsewhere herein. Alternatively, non-neutral missense mutations can alter an antibody's ability to bind antigen. The location of most silent and neutral missense mutations is likely to be in the framework regions, while the location of most non-neutral missense mutations is likely to be in CDR, though this is not an absolute requirement. One of skill in the art would be able to design and test mutant molecules with desired properties such as no alteration in antigen binding activity or alteration in binding activity (e.g., improvements in antigen binding activity or change in antibody specificity). Following mutagenesis, the encoded protein can routinely be expressed and the functional and/or biological activity of the encoded protein, (e.g., ability to immunospecifically bind at least one epitope of a disorder-associated polypeptide) can be determined using techniques described herein or by routinely modifying techniques known in the art.

IV. Polynucleotides Encoding Antibodies

In accordance with the above, the present description also relates to the use of a polynucleotide encoding an Abeta binding molecule, e.g., an antibody in the methods described herein. In the case of the antibody, the polynucleotide can encode at least a variable region of an immunoglobulin chain of the antibody described above. The polynucleotide encoding the above described antibody can be, e.g., DNA, cDNA, RNA or synthetically produced DNA or RNA or a recombinantly produced chimeric nucleic acid molecule comprising any of those polynucleotides either alone or in combination. The polynucleotide can be part of a vector. Such vectors can comprise further genes such as marker genes that allow for the selection of said vector in a suitable host cell and under suitable conditions. The polynucleotide can be operatively linked to expression control sequences allowing expression in prokaryotic or eukaryotic cells. Expression of said polynucleotide comprises transcription of the polynucleotide into a translatable mRNA. Regulatory elements ensuring expression in eukaryotic cells, such as mammalian cells, are well known to one of skill in the art. They usually comprise regulatory sequences ensuring initiation of transcription and optionally poly-A signals ensuring termination of transcription and stabilization of the transcript. Additional regulatory elements can include transcriptional as well as translational enhancers, and/or naturally associated or heterologous promoter regions.

A polynucleotide encoding an antibody, or antigen-binding fragment, variant, or derivative thereof can be composed of any polyribonucleotide or polydeoxyribonucleotide, that can be unmodified RNA or DNA or modified RNA or DNA. For example, a polynucleotide encoding an antibody, or antigen-binding fragment, variant, or derivative thereof can be composed of single- and double-stranded DNA, DNA that is a mixture of single- and double-stranded regions, single- and double-stranded RNA, and RNA that is mixture of single- and double-stranded regions, hybrid molecules comprising DNA and RNA that can be single-stranded or, more typically, double-stranded or a mixture of single- and double-stranded regions. In addition, a polynucleotide encoding an antibody, or antigen-binding fragment, variant, or derivative thereof can be composed of triple-stranded regions comprising RNA or DNA or both RNA and DNA. A polynucleotide encoding an antibody, or antigen-binding fragment, variant, or derivative thereof can also contain one or more modified bases or DNA or RNA backbones modified for stability or for other reasons. “Modified” bases include, for example, tritylated bases and unusual bases such as inosine. A variety of modifications can be made to DNA and RNA; thus, “polynucleotide” embraces chemically, enzymatically, or metabolically modified forms.

An isolated polynucleotide encoding a non-natural variant of a polypeptide derived from an immunoglobulin (e.g., an immunoglobulin heavy chain portion or light chain portion) can be created by introducing one or more nucleotide substitutions, additions or deletions into the nucleotide sequence of the immunoglobulin such that one or more amino acid substitutions, additions or deletions are introduced into the encoded protein. Mutations can be introduced by standard techniques, such as site-directed mutagenesis and PCR-mediated mutagenesis. Conservative amino acid substitutions can be made at one or more non-essential amino acid residues.

As is known, RNA can be isolated from the original hybridoma cells or from other transformed cells by standard techniques, such as guanidinium isothiocyanate extraction and precipitation followed by centrifugation or chromatography. Where desirable, mRNA can be isolated from total RNA by techniques such as chromatography on oligo dT cellulose. Suitable techniques are familiar in the art.

In one embodiment, cDNAs that encode the light and the heavy chains of the antibody can be made, either simultaneously or separately, using reverse transcriptase and DNA polymerase in accordance with well known methods. PCR can be initiated by consensus constant region primers or by more specific primers based on the published heavy and light chain DNA and amino acid sequences. As discussed above, PCR also can be used to isolate DNA clones encoding the antibody light and heavy chains. In this case the libraries can be screened by consensus primers or larger homologous probes, such as mouse constant region probes.

DNA, typically plasmid DNA, can be isolated from the cells using techniques known in the art, restriction mapped and sequenced in accordance with standard, well known techniques set forth in detail, e.g., in the foregoing references relating to recombinant DNA techniques. Of course, the DNA can be synthetic at any point during the isolation process or subsequent analysis.

In one embodiment, an isolated polynucleotide comprises, consists essentially of, or consists of a nucleic acid encoding an immunoglobulin heavy chain variable region (VH), where at least one of the CDRs of the heavy chain variable region or at least two of the VH-CDRs of the heavy chain variable region are at least 80%, 85%, 90%, or 95% identical to reference heavy chain VH-CDR1, VH-CDR2, or VH-CDR3 amino acid sequences from the antibodies disclosed herein. Alternatively, the VH-CDR1, VH-CDR2, and VH-CDR3 regions of the VH are at least 80%, 85%, 90%, or 95% identical to reference heavy chain VH-CDR1, VH-CDR2, and VH-CDR3 amino acid sequences from the antibodies disclosed herein. Thus, according to this embodiment a heavy chain variable region has VH-CDR1, VH-CDR2, or VH-CDR3 polypeptide sequences related to the polypeptide sequences shown in Table 4.

In another embodiment, an isolated polynucleotide comprises, consists essentially of, or consists of a nucleic acid encoding an immunoglobulin light chain variable region (VL), where at least one of the VL-CDRs of the light chain variable region or at least two of the VL-CDRs of the light chain variable region are at least 80%, 85%, 90%, or 95% identical to reference light chain VL-CDR1, VL-CDR2, or VL-CDR3 amino acid sequences from the antibodies disclosed herein. Alternatively, the VL-CDR1, VL-CDR2, and VL-CDR3 regions of the VL are at least 80%, 85%, 90%, or 95% identical to reference light chain VL-CDR1, VL-CDR2, and VL-CDR3 amino acid sequences from the antibodies disclosed herein. Thus, according to this embodiment a light chain variable region has VL-CDR1, VL-CDR2, or VL-CDR3 polypeptide sequences related to the polypeptide sequences shown in Table 4.

In another embodiment, an isolated polynucleotide comprises, consists essentially of, or consists of a nucleic acid encoding an immunoglobulin heavy chain variable region (VH) in which the VH-CDR1, VH-CDR2, and VH-CDR3 regions have polypeptide sequences that are identical to the VH-CDR1, VH-CDR2, and VH-CDR3 groups shown in Table 4.

As known in the art, “sequence identity” between two polypeptides or two polynucleotides is determined by comparing the amino acid or nucleic acid sequence of one polypeptide or polynucleotide to the sequence of a second polypeptide or polynucleotide. When discussed herein, whether any particular polypeptide is at least about 40%, 45%, 50%, 55%, 60%, 65%, 70%, 75%, 80%, 85%, 90% or 95% identical to another polypeptide can be determined using methods and computer programs/software known in the art such as, but not limited to, the BESTFIT program (Wisconsin Sequence Analysis Package, Version 8 for Unix, Genetics Computer Group, University Research Park, 575 Science Drive, Madison, Wis. 53711). BESTFIT uses the local homology algorithm of Smith and Waterman, Advances in Applied Mathematics 2:482-489 (1981), to find the best segment of homology between two sequences. When using BESTFIT or any other sequence alignment program to determine whether a particular sequence is, for example, 95% identical to a reference sequence, the parameters are set, of course, such that the percentage of identity is calculated over the full length of the reference polypeptide sequence and that gaps in homology of up to 5% of the total number of amino acids in the reference sequence are allowed.

TABLE 5
Polynucleotide sequences of the V_{H} region.

| Antibody | Variable heavy chain sequence |
|---|---|
| NI-101.10 | GAGGTGCAGCTAGTGCAGTCTGGGGGAGGC |
| (SEQ ID NO: 3) | GTGGTCCAGCCTGGGAGGTCCCTGAGACTC |
| | TCCTGTGCAGCGTCTGGATTCGCCTTCAGT |
| | AGCTATGGCATACACTGGGTCCGCCAGGCT |
| | CCAGGCAAGGGGCTGGAGTGGGTGGCAGTT |
| | ATATGGTTTGATGGAACTAAAAAATACTAT |
| | ACAGACTCCGTGAAGGGCAGATTCACCATC |
| | TCCAGAGACAATTCCAAGAACACACTGTAT |
| | CTGCAAATGAACACCCTGAGAGCCGAGGAC |
| | ACGGCTGTGTATTACTGTGCGAGAGATAGG |
| | GGTATAGGAGCTCGGCGGGGGCCGTACTAC |
| | ATGGACGTCTGGGGCAAAGGGACCACGGTC |
| | ACCGTCTCCTCA |
| NI-101.11 | GAGGTGCAGCTGGTGCAGTCTGGGGGAGGC |
| (SEQ ID NO: 1) | GTGGTCCAGCCTGGGAGGTCCCTGAGACTC |
| | TCCTGTGCAGCGTCTGGATTCGCCTTCAGT |
| | AGCTATGGCATGCACTGGGTCCGCCAGGCT |
| | CCAGGCAAGGGGCTGGAGTGGGTGGCAGTT |
| | ATATGGTTTGATGGAACTAAAAAATACTAT |
| | ACAGACTCCGTGAAGGGCAGATTCACCATC |
| | TCCAGAGACAATTCCAAGAACACACTGTAT |
| | CTGCAAATGAACACCCTGAGAGCCGAGGAC |
| | ACGGCTGTGTATTACTGTGCGAGAGATAGG |
| | GGTATAGGAGCTCGGCGGGGGCCGTACTAC |
| | ATGGACGTCTGGGGCAAAGGGACCACGGTC |
| | ACCGTCTCCTCA |
| NI-101.11 | GAGGTGCAGCTGGTGCAGAGCGGCGGCGGC |
| (SEQ ID NO: 5) | GTGGTGCAGCCCGGCCGGAGCCTGCGGCTG |
| (codon- | AGCTGCGCCGCCAGCGGCTTCGCCTTCAGC |
| optimized) | AGCTACGGCATGCACTGGGTGCGGCAGGCC |
| | CCCGGCAAGGGCCTGGAGTGGGTGGCCGTG |
| | ATCTGGTTCGACGGCACCAAGAAGTACTAC |
| | ACCGACAGCGTGAAGGGCCGGTTCACCATC |
| | AGCCGGGACAACAGCAAGAACACCCTGTAC |
| | CTGCAGATGAACACCCTGCGGGCCGAGGAC |
| | ACCGCCGTGTACTACTGCGCCCGGGACCGG |
| | GGCATCGGCGCCCGGCGGGGCCCCTACTAC |
| | ATGGACGTGTGGGGCAAGGGCACCACCGTG |
| | ACCGTGAGCAGC |
| NI-101.12 | GAGGTGCAGCTGGTGGAGAGCGGCCCCGGC |
| (SEQ ID NO: 9) | CTGGTGAAGCCCGCCGAGACCCTGAGCCTG |
| | ACCTGCACCGTGAGCGGCGGCAGCATCCGG |
| | AGCGGCAGCATCTGCTGGTACTGGATCCGG |
| | CAGCCCCCCGGCAAGGGCCTGGAGTGGATC |
| | GGCTACTTCTGCTACAGCGGCGCCACCTTC |
| | TACACCCCCAGCCTGCGGGGCCGGCTGACC |
| | ATCAGCGTGGACGCCAGCAAGAACCAGCTG |
| | AGCCTGAGCCTGAGCAGCGTGACCGCCGCC |
| | GACACCGCCGTGTACTACTGCGCCCGGCGG |
| | GCCGGCGAGAACAGCGGCGGCATCGAGCCC |
| | TACTACGGCATGGACGTGTGGGGCCAGGGC |
| | ACCACCGTGACCGTGAGCAGC |
| NI-101.13 | CAGGTACAGCTGCAGGAGTCAGGCCCAGGAC |
| (SEQ ID NO: 13) | TGGTGAAGCCTTCGGAGACCCTGTCCCTCAC |
| | CTGCACTGTCTCTGGTGGCTCCATCAGCAGA |
| | AGAAGTTACTACTGGGGCTGGATCCGCCAGT |
| | CCCCAGGGAAGGGGCTGGAGTGGAGTGGAAG |
| | TATCCATTATAGCGGGAGCACCTACTACAAC |
| | CCGTCCCTCAAGAGTCGAGTCACCATATCTG |
| | TAGACACGTCCAAGAACCAGTTCTCCCTGAA |
| | ACTGAGCTCTGTTACCGCCGCAGACACGGCT |
| | GTCTATTACTGTGCGAGATCACGTTGGGGCA |
| | GCAGCTGGGTATTTGACTACTGGGGCCAGGG |
| | CACACTGGTCACCGTCTCTTCG |
| NI-101.12F | CAGGTGCAGCTGGTGGAGTCTGGGGGAGGCG |
| 6A | TGGTCCAGCCTGGGAGGTCCCTGAGACTCTC |
| (SEQ ID NO: 38) | CTGTGCAGCGTCTGGATTCGCCTTCAGTAGC |
| | TATGGCATGCACTGGGTCCGCCAGGCTCCAG |
| | GCAAGGGGCTGGAGTGGGTGGCAGTTATATG |
| | GTTTGATGGAACTAAAAAATACTATACAGAC |
| | TCCGTGAAGGGCAGATTCACCATCTCCAGAG |
| | ACAATTCCAAGAACACACTGTATCTGCAAAT |
| | GAACACCCTGAGAGCCGAGGACACGGCTGTG |
| | TATTACTGTGCGAGAGATAGGGGTATAGGAG |
| | CTCGGCGGGGGCCGTACTACATGGACGTCTG |
| | GGGCAAAGGGACCACGGTCACCGTCTCCTCA |
| NI-101.13A | CAGGTGCAGCTGCAGGAGTCGGGCCCAGGAC |
| (SEQ ID NO: 52) | TGGTGAAGCCTTCGGAGACCCTGTCCCTCAC |
| | CTGCACTGTCTCTGGTGGCTCCATCAGCAGA |
| | AGAAGTTACTACTGGGGCTGGATCCGCCAGT |
| | CCCCAGGGAAGGGGCTGGAGTGGAGTGGAAG |
| | TATCCATTATAGCGGGAGCACCTACTACAAC |
| | CCGTCCCTCAAGAGTCGAGTCACCATATCTG |
| | TAGACACGTCCAAGAACCAGTTCTCCCTGAA |
| | ACTGAGCTCTGTTACCGCCGCAGACACGGCT |
| | GTCTATTACTGTGCGAGATCACGTTGGGGCA |
| | GCAGCTGGGTATTTGACTACTGGGGCCAGGG |
| | AACCCTGGTCACCGTCTCCTCG |
| NI-101.13B | CAGGTGCAGCTGCAGGAGTCGGGCCCAGGAC |
| (SEQ ID NO: 53) | TGGTGAAGCCTTCGGAGACCCTGTCCCTCAC |
| | CTGCACTGTCTCTGGTGGCTCCATCAGCAGA |
| | AGAAGTTACTACTGGGGCTGGATCCGCCAGT |
| | CCCCAGGGAAGGGGCTGGAGTGGAGTGGAAG |
| | TATCCATTATAGCGGGAGCACCTACTACAAC |
| | CCGTCCCTCAAGAGTCGAGTCACCATATCTG |
| | TAGACACGTCCAAGAACCAGTTCTCCCTGAA |
| | ACTGAGCTCTGTTACCGCCGCAGACACGGCT |
| | GTCTATTACTGTGCGAGATCACGTTGGGGCA |
| | GCAGCTGGGTATTTGACTACTGGGGCCAGGG |
| | AACCCTGGTCACCGTCTCCTCG |

TABLE 6
Polynucleotide sequences of the V_{L} region.

| | Variable light chain sequence |
|---|---|
| Antibody | (kappa or lambda) |
| NI-101.10 | GAAATTGTGCTGACTCAGTCTCCATCCTCCCT |
| and | GTCTGCATCTGTAGGAGACAGAGTCACCATCA |
| NI-101.11 | CTTGCCGGGCAAGTCAGAGCATTAGCAGCTAT |
| (SEQ ID NO: 7) | TTAAATTGGTATCAACAGAAACCAGGGAAAGC |
| | CCCTAAGCTCCTGATCTATGCTGCATCCAGTT |
| | TGCAAAGTGGGGTCCCATCAAGGTTCAGTGGC |
| | AGTGGATCTGGGACAGATTTCACTCTCACCAT |
| | CAGCAGTCTGCAACCTGAAGATTTTGCAACTT |
| | ATTACTGTCAGCAGAGTTACAGTACCCCTCTC |
| | ACTTTCGGCGGAGGGACCAAGCTCGAGATCAA |
| | ACGTACG |
| NI-101.12 | GACGAGATCGTGCTGACCCAGAGCCCCAGCAG |
| (SEQ ID NO: 11) | CCTGAGCGCCAGCATCGGCGACCGGGTGACCA |
| | TCACCTGCCGGGCCAGCGAGAGCATCAACAAG |
| | TACGTGAACTGGTACCAGCAGAAGCCCGGCAA |
| | GGCCCCCAAGCTGCTGATCTACGCCGCCAGCA |
| | GCCTGCAGAGCGGCGCCCCCAGCCGGGTGAGC |
| | GGCAGCGGCTTCGGCCGGGACTTCAGCCTGAC |
| | CATCAGCGGCCTGCAGGCCGAGGACTTCGGCG |
| | CCTACTTCTGCCAGCAGAGCTACAGCGCCCCC |
| | TACACCTTCGGCCAGGGCACCAAGGTGGAGAT |
| | CAAGCGGACC |
| NI-101.13 | CAGAGCGTGCTGACCCAGCCGCCGAGCGCGAG |
| (SEQ ID NO: 15) | CGGCACCCCGGGCCAGCGCGTGACCATTAGCT |
| | GCAGCGGCAGCAGCAGCAACATTGGCAGCAAC |
| | TATGTGTATTGGTATCAGCAGCCGCCGGGCAC |
| | CGCGCCGAAACTGCTGATTTATCGCAACAACC |
| | AGCGCCCGAGCGGCGTGCCGGATCGCTTTAGC |
| | GGCAGCAAAAGCGGCACCAGCGCGAGCCTGGC |
| | GATTAGCGGCCTGCGCAGCGAAGATGAAGCGG |
| | ATTATTATTGCGCGGCGTGGGATGATAGCCTG |
| | AGCGGCTATGTGTTTGGCACCGGCACCAAAGT |
| | GACCGTGCTG |
| NI-101.12F | GACATCCAGATGACCCAGTCTCCATCCTCCCT |
| 6A | GTCTGCATCTGTAGGAGACAGAGTCACCATCA |
| (SEQ ID NO: 40) | CTTGCCGGGCAAGTCAGAGCATTAGCAGCTAT |
| | TTAAATTGGTATCAACAGAAACCAGGGAAAGC |
| | CCCTAAGCTCCTGATCTATGCTGCATCCAGTT |
| | TGCAAAGTGGGGTCCCATCAAGGTTCAGTGGC |
| | AGTGGATCTGGGACAGATTTCACTCTCACCAT |
| | CAGCAGTCTGCAACCTGAAGATTTTGCAACTT |
| | ATTACTGTCAGCAGAGTTACAGTACCCCTCTC |
| | ACTTTCGGCGGAGGGACCAAGGTGGAGATCAA |
| | ACGT |
| NI-101.13A | GACATCCAGTTGACCCAGTCTCCATCCTCCCT |
| (SEQ ID NO: 54) | GTCTGCATCTGTAGGAGACAGAGTCACCATCA |
| | CTTGCCGGGCAAGTCAGAGCATTAGCAGCTAT |
| | TTAAATTGGTATCAGCAGAAACCAGGGAAAGC |
| | CCCTAAGCTCCTGATCTATGCTGCATCCAGTT |
| | AACAAAGTGGGGTCCCATCAAGGTTCAGTGGC |
| | AGTGGATCTGGGACAGATTTCACTCTCACCAT |
| | CAGCAGTCTGCAACCTGAAGATTTTGCAACTT |
| | ACTACTGTCAACAGAGTTACAGTACCAGAACG |
| | TTCGGCCAAGGGACCAAGGTGGAGATCAAACG |
| | TACG |
| NI-101.13B | GACATCCAGTTGACCCAGTCTCCTTCCACCCT |
| (SEQ ID NO: 2) | GTCTGCATCTGTAGGAGACAGAGTCACCATCA |
| | CTTGCCGGGCCAGTCAGAGTATTAGTAGCTGG |
| | TTGGCCTGGTATCAGCAGATTCCAGGGAAAGC |
| | CCCTAAGCTCCTGATCTATAAGGCGTCTAGTT |
| | TAGAAAGTGGGGTCCCATCAAGGTTCAGCGGC |
| | AGTGGATCTGGGACAGAATTCACTCTCACCAT |
| | CAGCAGCCTGCAGCCTGATGATTTTGCAACTT |
| | ATTACTGCCAACAGTATAATAGTTATTCTCGA |
| | ACGTTCGGCCAAGGGACCAAGCTGGAGATCAA |
| | ACGTACG |

In this respect, one of skill in the art will readily appreciate that the polynucleotides encoding at least the variable domain of the light and/or heavy chain can encode the variable domains of both immunoglobulin chains or only one. Likewise, said polynucleotides can be under the control of the same promoter or can be separately controlled for expression. Possible regulatory elements permitting expression in prokaryotic host cells comprise, e.g., the P_{L}, lac, trp or tac promoter in E. coli, and examples for regulatory elements permitting expression in eukaryotic host cells are the AOX1 or GAL1 promoter in yeast or the CMV-, SV40-, RSV-promoter, CMV-enhancer, SV40-enhancer or a globin intron in mammalian and other animal cells. Beside elements that are responsible for the initiation of transcription such regulatory elements can also comprise transcription termination signals, such as the SV40-poly-A site or the tk-poly-A site, downstream of the polynucleotide. Furthermore, depending on the expression system used leader sequences capable of directing the polypeptide to a cellular compartment or secreting it into the medium can be added to the coding sequence of the polynucleotide and are well known in the art. The leader sequence(s) is (are) assembled in appropriate phase with translation, initiation and termination sequences, and in some embodiments, a leader sequence capable of directing secretion of translated protein, or a portion thereof, into the periplasmic space or extracellular medium. Optionally, the heterologous sequence can encode a fusion protein including a C- or N-terminal identification peptide imparting desired characteristics, e.g., stabilization or simplified purification of expressed recombinant product. In this context, suitable expression vectors are known in the art such as Okayama-Berg cDNA expression vector pcDV1 (Pharmacia), pCDM8, pRc/CMV, pcDNA1, pcDNA3 (Invitrogen), or pSPORT1 (GIBCO BRL). The expression control sequences can be eukaryotic promoter systems in vectors capable of transforming or transfecting eukaryotic host cells, but control sequences for prokaryotic hosts can also be used. Once the vector has been incorporated into the appropriate host, the host is maintained under conditions suitable for high level expression of the nucleotide sequences, and, as desired, the collection and purification of the immunoglobulin light chains, heavy chains, light/heavy chain dimers or intact antibodies, binding fragments or other immunoglobulin forms can follow; see, Beychok, Cells of Immunoglobulin Synthesis, Academic Press, N.Y., (1979).

The present methods also include use of fragments of the polynucleotides, as described elsewhere. Additionally polynucleotides that encode fusion polynucleotides, Fab fragments, and other derivatives, as described herein, are also contemplated for use.

The polynucleotides can be produced or manufactured by any method known in the art. For example, if the nucleotide sequence of the antibody is known, a polynucleotide encoding the antibody can be assembled from chemically synthesized oligonucleotides (e.g., as described in Kutmeier et al., BioTechniques 17:242 (1994)), which, briefly, involves the synthesis of overlapping oligonucleotides containing portions of the sequence encoding the antibody, annealing and ligating of those oligonucleotides, and then amplification of the ligated oligonucleotides by PCR.

Alternatively, a polynucleotide encoding an antibody, or antigen-binding fragment, variant, or derivative thereof can be generated from nucleic acid from a suitable source. If a clone containing a nucleic acid encoding a particular antibody is not available, but the sequence of the antibody molecule is known, a nucleic acid encoding the antibody can be chemically synthesized or obtained from a suitable source (e.g., an antibody cDNA library, or a cDNA library generated from nucleic acid, such as poly A+RNA, isolated from, any tissue or cells expressing the neoantigen-specific antibody, such as hybridoma cells selected to express an antibody) by PCR amplification using synthetic primers hybridizable to the 3′ and 5′ ends of the sequence or by cloning using an oligonucleotide probe specific for the particular gene sequence to identify, e.g., a cDNA clone from a cDNA library that encodes the antibody. Amplified nucleic acids generated by PCR can then be cloned into replicable cloning vectors using any method well known in the art.

Once the nucleotide sequence and corresponding amino acid sequence of the antibody, or antigen-binding fragment, variant, or derivative thereof is determined, its nucleotide sequence can be manipulated using methods well known in the art for the manipulation of nucleotide sequences, e.g., recombinant DNA techniques, site directed mutagenesis, PCR, etc. (see, for example, the techniques described in Sambrook et al., Molecular Cloning, A Laboratory Manual, 2d Ed., Cold Spring Harbor Laboratory, Cold Spring Harbor, N.Y. (1990) and Ausubel et al., eds., Current Protocols in Molecular Biology, John Wiley & Sons, NY (1998), which are both incorporated by reference herein in their entireties), to generate antibodies having a different amino acid sequence, for example to create amino acid substitutions, deletions, and/or insertions.

V. Expression of Antibody Polypeptides

Means and methods for the recombinant production of Abeta binding molecules, in particular antibodies and mimics thereof as well as methods of screening for competing Abeta binding molecules, which may or may not be antibodies, are known in the art and are summarized, for example, in international application WO2006/103116 with respect to antibodies against beta-amyloid and the treatment/diagnosis of Alzheimer's disease, the disclosure of which is incorporated herein by reference for this purpose of antibody engineering and administration for therapeutic or diagnostic applications.

The methods also include a method for producing cells capable of expressing an antibody or its corresponding immunoglobulin chain(s) comprising genetically engineering cells with the polynucleotide or with the vector as described herein. The cells obtainable by the methods described herein can be used, for example, to test the interaction of the antibody with its antigen.

Following manipulation of the isolated genetic material to provide antibodies, or antigen-binding fragments, variants, or derivatives thereof, the polynucleotides encoding the antibodies are typically inserted in an expression vector for introduction into host cells that can be used to produce the desired quantity of antibody.

Recombinant expression of an antibody, or fragment, derivative or analog thereof, e.g., a heavy or light chain of an antibody that binds to a target molecule described herein. Once a polynucleotide encoding an antibody molecule or a heavy or light chain of an antibody, or portion thereof (or containing the heavy or light chain variable domain), has been obtained, the vector for the production of the antibody molecule can be produced by recombinant DNA technology using techniques well known in the art. Thus, methods for preparing a protein by expressing a polynucleotide containing an antibody encoding nucleotide sequence are described herein. Methods that are well known to one of skill in the art can be used to construct expression vectors containing antibody coding sequences and appropriate transcriptional and translational control signals. These methods include, for example, in vitro recombinant DNA techniques, synthetic techniques, and in vivo genetic recombination. The description herein, thus, provides replicable vectors comprising a nucleotide sequence encoding an antibody molecule, or a heavy or light chain thereof, or a heavy or light chain variable domain, operably linked to a promoter. Such vectors can include the nucleotide sequence encoding the constant region of the antibody molecule (see, e.g., PCT Publication WO 86/05807; PCT Publication WO 89/01036; and U.S. Pat. No. 5,122,464) and the variable domain of the antibody can be cloned into such a vector for expression of the entire heavy or light chain.

The present description relates to vectors, particularly plasmids, cosmids, viruses and bacteriophages used conventionally in genetic engineering that comprise a polynucleotide encoding the antigen or a variable domain of an immunoglobulin chain of an antibody; optionally in combination with a polynucleotide that encodes the variable domain of the other immunoglobulin chain of the antibody of the invention. Said vector can be an expression vector and/or a gene transfer or targeting vector. Expression vectors derived from viruses such as retroviruses, vaccinia virus, adeno-associated virus, herpes viruses, or bovine papilloma virus, can be used for delivery of the polynucleotides or vector into targeted cell population. Methods that are well known to one of skill in the art can be used to construct recombinant viral vectors; see, for example, the techniques described in Sambrook, Molecular Cloning A Laboratory Manual, Cold Spring Harbor Laboratory (1989) N.Y. and Ausubel, Current Protocols in Molecular Biology, Green Publishing Associates and Wiley Interscience, N.Y. (1994). Alternatively, the polynucleotides and vectors can be reconstituted into liposomes for delivery to target cells. The vectors containing the polynucleotides (e.g., the heavy and/or light variable domain(s) of the immunoglobulin chains encoding sequences and expression control sequences) can be transferred into the host cell by well known methods, which vary depending on the type of cellular host. For example, calcium chloride transfection is commonly utilized for prokaryotic cells, whereas calcium phosphate treatment or electroporation can be used for other cellular hosts; see Sambrook, supra.

The term “vector” or “expression vector” is used herein to mean vectors used in accordance with the present methods as a vehicle for introducing into and expressing a desired gene in a host cell. As known to one of skill in the art, such vectors can easily be selected from the group consisting of plasmids, phages, viruses and retroviruses. In general, vectors compatible with the instant methods will comprise a selection marker, appropriate restriction sites to facilitate cloning of the desired gene and the ability to enter and/or replicate in eukaryotic or prokaryotic cells.

For the purposes of the methods described herein, numerous expression vector systems can be employed. For example, one class of vector utilizes DNA elements that are derived from animal viruses such as bovine papilloma virus, polyoma virus, adenovirus, vaccinia virus, baculovirus, retroviruses (RSV, MMTV or MOMLV) or SV40 virus. Others involve the use of polycistronic systems with internal ribosome binding sites. Additionally, cells that have integrated the DNA into their chromosomes can be selected by introducing one or more markers that allow selection of transfected host cells. The marker can provide for prototrophy to an auxotrophic host, biocide resistance (e.g., antibiotics) or resistance to heavy metals such as copper. The selectable marker gene can either be directly linked to the DNA sequences to be expressed, or introduced into the same cell by cotransformation. Additional elements can also be needed for optimal synthesis of mRNA. These elements can include signal sequences, splice signals, as well as transcriptional promoters, enhancers, and termination signals.

In particular embodiments, the cloned variable region genes are inserted into an expression vector along with the heavy and light chain constant region genes (such as human) synthetic as discussed above. In one embodiment, this is effected using a proprietary expression vector of Biogen DEC, Inc., referred to as NEOSPLA (disclosed in U.S. Pat. No. 6,159,730). This vector contains the cytomegalovirus promoter/enhancer, the mouse beta globin major promoter, the SV40 origin of replication, the bovine growth hormone polyadenylation sequence, neomycin phosphotransferase exon 1 and exon 2, the dihydrofolate reductase gene and leader sequence. This vector has been found to result in very high level expression of antibodies upon incorporation of variable and constant region genes, transfection in CHO cells, followed by selection in G418 containing medium and methotrexate amplification. Of course, any expression vector that is capable of eliciting expression in eukaryotic cells can be used in the present methods. Examples of suitable vectors include, but are not limited to plasmids pcDNA3, pHCMV/Zeo, pCR3.1, pEF1/His, pIND/GS, pRc/HCMV2, pSV40/Zeo2, pTRACER-HCMV, pUB6/V5-His, pVAX1, and pZeoSV2 (available from Invitrogen, San Diego, Calif.), and plasmid pCI (available from Promega, Madison, Wis.). In general, screening large numbers of transformed cells for those that express suitably high levels if immunoglobulin heavy and light chains is routine experimentation that can be carried out, for example, by robotic systems. Vector systems are also taught in U.S. Pat. Nos. 5,736,137 and 5,658,570, each of which is incorporated by reference in its entirety herein. This system provides for high expression levels, e.g., >30 pg/cell/day. Other exemplary vector systems are disclosed e.g., in U.S. Pat. No. 6,413,777.

In other embodiments the antibodies, or antigen-binding fragments, variants, or derivatives thereof for use in the methods described herein can be expressed using polycistronic constructs such as those disclosed in United States Patent Application Publication No. 2003-0157641 A1, filed Nov. 18, 2002 and incorporated herein in its entirety. In these novel expression systems, multiple gene products of interest such as heavy and light chains of antibodies can be produced from a single polycistronic construct. These systems advantageously use an internal ribosome entry site (IRES) to provide relatively high levels of antibodies. Compatible IRES sequences are disclosed in U.S. Pat. No. 6,193,980 which is also incorporated herein. One of skill in the art will appreciate that such expression systems can be used to effectively produce the full range of antibodies disclosed in the instant application.

More generally, once the vector or DNA sequence encoding a monomeric subunit of the antibody has been prepared, the expression vector can be introduced into an appropriate host cell. Introduction of the plasmid into the host cell can be accomplished by various techniques well known to one of skill in the art. These include, but are not limited to, transfection (including electrophoresis and electroporation), protoplast fusion, calcium phosphate precipitation, cell fusion with enveloped DNA, microinjection, and infection with intact virus. See, Ridgway, A. A. G. “Mammalian Expression Vectors” Vectors, Rodriguez and Denhardt, Eds., Butterworths, Boston, Mass., Chapter 24.2, pp. 470-472 (1988). Typically, plasmid introduction into the host is via electroporation. The host cells harboring the expression construct are grown under conditions appropriate to the production of the light chains and heavy chains, and assayed for heavy and/or light chain protein synthesis. Exemplary assay techniques include enzyme-linked immunosorbent assay (ELISA), radioimmunoassay (RIA), or fluorescence-activated cell sorter analysis (FACS), immunohistochemistry and the like.

The expression vector is transferred to a host cell by conventional techniques and the transfected cells are then cultured by conventional techniques to produce an antibody for use in the methods described herein. Thus, the methods provided herein include the use of host cells containing a polynucleotide encoding an antibody, or a heavy or light chain thereof, operably linked to a heterologous promoter. In embodiments for the expression of double-chained antibodies, vectors encoding both the heavy and light chains can be co-expressed in the host cell for expression of the entire immunoglobulin molecule, as detailed below.

The present description furthermore relates to host cells transformed with a polynucleotide or vector described herein. Said host cell can be a prokaryotic or eukaryotic cell. The polynucleotide or vector that is present in the host cell can either be integrated into the genome of the host cell or it can be maintained extrachromosomally. The host cell can be any prokaryotic or eukaryotic cell, such as a bacterial, insect, fungal, plant, animal or human cell. Fungal cells are, for example, those of the genus Saccharomyces, in particular those of the species S. cerevisiae. The term “prokaryotic” is meant to include all bacteria that can be transformed or transfected with a DNA or RNA molecules for the expression of an antibody or the corresponding immunoglobulin chains. Prokaryotic hosts can include gram negative as well as gram positive bacteria such as, for example, E. coli, S. typhimurium, Serratia marcescens and Bacillus subtilis. The term “eukaryotic” is meant to include yeast, higher plant, insect and mammalian cells, HEK 293, NSO and CHO cells. Depending upon the host employed in a recombinant production procedure, the antibodies or immunoglobulin chains encoded by the polynucleotide can be glycosylated or can be non-glycosylated. Antibodies or the corresponding immunoglobulin chains can also include an initial methionine amino acid residue. A polynucleotide can be used to transform or transfect the host using any of the techniques commonly known to one of skill in the art. Furthermore, methods for preparing fused, operably linked genes and expressing them in, e.g., mammalian cells and bacteria are well-known in the art (Sambrook, Molecular Cloning: A Laboratory Manual, Cold Spring Harbor Laboratory, Cold Spring Harbor, N.Y., 1989). The genetic constructs and methods described therein can be utilized for expression of the antibody or the corresponding immunoglobulin chains in eukaryotic or prokaryotic hosts. In general, expression vectors containing promoter sequences that facilitate the efficient transcription of the inserted polynucleotide are used in connection with the host. The expression vector typically contains an origin of replication, a promoter, and a terminator, as well as specific genes that are capable of providing phenotypic selection of the transformed cells. Suitable source cells for the DNA sequences and host cells for immunoglobulin expression and secretion can be obtained from a number of sources, such as the American Type Culture Collection (“Catalogue of Cell Lines and Hybridomas,” Eighth edition (1994) Rockville, Md., U.S.A., which is incorporated herein by reference). Furthermore, transgenic animals, such as mammals, comprising cells can be used for the large scale production of the antibody.

The transformed hosts can be grown in fermentors and cultured according to techniques known in the art to achieve optimal cell growth. Once expressed, the whole antibodies, their dimers, individual light and heavy chains, or other immunoglobulin forms, can be purified according to standard procedures of the art, including ammonium sulfate precipitation, affinity columns, column chromatography, gel electrophoresis and the like; see, Scopes, “Protein Purification”, Springer Verlag, N.Y. (1982). The antibody or its corresponding immunoglobulin chain(s) can then be isolated from the growth medium, cellular lysates, or cellular membrane fractions. The isolation and purification of the, e.g., recombinantly expressed antibodies or immunoglobulin chains can be by any conventional means such as, for example, preparative chromatographic separations and immunological separations such as those involving the use of monoclonal or polyclonal antibodies directed, e.g., against the constant region of the antibody. It will be apparent to one of skill in the art that the antibodies can be further coupled to other moieties for, e.g., drug targeting and imaging applications. Such coupling can be conducted chemically after expression of the antibody or antigen to site of attachment or the coupling product can be engineered into the antibody or antigen at the DNA level. The DNAs are then expressed in a suitable host system, and the expressed proteins are collected and renatured, if necessary.

Substantially pure immunoglobulins of at least about 90 to 95% homogeneity or at least about 98 to 99% or more homogeneity can be used for pharmaceutical uses. Once purified, partially or to homogeneity as desired, the antibodies can then be used therapeutically (including extracorporally) or in developing and performing assay procedures.

The host cell can be co-transfected with two expression vectors, the first vector encoding a heavy chain derived polypeptide and the second vector encoding a light chain derived polypeptide. The two vectors can contain identical selectable markers that enable equal expression of heavy and light chain polypeptides. Alternatively, a single vector can be used that encodes both heavy and light chain polypeptides. In such situations, the light chain is advantageously placed before the heavy chain to avoid an excess of toxic free heavy chain (Proudfoot, Nature 322:52 (1986); Kohler, Proc. Natl. Acad. Sci. USA 77:2197 (1980)). The coding sequences for the heavy and light chains can comprise cDNA or genomic DNA.

As used herein, “host cells” refers to cells that harbor vectors constructed using recombinant DNA techniques and encoding at least one heterologous gene. In descriptions of processes for isolation of antibodies from recombinant hosts, the terms “cell” and “cell culture” are used interchangeably to denote the source of antibody unless it is clearly specified otherwise. In other words, recovery of polypeptide from the “cells” can mean either from spun down whole cells, or from the cell culture containing both the medium and the suspended cells.

A variety of host-expression vector systems can be utilized to express antibody molecules for use in the methods described herein. Such host-expression systems represent vehicles by which the coding sequences of interest can be produced and subsequently purified, but also represent cells that can, when transformed or transfected with the appropriate nucleotide coding sequences, express an antibody molecule in situ. These include but are not limited to microorganisms such as bacteria (e.g., E. coli, B. subtilis) transformed with recombinant bacteriophage DNA, plasmid DNA or cosmid DNA expression vectors containing antibody coding sequences; yeast (e.g., Saccharomyces, Pichia) transformed with recombinant yeast expression vectors containing antibody coding sequences; insect cell systems infected with recombinant virus expression vectors (e.g., baculovirus) containing antibody coding sequences; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing antibody coding sequences; or mammalian cell systems (e.g., COS, CHO, BLK, 293, 3T3 cells) harboring recombinant expression constructs containing promoters derived from the genome of mammalian cells (e.g., metallothionein promoter) or from mammalian viruses (e.g., the adenovirus late promoter; the vaccinia virus 7.5K promoter). Bacterial cells such as Escherichia coli, and eukaryotic cells, especially for the expression of whole recombinant antibody molecule, are used for the expression of a recombinant antibody molecule. For example, mammalian cells such as Chinese hamster ovary cells (CHO), in conjunction with a vector such as the major intermediate early gene promoter element from human cytomegalovirus is an effective expression system for antibodies (Foecking et al., Gene 45:101 (1986); Cockett et al., Bio/Technology 8:2 (1990)).

The host cell line used for protein expression can be of mammalian origin; one of skill in the art is credited with ability to determine particular host cell lines that are best suited for the desired gene product to be expressed therein. Exemplary host cell lines include, but are not limited to, CHO (Chinese Hamster Ovary), DG44 and DUXB11 (Chinese Hamster Ovary lines, DHFR minus), HELA (human cervical carcinoma), CV1 (monkey kidney line), COS (a derivative of CV1 with SV40 T antigen), VERY, BHK (baby hamster kidney), MDCK, 293, WI38, R1610 (Chinese hamster fibroblast) BALBC/3T3 (mouse fibroblast), HAK (hamster kidney line), SP2/O (mouse myeloma), P3×63-Ag3.653 (mouse myeloma), BFA-1c1BPT (bovine endothelial cells), RAJI (human lymphocyte) and 293 (human kidney). Host cell lines are typically available from commercial services, the American Tissue Culture Collection or from published literature.

In addition, a host cell strain can be chosen that modulates the expression of the inserted sequences, or modifies and processes the gene product in the specific fashion desired. Such modifications (e.g., glycosylation) and processing (e.g., cleavage) of protein products can be important for the function of the protein. Different host cells have characteristic and specific mechanisms for the post-translational processing and modification of proteins and gene products. Appropriate cell lines or host systems can be chosen to ensure the correct modification and processing of the foreign protein expressed. To this end, eukaryotic host cells that possess the cellular machinery for proper processing of the primary transcript, glycosylation, and phosphorylation of the gene product can be used.

For long-term, high-yield production of recombinant proteins, stable expression can be used. For example, cell lines that stably express the antibody molecule can be engineered. Rather than using expression vectors that contain viral origins of replication, host cells can be transformed with DNA controlled by appropriate expression control elements (e.g., promoter, enhancer, sequences, transcription terminators, polyadenylation sites, etc.), and a selectable marker. Following the introduction of the foreign DNA, engineered cells can be allowed to grow for 1-2 days in an enriched media, and then are switched to a selective media. The selectable marker in the recombinant plasmid confers resistance to the selection and allows cells to stably integrate the plasmid into their chromosomes and grow to form foci that in turn can be cloned and expanded into cell lines. This method can advantageously be used to engineer cell lines that stably express the antibody molecule.

A number of selection systems can be used, including but not limited to the herpes simplex virus thymidine kinase (Wigler et al., Cell 11:223 (1977)), hypoxanthine-guanine phosphoribosyltransferase (Szybalska & Szybalski, Proc. Natl. Acad. Sci. USA 48:202 (1992)), and adenine phosphoribosyltransferase (Lowy et al., Cell 22:817 1980) genes can be employed in tk-, hgprt- or aprt-cells, respectively. Also, anti-metabolite resistance can be used as the basis of selection for the following genes: dhfr, which confers resistance to methotrexate (Wigler et al., Natl. Acad. Sci. USA 77:357 (1980); O'Hare et al., Proc. Natl. Acad. Sci. USA 78:1527 (1981)); gpt, which confers resistance to mycophenolic acid (Mulligan & Berg, Proc. Natl. Acad. Sci. USA 78:2072 (1981)); neo, which confers resistance to the aminoglycoside G-418 Clinical Pharmacy 12:488-505; Wu and Wu, Biotherapy 3:87-95 (1991); Tolstoshev, Ann. Rev. Pharmacol. Toxicol. 32:573-596 (1993); Mulligan, Science 260:926-932 (1993); and Morgan and Anderson, Ann. Rev. Biochem. 62:191-217 (1993); TIB TECH 11(5):155-215 (May, 1993); and hygro, which confers resistance to hygromycin (Santerre et al., Gene 30:147 (1984). Methods commonly known in the art of recombinant DNA technology that can be used are described in Ausubel et al. (eds.), Current Protocols in Molecular Biology, John Wiley & Sons, NY (1993); Kriegler, Gene Transfer and Expression, A Laboratory Manual, Stockton Press, NY (1990); and in Chapters 12 and 13, Dracopoli et al. (eds), Current Protocols in Human Genetics, John Wiley & Sons, NY (1994); Colberre-Garapin et al., J. Mol. Biol. 150:1 (1981), which are incorporated by reference herein in their entireties.

The expression levels of an antibody molecule can be increased by vector amplification (for a review, see Bebbington and Hentschel, The use of vectors based on gene amplification for the expression of cloned genes in mammalian cells in DNA cloning, Academic Press, New York, Vol. 3. (1987)). When a marker in the vector system expressing antibody is amplifiable, increase in the level of inhibitor present in culture of host cell will increase the number of copies of the marker gene. Since the amplified region is associated with the antibody gene, production of the antibody will also increase (Crouse et al., Mol. Cell. Biol. 3:257 (1983)).

In vitro production allows scale-up to give large amounts of the desired polypeptides. Techniques for mammalian cell cultivation under tissue culture conditions are known in the art and include homogeneous suspension culture, e.g. in an airlift reactor or in a continuous stirrer reactor, or immobilized or entrapped cell culture, e.g. in hollow fibers, microcapsules, on agarose microbeads or ceramic cartridges. If necessary and/or desired, the solutions of polypeptides can be purified by the customary chromatography methods, for example gel filtration, ion-exchange chromatography, chromatography over DEAE-cellulose or (immuno-)affinity chromatography, e.g., after biosynthesis of a synthetic hinge region polypeptide or prior to or subsequent to the HIC chromatography step described herein.

Genes encoding antibodies, or antigen-binding fragments, variants, or derivatives thereof for use in the methods described herein can also be expressed non-mammalian cells such as bacteria or insect or yeast or plant cells. Bacteria that readily take up nucleic acids include members of the enterobacteriaceae, such as strains of Escherichia coli or Salmonella; Bacillaceae, such as Bacillus subtilis; Pneumococcus; Streptococcus, and Haemophilus influenzae. It will further be appreciated that, when expressed in bacteria, the heterologous polypeptides typically become part of inclusion bodies. The heterologous polypeptides must be isolated, purified and then assembled into functional molecules. Where tetravalent forms of antibodies are desired, the subunits will then self-assemble into tetravalent antibodies (WO02/096948A2).

In bacterial systems, a number of expression vectors can be advantageously selected depending upon the use intended for the antibody molecule being expressed. For example, when a large quantity of such a protein is to be produced, for the generation of pharmaceutical compositions of an antibody molecule, vectors that direct the expression of high levels of fusion protein products that are readily purified can be desirable. Such vectors include, but are not limited, to the E. coli expression vector pUR278 (Ruther et al., EMBO J. 2:1791 (1983)), in which the antibody coding sequence can be ligated individually into the vector in frame with the lacZ coding region so that a fusion protein is produced; pIN vectors (Inouye & Inouye, Nucleic Acids Res. 13:3101-3109 (1985); Van Heeke & Schuster, J. Biol. Chem. 24:5503-5509 (1989)); and the like. pGEX vectors can also be used to express foreign polypeptides as fusion proteins with glutathione S-transferase (GST). In general, such fusion proteins are soluble and can easily be purified from lysed cells by adsorption and binding to a matrix glutathione-agarose beads followed by elution in the presence of free glutathione. The pGEX vectors are designed to include thrombin or factor Xa protease cleavage sites so that the cloned target gene product can be released from the GST moiety.

In addition to prokaryotes, eukaryotic microbes can also be used. Saccharomyces cerevisiae, or common baker's yeast, is the most commonly used among eukaryotic microorganisms although a number of other strains are commonly available, e.g., Pichia pastoris.

For expression in Saccharomyces, the plasmid YRp7, for example, (Stinchcomb et al., Nature 282:39 (1979); Kingsman et al., Gene 7:141 (1979); Tschemper et al., Gene 10:157 (1980)) is commonly used. This plasmid already contains the TRP1 gene, which provides a selection marker for a mutant strain of yeast lacking the ability to grow in tryptophan, for example ATCC No. 44076 or PEP4-1 (Jones, Genetics 85:12 (1977)). The presence of the trp1 lesion as a characteristic of the yeast host cell genome then provides an effective environment for detecting transformation by growth in the absence of tryptophan.

In an insect system, Autographa californica nuclear polyhedrosis virus (AcNPV) is typically used as a vector to express foreign genes. The virus grows in Spodoptera frugiperda cells. The antibody coding sequence can be cloned individually into non-essential regions (for example the polyhedrin gene) of the virus and placed under control of an AcNPV promoter (for example the polyhedrin promoter).

Once an antibody molecule has been recombinantly expressed, it can be purified by any method known in the art for purification of an immunoglobulin molecule, for example, by chromatography (e.g., ion exchange, affinity, particularly by affinity for the specific antigen after Protein A, and sizing column chromatography), centrifugation, differential solubility, or by any other standard technique for the purification of proteins. Alternatively, another method for increasing the affinity of antibodies is disclosed in US 2002/0123057 A1.

VI. Fusion Proteins and Conjugates

The antibodies for use in the methods described herein can comprise a further domain, said domain being linked by covalent or non-covalent bonds. The linkage can be based on genetic fusion according to the methods known in the art and described above or can be performed by, e.g., chemical cross-linking as described in, e.g., international application WO94/04686. The additional domain present in the fusion protein comprising the antibody can be linked by a flexible linker, advantageously a polypeptide linker, wherein said polypeptide linker comprises plural, hydrophilic, peptide-bonded amino acids of a length sufficient to span the distance between the C-terminal end of said further domain and the N-terminal end of the antibody or vice versa. The therapeutically or diagnostically active agent can be coupled to the antibody or an antigen-binding fragment thereof by various means. This includes, for example, single-chain fusion proteins comprising the variable regions of the antibody coupled by covalent methods, such as peptide linkages, to the therapeutically or diagnostically active agent. Further examples include molecules that comprise at least an antigen-binding fragment coupled to additional molecules covalently or non-covalently include those in the following non-limiting illustrative list. Traunecker, Int. J. Cancer Surp. SuDP 7 (1992), 51-52, describe the bispecific reagent janusin in which the Fv region directed to CD3 is coupled to soluble CD4 or to other ligands such as OVCA and IL-7. Similarly, the variable regions of the antibody can be constructed into Fv molecules and coupled to alternative ligands such as those illustrated in the cited article. Higgins, J. Infect. Disease 166 (1992), 198-202, described a hetero-conjugate antibody composed of OKT3 cross-linked to an antibody directed to a specific sequence in the V3 region of GP120. Such hetero-conjugate antibodies can also be constructed using at least the variable regions contained in the antibody methods. Additional examples of specific antibodies include those described by Fanger, Cancer Treat. Res. 68 (1993), 181-194 and by Fanger, Crit. Rev. Immunol. 12 (1992), 101-124.

In a further embodiment, the Abeta binding molecule, antibody, immunoglobulin chain or a binding fragment thereof or the antigen is detectably labeled. Labeling agents can be coupled either directly or indirectly to the antibodies or antigens. One example of indirect coupling is by use of a spacer moiety.

Hence, the biological activity of the Abeta binding molecules, e.g. antibodies identified herein indicates that they have sufficient affinity to make them candidates for drug localization to cells expressing the appropriate surface structures of the diseased cell and tissue, respectively. This targeting and binding to cells could be useful for the delivery of therapeutically or diagnostically active agents and gene therapy/gene delivery. Molecules/particles with an antibody would bind specifically to cells/tissues expressing the variant form of the pathological protein, and therefore could have diagnostic and therapeutic use. Thus, the Abeta binding molecule, e.g., antibody or antigen binding fragment thereof for use in the methods described herein can be labeled (e.g., fluorescent, radioactive, enzyme, nuclear magnetic, heavy metal) and used to detect specific targets in vivo or in vitro including “immunochemistry” like assays in vitro. In vivo they could be used in a manner similar to nuclear medicine imaging techniques to detect tissues, cells, or other material expressing the Abeta.

In certain embodiments, a binding molecule such as an antibody comprises an amino acid sequence or one or more moieties not normally associated with an antibody. Exemplary modifications are described in more detail below. For example, a single-chain fv antibody fragment can comprise a flexible linker sequence, or can be modified to add a functional moiety (e.g., PEG, a drug, a toxin, or a label).

An Abeta binding molecule polypeptide, e.g., an antibody polypeptide for use in the methods described herein can comprise, consist essentially of, or consist of a fusion protein. Fusion proteins are chimeric molecules that comprise, for example, an immunoglobulin antigen-binding domain with at least one target binding site, and at least one heterologous portion, i.e., a portion with which it is not naturally linked in nature. The amino acid sequences can normally exist in separate proteins that are brought together in the fusion polypeptide or they can normally exist in the same protein but are placed in a new arrangement in the fusion polypeptide. Fusion proteins can be created, for example, by chemical synthesis, or by creating and translating a polynucleotide in which the peptide regions are encoded in the desired relationship.

The term “heterologous” as applied to a polynucleotide or a polypeptide, means that the polynucleotide or polypeptide is derived from a distinct entity from that of the rest of the entity to which it is being compared. For instance, as used herein, a “heterologous polypeptide” to be fused to an antibody, or an antigen-binding fragment, variant, or analog thereof is derived from a non-immunoglobulin polypeptide of the same species, or an immunoglobulin or non-immunoglobulin polypeptide of a different species.

As discussed in more detail elsewhere herein, binding molecules, e.g., antibodies, or antigen-binding fragments, variants, or derivatives thereof for use in the methods described herein can further be recombinantly fused to a heterologous polypeptide at the N- or C-terminus or chemically conjugated (including covalent and non-covalent conjugations) to polypeptides or other compositions. For example, antibodies can be recombinantly fused or conjugated to molecules useful as labels in detection assays and effector molecules such as heterologous polypeptides, drugs, radionuclides, or toxins. See, e.g., PCT publications WO 92/08495; WO 91/14438; WO 89/12624; U.S. Pat. No. 5,314,995; and EP 396,387.

Binding molecules, e.g., antibodies, or antigen-binding fragments, variants, or derivatives thereof for use in the methods described herein can be composed of amino acids joined to each other by peptide bonds or modified peptide bonds, i.e., peptide isosteres, and can contain amino acids other than the 20 gene-encoded amino acids. Antibodies can be modified by natural processes, such as posttranslational processing, or by chemical modification techniques that are known in the art. Such modifications are described in basic texts and in more detailed monographs, as well as in a voluminous research literature. Modifications can occur anywhere in the antibody, including the peptide backbone, the amino acid side-chains and the amino or carboxyl termini, or on moieties such as carbohydrates. It will be appreciated that the same type of modification can be present in the same or varying degrees at several sites in a given antibody. Also, a given antibody can contain many types of modifications. Antibodies can be branched, for example, as a result of ubiquitination, and they can be cyclic, with or without branching. Cyclic, branched, and branched cyclic antibodies can result from posttranslation natural processes or can be made by synthetic methods. Modifications include acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cysteine, formation of pyroglutamate, formylation, gamma-carboxylation, glycosylation, GPI anchor formation, hydroxylation, iodination, methylation, myristoylation, oxidation, pegylation, proteolytic processing, phosphorylation, prenylation, racemization, selenoylation, sulfation, transfer-RNA mediated addition of amino acids to proteins such as arginylation, and ubiquitination. (See, e.g., Proteins—Structure And Molecular Properties, T. E. Creighton, W. H. Freeman and Company, New York 2nd Ed., (1993); Posttranslational Covalent Modification Of Proteins, B. C. Johnson, Ed., Academic Press, New York, pgs. 1-12 (1983); Seifter et al., Meth Enzymol 182:626-646 (1990); Rattan et al., Ann NY Acad Sci 663:48-62 (1992)).

The present description also provides for fusion proteins comprising a binding molecule, e.g., an antibody, or antigen-binding fragment, variant, or derivative thereof, and a heterologous polypeptide. In one embodiment, a fusion protein for use in the methods described herein comprises, consists essentially of, or consists of, a polypeptide having the amino acid sequence of any one or more of the VH regions of an antibody or the amino acid sequence of any one or more of the VL regions of an antibody or fragments or variants thereof, and a heterologous polypeptide sequence. In another embodiment, a fusion protein for use in the diagnostic and treatment methods disclosed herein comprises, consists essentially of, or consists of a polypeptide having the amino acid sequence of any one, two, three of the VH-CDRs of an antibody, or fragments, variants, or derivatives thereof, or the amino acid sequence of any one, two, three of the VL-CDRs of an antibody, or fragments, variants, or derivatives thereof, and a heterologous polypeptide sequence. In one embodiment, the fusion protein for use in the methods described herein comprises a polypeptide having the amino acid sequence of a VH-CDR3 of an antibody, or fragment, derivative, or variant thereof, and a heterologous polypeptide sequence, which fusion protein specifically binds to Abeta. In another embodiment, a fusion protein for use in the methods described herein comprises a polypeptide having the amino acid sequence of at least one VH region of an antibody and the amino acid sequence of at least one VL region of an antibody or fragments, derivatives or variants thereof, and a heterologous polypeptide sequence. The VH and VL regions of the fusion protein can correspond to a single source antibody (or scFv or Fab fragment) that specifically binds Abeta. In yet another embodiment, a fusion protein for use in the diagnostic and treatment methods disclosed herein comprises a polypeptide having the amino acid sequence of any one, two, three or more of the VH CDRs of an antibody and the amino acid sequence of any one, two, three or more of the VL CDRs of an antibody, or fragments or variants thereof, and a heterologous polypeptide sequence. Two, three, four, five, six, or more of the VH-CDR(s) or VL-CDR(s) can correspond to a single source antibody (or scFv or Fab fragment). Nucleic acid molecules encoding these fusion proteins are contemplated.

As discussed elsewhere herein, a moiety that enhances the stability or efficacy of an Abeta binding molecule, e.g., a binding polypeptide, e.g., an antibody or immunospecific fragment thereof can be conjugated. For example, in one embodiment, PEG can be conjugated to the Abeta binding molecules to increase their half-life in vivo. Leong, S. R., et al., Cytokine 16:106 (2001); Adv. in Drug Deliv. Rev. 54:531 (2002); or Weir et al., Biochem. Soc. Transactions 30:512 (2002).

Moreover, antibodies, or antigen-binding fragments, variants, or derivatives thereof for use in the methods described herein can be fused to marker sequences, such as a peptide to facilitate their purification or detection. In some embodiments, the marker amino acid sequence is a hexa-histidine peptide, such as the tag provided in a pQE vector (QIAGEN, Inc., 9259 Eton Avenue, Chatsworth, Calif., 91311), among others, many of which are commercially available. As described in Gentz et al., Proc. Natl. Acad. Sci. USA 86:821-824 (1989), for instance, hexa-histidine provides for convenient purification of the fusion protein. Other peptide tags useful for purification include, but are not limited to, the “HA” tag, which corresponds to an epitope derived from the influenza hemagglutinin protein (Wilson et al., Cell 37:767 (1984)) and the “flag” tag.

Fusion proteins can be prepared using methods that are known in the art (see for example U.S. Pat. Nos. 5,116,964 and 5,225,538). The precise site at which the fusion is made can be selected empirically to optimize the secretion or binding characteristics of the fusion protein. DNA encoding the fusion protein is then transfected into a host cell for expression.

Antibodies for use in the methods described herein can be used in non-conjugated form or can be conjugated to at least one of a variety of molecules, e.g., to improve the therapeutic properties of the molecule, to facilitate target detection, or for imaging or therapy of the patient. Antibodies, or antigen-binding fragments, variants, or derivatives thereof for use in the methods described herein can be labeled or conjugated either before or after purification, when purification is performed.

In particular, a binding molecule, e.g., an antibody, or antigen-binding fragment, variant, or derivative thereof for use in the diagnostic and treatment methods disclosed herein can be conjugated to cytotoxins (such as radioisotopes, cytotoxic drugs, or toxins), therapeutic agents, cytostatic agents, biological toxins, prodrugs, peptides, proteins, enzymes, viruses, lipids, biological response modifiers, pharmaceutical agents, immunologically active ligands (e.g., lymphokines or other antibodies wherein the resulting molecule binds to both the neoplastic cell and an effector cell such as a T cell), or PEG.

The above described fusion protein can further comprise a cleavable linker or cleavage site for proteinases. These spacer moieties, in turn, can be either insoluble or soluble (Diener et al., Science 231 (1986), 148) and can be selected to enable drug release from the antigen at the target site. Examples of therapeutic agents that can be coupled to the antibodies and antigens for immunotherapy are drugs, radioisotopes, lectins, and toxins. In using radioisotopically conjugated antibodies or antigens for, e.g., immunotherapy, certain isotopes can be selected depending on such factors as leukocyte distribution as well as stability and emission. Depending on the autoimmune response, some particular emitters can be selected. In general, alpha and beta particle emitting radioisotopes are used in immunotherapy. Short range, high energy a emitters such as ²¹²Bi can be used. Examples of radioisotopes that can be bound to the antibodies or antigens for therapeutic purposes include, but are not limited to ¹²⁵I, ¹³¹I, ⁹⁰Y, ⁶⁷Cu, ⁶⁴Cu, ²¹²Bi, ²¹²At, ²¹¹Pb, ⁴⁷Sc, ¹⁰⁹Pd and ¹⁸⁸Re. Other therapeutic agents that can be coupled to the Abeta binding molecule, e.g., antibody or antigen binding fragment thereof, as well as ex vivo and in vivo therapeutic protocols, are known, or can be easily ascertained, by one of skill in the art. Wherever appropriate, one of skill in the art can use a polynucleotide encoding any one of the above described antibodies, antigens or the corresponding vectors instead of the proteinaeous material itself.

One of skill in the art will appreciate that conjugates can also be assembled using a variety of techniques depending on the selected agent to be conjugated. For example, conjugates with biotin are prepared e.g. by reacting a binding polypeptide with an activated ester of biotin such as the biotin N-hydroxysuccinimide ester. Similarly, conjugates with a fluorescent marker can be prepared in the presence of a coupling agent, e.g. those listed herein, or by reaction with an isothiocyanate, such as fluorescein-isothiocyanate. Conjugates of the antibodies, or antigen-binding fragments, variants, or derivatives thereof are prepared in an analogous manner.

The present description further encompasses antibodies, or antigen-binding fragments, variants, or derivatives thereof for use in the methods described herein conjugated to a diagnostic or therapeutic agent. The antibodies can be used diagnostically to, for example, monitor the development or progression of a neurological disease as part of a clinical testing procedure to, e.g., determine the efficacy of a given treatment and/or prevention regimen. Detection can be facilitated by coupling the antibody, or antigen-binding fragment, variant, or derivative thereof to a detectable substance. Examples of detectable substances include various enzymes, prosthetic groups, fluorescent materials, luminescent materials, bioluminescent materials, radioactive materials, positron emitting metals using various positron emission tomographies, and nonradioactive paramagnetic metal ions. See, e.g., U.S. Pat. No. 4,741,900 for metal ions that can be conjugated to antibodies for use as diagnostics. Examples of suitable enzymes include horseradish peroxidase, alkaline phosphatase, beta-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin; and examples of suitable radioactive material include ¹²⁵I, ¹³¹I, ¹¹¹In or ⁹⁹Tc.

An antibody, or antigen-binding fragment, variant, or derivative thereof also can be detectably labeled by coupling it to a chemiluminescent compound. The presence of the chemiluminescent-tagged antibody is then determined by detecting the presence of luminescence that arises during the course of a chemical reaction. Examples of particularly useful chemiluminescent labeling compounds are luminol, isoluminol, theromatic acridinium ester, imidazole, acridinium salt and oxalate ester.

One of the ways in which an antibody, or antigen-binding fragment, variant, or derivative thereof can be detectably labeled is by linking the same to an enzyme and using the linked product in an enzyme immunoassay (EIA) (Voller, A., “The Enzyme Linked Immunosorbent Assay (ELISA)” Microbiological Associates Quarterly Publication, Walkersville, Md., Diagnostic Horizons 2:1-7 (1978)); Voller et al., J. Clin. Pathol. 31:507-520 (1978); Butler, J. E., Meth. Enzymol. 73:482-523 (1981); Maggio, E. (ed.), Enzyme Immunoassay, CRC Press, Boca Raton, Fla., (1980); Ishikawa, E. et al., (eds.), Enzyme Immunoassay, Kgaku Shoin, Tokyo (1981). The enzyme, which is bound to the antibody will react with an appropriate substrate, such as a chromogenic substrate, in such a manner as to produce a chemical moiety that can be detected, for example, by spectrophotometric, fluorimetric or by visual means. Enzymes that can be used to detectably label the antibody include, but are not limited to, malate dehydrogenase, staphylococcal nuclease, delta-5-steroid isomerase, yeast alcohol dehydrogenase, alpha-glycerophosphate, dehydrogenase, triose phosphate isomerase, horseradish peroxidase, alkaline phosphatase, asparaginase, glucose oxidase, beta-galactosidase, ribonuclease, urease, catalase, glucose-6-phosphate dehydrogenase, glucoamylase and acetylcholinesterase. Additionally, the detection can be accomplished by colorimetric methods that employ a chromogenic substrate for the enzyme. Detection can also be accomplished by visual comparison of the extent of enzymatic reaction of a substrate in comparison with similarly prepared standards.

Detection can also be accomplished using any of a variety of other immunoassays. For example, by radioactively labeling the antibody, or antigen-binding fragment, variant, or derivative thereof, it is possible to detect the antibody through the use of a radioimmunoassay (RIA) (see, for example, Weintraub, B., Principles of Radioimmunoassays, Seventh Training Course on Radioligand Assay Techniques, The Endocrine Society, (March, 1986)), which is incorporated by reference herein). The radioactive isotope can be detected by means including, but not limited to, a gamma counter, a scintillation counter, or autoradiography.

An antibody, or antigen-binding fragment, variant, or derivative thereof for use in the methods described herein can also be detectably labeled using fluorescence emitting metals such as 152Eu, or others of the lanthanide series. These metals can be attached to the antibody using such metal chelating groups as diethylenetriaminepentacetic acid (DTPA) or ethylenediaminetetraacetic acid (EDTA).

Techniques for conjugating various moieties to an antibody, or antigen-binding fragment, variant, or derivative thereof are known to one of skill in the art.

VII. Compositions and Methods of Use

Compositions comprising the aforementioned Abeta binding molecule, e.g., antibody or antigen binding fragment thereof or chemical derivatives thereof, or the polynucleotide, vector or cell can be used in the methods described herein. The compositions can further comprise a pharmaceutically acceptable carrier. The term “chemical derivative” describes a molecule that contains additional chemical moieties that are not normally a part of the base molecule. Such moieties can improve the solubility, half-life, absorption, etc. of the base molecule. Alternatively the moieties can attenuate undesirable side effects of the base molecule or decrease the toxicity of the base molecule. Said pharmaceutical composition can be designed to be administered intravenously, intramuscularly, subcutaneously, intraperitoneally, intranasally, parenterally or as an aerosol; see also infra.

The present description also provides a pharmaceutical and diagnostic, respectively, pack or kit comprising one or more containers filled with one or more of the above described ingredients, e.g. Abeta binding molecule, antibody or binding fragment thereof, antigen, polynucleotide, vector or cell. Associated with such container(s) can be a notice in the form prescribed by a governmental agency regulating the manufacture, use or sale of pharmaceuticals or biological products, which notice reflects approval by the agency of manufacture, use or sale for human administration. In addition or alternatively the kit comprises reagents and/or instructions for use in appropriate diagnostic assays. The composition, e.g. kit is of course particularly suitable for the diagnosis, prevention and treatment of a disease, disorder, injury or condition, as defined above.

The pharmaceutical compositions can be formulated according to methods known in the art; see for example Remington: The Science and Practice of Pharmacy, 21^{st} Ed. (Remington and Beringer, Lippincott Williams and Wilkins, 2006). Examples of suitable pharmaceutical carriers are well known in the art and include phosphate buffered saline solutions, water, emulsions, such as oil/water emulsions, various types of wetting agents, sterile solutions etc. Compositions comprising such carriers can be formulated by well known conventional methods. These pharmaceutical compositions can be administered to the subject at a suitable dose. Administration of the suitable compositions can be effected by different ways, e.g., by intravenous, intraperitoneal, subcutaneous, intramuscular, topical, parenterally, intranasally or intradermal administration. Aerosol formulations such as nasal spray formulations include purified aqueous or other solutions of the active agent with preservative agents and isotonic agents. Such formulations can be adjusted to a pH and isotonic state compatible with the nasal mucous membranes. Formulations for rectal or vaginal administration can be presented as a suppository with a suitable carrier.

Furthermore, whereas the present methods include direct administration of a compound to a target tissue, e.g., by drilling a small hole in the skull to administer a drug or using a pump such as an Ommaya pump, in one aspect, an Abeta binding molecule, e.g., an antibody or antibody used as a drug can cross the blood-brain barrier, which allows for indirect administration, e.g., intravenous or oral administration.

The dosage regimen will be determined by the attending physician and clinical factors. As is well known in the medical arts, dosages for any one patient depends upon many factors, including the patient's size, body surface area, age, the particular compound to be administered, sex, time and route of administration, general health, and other drugs being administered concurrently. A typical dose can be, for example, in the range of 0.001 to 1000 μg; however, doses below or above this exemplary range are envisioned, especially considering the aforementioned factors. Generally, the dosage can range, e.g., from about 0.0001 to 100 mg/kg, and more usually 0.01 to 5 mg/kg (e.g., 0.02 mg/kg, 0.25 mg/kg, 0.5 mg/kg, 0.75 mg/kg, 1 mg/kg, 2 mg/kg, etc.), of the host body weight. For example dosages can be 1 mg/kg body weight or 10 mg/kg body weight or within the range of 1-10 mg/kg, or at least 1 mg/kg. Doses intermediate in the above ranges are also intended to be within the scope of the methods described herein. Subjects can be administered such doses daily, on alternative days, weekly or according to any other schedule determined by empirical analysis. An exemplary treatment entails administration in multiple dosages over a prolonged period, for example, of at least six months. Additional exemplary treatment regimes entail administration once per every two weeks or once a month or once every 3 to 6 months. Exemplary dosage schedules include 1-10 mg/kg or 15 mg/kg on consecutive days, 30 mg/kg on alternate days or 60 mg/kg weekly. In some methods, two or more monoclonal antibodies with different binding specificities are administered simultaneously, in which case the dosage of each antibody administered falls within the ranges indicated.

A typical dose can be, for example, from about 0.01 mg to about 500 mg, from about 0.05 mg to about 250 mg, from about 0.10 mg to about 150 mg, from about 0.25 mg to about 100 mg, from about 0.5 mg to about 10 mg, from about 1 mg to about 5 mg, or about 5 mg. A typical dose can also be, for example, about 0.01 mg to about 0.10 mg, from about 0.10 mg to about 0.50 mg, from about 0.50 mg to about 1.0 mg, from about 1.0 mg to about 10 mg, from about 5 mg to about 50 mg, or from about 10 mg to about 500 mg.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives can also be present such as, for example, antimicrobials, anti-oxidants, chelating agents, and inert gases and the like. Furthermore, the pharmaceutical composition can comprise further agents such as dopamine or psychopharmacologic drugs, depending on the intended use of the pharmaceutical composition. Furthermore, the pharmaceutical composition can also be formulated as a vaccine, for example, if the pharmaceutical composition comprises an anti-Abeta antibody for passive immunization.

Furthermore, the pharmaceutical composition can comprise further agents such as interleukins or interferons depending on the intended use of the pharmaceutical composition. For example, in the treatment of Alzheimer's disease the additional agent can selected from the group consisting of small organic molecules, inorganic molecules, anti-Abeta antibodies, nucleic acids, peptides, and combinations thereof. Other agents, in combination with the Abeta binding molecules, can be simultaneously or sequentially administered. A therapeutically effective dose or amount refers to that amount of the active ingredient sufficient to ameliorate the symptoms or condition. Therapeutic efficacy and toxicity of such compounds can be determined by standard pharmaceutical procedures in cell cultures or experimental animals, e.g., ED₅₀ (the dose therapeutically effective in 50% of the population) and LD₅₀ (the dose lethal to 50% of the population). The dose ratio between therapeutic and toxic effects is the therapeutic index, and it can be expressed as the ratio, LD₅₀/ED₅₀. The therapeutic agent in the composition can be present in an amount sufficient to restore normal behavior and/or cognitive properties in case of Alzheimer's disease.

The pharmaceutical compositions can be used for the treatment of neurological diseases, disorders, injuries or conditions including but not limited to Alzheimer's disease, Down's Syndrome, head trauma, dementia pugilistica, chronic traumatic encephalopathy (CTE), chronic boxer's encephalopathy, traumatic boxer's encephalopathy, boxer's dementia, punch-drunk syndrome, amyloid deposition associated with aging, mild cognitive impairment, cerebral amyloid angiopathy, Lewy body dementia, vascular dementia, mixed dementia, multi-facet dementia, hereditary cerebral hemorrhage with amyloidosis Dutch type and Icelandic type, glaucoma, Parkinson's disease, Huntington's disease, Creutzfeldt-Jakob disease, cystic fibrosis, or Gaucher's disease and inclusion body myositis. Unless stated otherwise, the terms neurodegenerative, neurological or neuropsychiatric are used interchangeably herein.

Progress can be monitored by periodic assessment. Detection of treatment efficacy in humans can be performed by known methods, e.g., computed tomography (CT), position emission tomography (PET), for example with KB, FDG or 18F-FDDNP, magnetic resonance imaging (MRI), and sonography. Detection of treatment efficacy in humans can also be performed using behavioral assays.

These and other embodiments are disclosed and encompassed by the description and examples. Further literature concerning any one of the materials, methods, uses and compounds to be employed in accordance with the methods described herein can be retrieved from public libraries and databases, using for example electronic devices. For example the public database “Medline,” which is hosted by the National Center for Biotechnology Information and/or the National Library of Medicine at the National Institutes of Health can be used. Further databases and web addresses, such as those of the European Bioinformatics Institute (EBI), which is part of the European Molecular Biology Laboratory (EMBL) are known to one of skill in the art and can also be obtained using internet search engines. An overview of patent information in biotechnology and a survey of relevant sources of patent information useful for retrospective searching and for current awareness is given in Berks, TIBTECH 12 (1994), 352-364.

The above disclosure generally describes the methods contemplated herein. Unless otherwise stated, a term as used herein is given the definition as provided in the Oxford Dictionary of Biochemistry and Molecular Biology, Oxford University Press, 1997, revised 2000 and reprinted 2003, ISBN 0 19 850673 2. Several documents are cited throughout the text of this specification. The contents of all cited references (including literature references, issued patents, published patent applications as cited throughout this application and manufacturer's specifications, instructions, etc) are hereby expressly incorporated by reference; however, there is no admission that any document cited is prior art as to the present invention.

A more complete understanding can be obtained by reference to the following specific examples that are provided herein for purposes of illustration only and are not intended to limit the scope of the invention.

### EXAMPLES

The examples that follow further illustrate the invention, but should not be construed to limit the scope of the invention in any way. Detailed descriptions of conventional methods, such as those employed herein can be found in the cited literature; see also “The Merck Manual of Diagnosis and Therapy” Seventeenth Ed. ed by Beers and Berkow (Merck & Co., Inc. 2003).

The practice of the present invention will employ, unless otherwise indicated, conventional techniques of cell biology, cell culture, molecular biology, transgenic biology, microbiology, recombinant DNA, and immunology, which are within the skill of one in the art. For further elaboration of general techniques useful in the practice of this invention, the practitioner can refer to standard textbooks and reviews in cell biology and tissue culture; see also the references cited in the examples. General methods in molecular and cellular biochemistry can be found in such standard textbooks as Molecular Cloning: A Laboratory Manual, 3rd Ed. (Sambrook et al., Harbor Laboratory Press 2001); Short Protocols in Molecular Biology, 4th Ed. (Ausubel et al. eds., John Wiley & Sons 1999); DNA Cloning, Volumes I and II (Glover ed., 1985); Oligonucleotide Synthesis (Gait ed., 1984); Nucleic Acid Hybridization (Hames and Higgins eds. 1984); Transcription And Translation (Hames and Higgins eds. 1984); Culture Of Animal Cells (Freshney and Alan, Liss, Inc., 1987); Gene Transfer Vectors for Mammalian Cells (Miller and Calos, eds.); Current Protocols in Molecular Biology and Short Protocols in Molecular Biology, 3rd Edition (Ausubel et al., eds.); and Recombinant DNA Methodology (Wu, ed., Academic Press). Gene Transfer Vectors For Mammalian Cells (Miller and Calos, eds., 1987, Cold Spring Harbor Laboratory); Methods In Enzymology, Vols. 154 and 155 (Wu et al., eds.); Immobilized Cells And Enzymes (IRL Press, 1986); Perbal, A Practical Guide To Molecular Cloning (1984); the treatise, Methods In Enzymology (Academic Press, Inc., N.Y.); Immunochemical Methods In Cell And Molecular Biology (Mayer and Walker, eds., Academic Press, London, 1987); Handbook Of Experimental Immunology, Volumes I-IV (Weir and Blackwell, eds., 1986). Protein Methods (Bollag et al., John Wiley & Sons 1996); Non-viral Vectors for Gene Therapy (Wagner et al. eds., Academic Press 1999); Viral Vectors (Kaplitt & Loewy eds., Academic Press 1995); Immunology Methods Manual (Lefkovits ed., Academic Press 1997); and Cell and Tissue Culture: Laboratory Procedures in Biotechnology (Doyle & Griffiths, John Wiley & Sons 1998). Reagents, cloning vectors and kits for genetic manipulation referred to in this disclosure are available from commercial vendors such as BioRad, Stratagene, Invitrogen, Sigma-Aldrich, and ClonTech. General techniques in cell culture and media collection are outlined in Large Scale Mammalian Cell Culture (Hu et al., Curr. Opin. Biotechnol. 8 (1997), 148); Serum-free Media (Kitano, Biotechnology 17 (1991), 73); Large Scale Mammalian Cell Culture (Curr. Opin. Biotechnol. 2 (1991), 375); and Suspension Culture of Mammalian Cells (Birch et al., Bioprocess Technol. 19 (1990), 251); Extracting information from cDNA arrays, Herzel et al., CHAOS 11 (2001), 98-107.

The following experiments are illustrated and described with respect to antibody NI-101.11. However, the other antibodies of the NI 101 series, in particular NI 101.10 are structurally similar and thus can be expected to provide comparable results.

### Example 1

### Progression of AD-Like Pathology in APP/PS1 Mice

Transgenic mice over-expressing the Swedish mutation in the amyloid precursor protein (APP) develop amyloid-associated deficits that are greatly enhanced by the co-expression of mutant presenilin-1 (PS1) (Hsiao et al., Science 274:99-102 (1996); Holcomb et al., Nat. Med. 4:97-100 (1998)). Double transgenic APP/PS1 mice show behavioral impairment even before amyloid-beta (Abeta) accumulation, which begins at 3-4 months and worsens with age. To compare the Abeta neuropathology and its relative inflammation with the states of neurogenesis in this mouse model, APP/PS1 mice and same-aged wild type controls were compared.

For the analysis, cohorts of APP/PS1 mice and same-aged wild type controls were analyzed at three different stages: initial (3-4 months), established (11-12 months) and very advanced (17-18 months) pathology. Brain analyses and behavioral analyses were performed at each stage. The behavior analysis consisted of a Y-maze test, and the brain analyses included antibody staining to assess plaque formation and microglial number.

General Mouse Care and Sample Processing

All animal studies described herein were conducted under approval of the Swiss veterinary cantonal office (License Nr 48/08). Mice were bred in animal facilities using standard cages. Females were caged in groups of two to four, and males were caged individually to avoid social stress. Mice were kept in a 12-hour light cycle with food and water ad libitum. General nervous behavior and high mortality were generally associated with the transgenic mice.

In order to perform brain analyses, mice received an overdose of anesthesia (Ketamin/Xylaxine) and were transcardially perfused with 50 ml of saline, followed by 100 ml of ice-cold 4% paraformaldehyde in 0.1 M PBS, pH 7.4.

Brains were further processed as previously described by Iosif et al. (J Neurosci 26:9703-9712 (2006)). Briefly, after removal, brains were post-fixed overnight at 4° C. and consequently cryoprotected in 20% sucrose for at least 24 hours. Coronal sections 30 gm thick were cut using a sliding microtome, in 8 series, and stored at −20° C. in anti-freeze solution until use.

Antibody Staining

All sections were incubated for 1 hour at room temperature with potassium phosphate buffer solution (KPBS) containing 0.25% Triton-X-100 (KPBS-T) and 5% horse and/or goat serum to prevent nonspecific binding. Antibodies were diluted in the same solution but with a lower concentration of serum (2%). Sections were incubated with antibodies overnight at 4° C. After rinsing in KPBS-T, all sections were mounted on gelatin-coated glass slides and finally coverslipped with PVA-DABCO as anti-fading agent. Staining was then visualized by indirect immunofluorescence after 2 hours incubation at room temperature in the dark with secondary antibodies. The secondary antibodies used in the examples described herein were cyanine 3-conjugated donkey anti-rabbit/rat IgG antibody, FITC-conjugated goat anti-mouse/rabbit, cyanine 5-conjugated donkey anti-mouse IgG antibody, and for lectin staining FITC-conjugated streptavidin. The secondary antibodies were obtained from Jackson Immunoresearch (West Grove, USA) and used at 1:200 dilutions.

Immunostained samples were examined with an Inverted Leica DM IRE2 fluorescence and light microscope. The number of positive cells was counted in the granule cell layer (GCL) and within one cell diameter below this region in the subgranular zone (SGZ) (referred to together as SGZ/GCL). Numerical estimation of positive cells was done by applying the optical fractionator (Gundersen and Jensen, J. Microsc. 147:229-263 (1987)) with the help of newCAST software (Visiopharm, Copenhagen, Denmark). Briefly, at 100× magnification in oil, using a Leica DM4000 with motorized stage, the sampling area was defined as 15 μm of the total section thickness, with a guard zone of 2-3 μm above and below the dissector. For systematic sampling, the frame area was set to 3590 μm² with a step size of 226 μm. Six to eight equidistant coronal sections (240 μm apart) were analyzed per animal.

In order to evaluate plaque load, sections were stained with 6E10 antibody (1:1000; Signet, Cambridge, USA). The 6E10 antibody binds to both pre-amyloid and Abeta plaques. The area fraction (% Area), which is the area positive for the staining in a selected and defined area, was calculated using the “Measure” application of ImageJ (National Institutes of Health, U.S.A).

In order to assess microglial number, sections were stained with antibodies against ionized calcium-binding adaptor molecule 1 (“Iba1”) (1:1000; rabbit anti-Iba1; Wako Chemicals, Osaka, Japan). Iba1 is a macrophage/microglia-specific protein. The number of Iba1+ microglia surrounding the plaques was calculated using the “Analyze particles” application of ImageJ.

Y-Maze

The Y-maze test is a behavioral assay used to test spacial working memory. Y-maze tests were performed the day before mice were sacrificed. The test was performed by recording the number of spontaneous alterations during a 5-minute session in the Y-maze. Briefly, during the 5 minutes session, the sequence of arm entries was manually recorded by a blinded-experimenter whereas the ambulation was digitally recorded with a computer-aided video analysis system (EthoVision). Alternation was defined as successive entries into the three arms, in overlapping triplet sets. The percent alternation was calculated as the ratio of actual to possible alternations (defined as the total number of arm entries−2)×100%.

Statistical Analysis

All measurements were performed by an observer blind to animal identifications. For statistical analysis of the data described in this example and examples below, values presented are the average value of the group of study±standard error of the mean (SEM). For statistical analysis of all data the unpaired t test was applied, and for analysis of more than two groups the one-way ANOVA was used, followed by either Fisher's or Bonferroni's post hoc tests. Calculations were made with Statview 5.0, and conventional statistical significance was set at p≦0.05.

Results at 3-4 Months

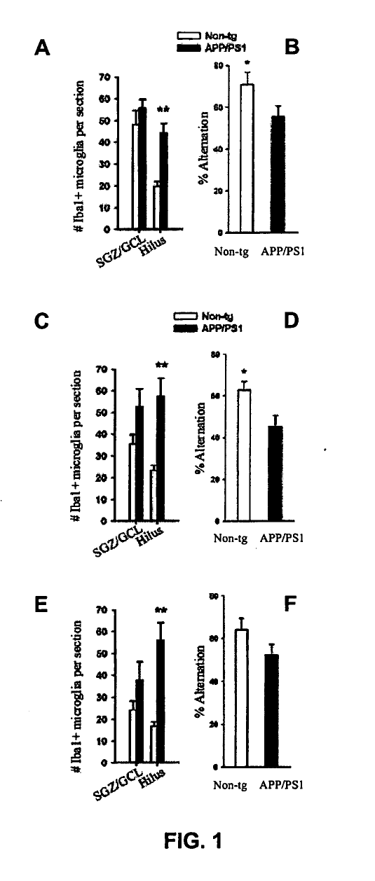

Plaque burden, as assessed by 6E10 antibody staining had just started in APP/PS1 mice at 3-4 months of age, and it covered approximately 3.5% of the brain. Plaque accumulation was visualized mainly in the cortex and was not yet visible in the dentate gyrus. However, differences in the dentate gyrus were apparent in Iba1 staining. APP/PS1 mice showed a higher number of Iba1 positive microglia. While a slight increase in Iba1 positive microglia was observed in the subgranular zone and granular cell layer (SGZ/GCL), a significant increase in Iba1 positive cells was observed in the hilus (FIG. 1A).

Differences between the APP/PS1 and wild-type mice were also observed in behavior analysis. In agreement with previous reports, 3-month old APP/PS1 mice already performed worse than controls in the Y-maze task (FIG. 1B). They showed significantly decreased percent alterations. No differences in the number of arm entries were detected among the groups (wild type: 31.50±2.6, APP/PS1: 29.3±4.2).

Results at 11-12 Months

At 11-12 months, APP/PS1 mice showed a dramatic increase in plaque deposition, with approximately 31% of the brain diffusely covered with plaques. On average, 4.35% of hippocampal area was occupied by Abeta deposits, and plaques also appeared in the dentate gyrus at this stage.

The increase in plaques was accompanied by a remarkable inflammatory response, as defined by a large increase in the number of Iba1 positive microglia. The most dramatic increase in Iba1 positive cells was in the hilus, but an increase was also observed in the SGZ/GCL (FIG. 1C).

Consistent with the advanced state of the AD-like pathology, 11-12 month old APP/PS1 mice also showed impaired short-term memory performance as measured by Y-maze test (FIG. 1D, p<0.05). APP/PS1 mice showed a decreased percentage of alterations, but the number of arm entries did not differ among transgenic and wild-type controls (respectively, 30±6 and 32±2).

Results at 17-18 Months

At 17-18 months APP/PS1 mice, approximately 37% of the brain was covered with plaques, and the general health condition of the animals was heavily compromised. In addition, a further increase in plaque burden was observed in the hippocampus (6.47% of hippocampal area covered with plaques).

However, no corresponding increase in the number of Iba1 positive microglia was found in the dentate gyrus. The number was the same as that observed at the previous time point. This suggests that the chronic inflammation in APP/PS1 can exhaust the defensive response of the brain or that the microgliosis may have already reached saturation at one year of age. However, the hilus again showed a significantly higher number of Iba1 positive microglia in transgenic mice when compared to wild type (FIG. 1E, p<0.01).

Surprisingly, behavioral impairment in the Y-maze could no longer be detected at this age (FIG. 1F). It is probable that the severity of the pathology and the high variability between subjects at this age did not allow a conclusive analysis at this stage. The number of arm entries was the same for transgenic and wild type mice (respectively, 28±3 and 29±2).

These results indicate that aging-dependent amyloid deposition is accompanied by a steadily increasing and broad inflammation as well as by behavioral deficits. The results indicate that the hilus of the hippocampus, one of the two major neurogenic areas of the brain, can react to the disease onset even before plaque deposition reached the dentate gyrus.

### Example 2

### Neurogenesis is Differently Influenced by the Progression of AD-Like Pathology

In order to determine whether and how hippocampal neurogenesis was affected by the progression of AD-like pathology in the APP/PS1 mouse model, proliferation and survival of neurons in APP/PS1 mice was compared to that of same-aged wild-type controls.

A total of 42 gender balanced animals were used (3-4 months group: WT: n=7, TG: n=8; 11-12 months group: WT: n=7, TG n=7; 17-18 months group: WT: 8, TG n=5). Mice were injected one month prior to sacrifice with the thymidine-analog BrdU (50 mg/kg) twice daily for two weeks. BrdU administration was performed essentially as described in Iosif et al. (J. Neurosci. 26:9703-9712 (2006)). After sacrifice and brain-processing as described in Example 1, free-floating brain sections were denatured in 1 M HCl for 30 minutes at 65° C. and rinsed in KPBS (to neutralize pH) in order to prepare for BrdU visualization. Rat anti-BrdU antibody (1:100; Oxford Biotec, Oxfordshire, United Kingdom) was used in a cocktail with mouse anti-neuronal nuclear marker NeuN (1:100, clone MAB377, Chemicon, Temecula, USA), rabbit anti-astrocytes specific marker S100beta (1:5000, SWANT, Bellinzona, Switzerland) and/or rabbit polyclonal anti-C terminus of the transcription factor Zif268 (1:250, Santa Cruz Biotechnology, Santa Cruz, USA). Additional staining was performed using mouse anti polysialic acid-neural cell adhesion molecule (PSA-NCAM) (1:2000, AbCys, Paris, France) to identify immature neurons together with biotinylated-isolectin IB4, which is a marker for endothelial cells (1:200; Molecular Probes, Leiden, The Netherlands). Staining was performed as described in Example 1.

For staining of the mitotic marker phospho-histone H3 (pH3), free-floating sections were quenched with 3% H₂O₂ and 10% methanol in KPBS for 30 minutes at room temperature, rinsed in KPBS (to neutralize pH), preincubated as described in Example 1 and then incubated overnight at 4° C. with rabbit polyclonal anti-pH-3 (1:400, Upstate, Lake Placid, USA). Slides treated with anti-pH-3 antibodies were dehydrated before visualization. For visualization with the peroxidase technique, biotinylated goat anti-rabbit IgG (1.200 Jackson) was applied for 2 hrs followed by incubation with avidin-biotin-peroxidase complex (ABC, Vectastain Elite, Vector laboratories) for 1.5 hours. Finally the sections were treated with diaminobenzidine (0.5 mg/ml) and 3% hydrogen peroxide. For quantitation of stainings that were less frequently observed, such as pH-3, BrdU and PSA-NCAM staining in old animals, cells were counted in every eighth 30 μm section throughout the hippocampus in its rostrocaudal extension, and the sum of these counts was multiplied by eight to give absolute cells number in the SGZ/GCL. Triple stainings with BrdU, NeuN and S100beta were validated using a confocal scanning microscope (Leica TCS/SP2, Leica, Wetzlar, Germany) with Argon laser 488, HeNE laser 568 and HeNe laser 633 excitation filters.

Using the BrdU injection protocol described above, cells that incorporate the marker of DNA synthesis represent a mixture of continuously proliferating cells and committed post-mitotic cells. Because the pool of BrdU-immunoreactive (BrdU+) cells do not represent a true estimate of proliferating cells (for a review see Taupin, Brain Res. Rev. 53:198-214 (2007)), but rather a relative number corresponding to the injection paradigms applied, the proliferation was also quantitated using the M-phase mitosis marker phospho-histone H3 (pH-3). Short-term survival of neural precursor cells was assessed by the number of PSA-NCAM+ cells. Final phenotypic maturation of the newborn cells was determined by co-labeling of BrdU with the pan-neuronal marker NeuN (BrdU+/NeuN+) or with the astrocytic marker S100beta (BrdU+/S100beta+). Assessment of dendritic length and branching was performed essentially as described in Breunig et. al. (PNAS 104:20558-20563 (2007)) except PSA-NCAM staining was used instead of DCX. PSA-NCAM+ neurons were imaged using a 63× objective in water with a digital zoom of 2. On average, fifty to sixty Z-series of 0.25 μm were merged for analysis. Measurements of dendritic length were performed using the semi-automated software NeuronJ (described in Meijering et al. Cytometry 58A:167-176 (2004); http://www.imagescience.org/meijering/software/neuronj/), a plug-in for the ImageJ (http://rsb.info.nih.gov/ij/). Before proceeding with the specific analysis via the public platform ImageJ, all pictures were converted to 8-bit and adjusted to the same threshold within the group of study. For the assessment of dendritic length, n=20 cells per group for youngest mice, n=16 cells per group for the middle-aged mice and n=10 cells per group for the very old mice were analyzed.

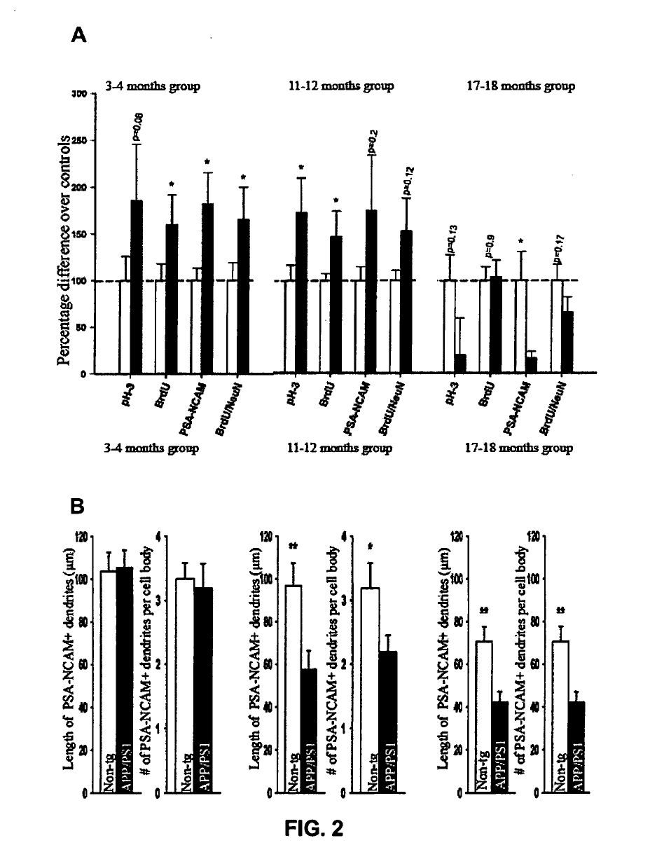

At 3-4 months, an increased number of proliferating cells (indicated by an increased number of BrdU+ cells and trend towards increased pH-3+ cells), of young new neurons (indicated by an increased number of PSA-NCAM+ cells) and of mature neurons (indicated by an increased number of BrdU+/NeuN+ cells) were found in APP/PS1 mice compared to controls (FIG. 2A). However, these cells did not appear to survive and/or differentiate into neurons at the 11-12 month stage. At 11-12 months, transgenic mice showed still higher proliferation (pH-3+ and BrdU+ cells), but they did not differ from wild-type controls in the number of young neurons (PSA-NCAM+ cells) and or mature neurons (BrdU+/NeuN+ cells) (FIG. 2A). At 17 to 18 months, diseased brains showed essentially no protective activity. Proliferation levels (pH-3+ cells) in transgenic mice were diminished, but not significantly different, from levels in control animals, while numbers of young neurons (PSA-NCAM+ cells) were significantly decreased in transgenic mice. However, at later stages of maturation (BrdU+/NeuN+ cells), no significant difference was detected among the two groups, probably due to the high variability found at this time point and to the aging of non-transgenic animals (FIG. 2A). In addition, the number of BrdU+/S100beta+ cells did not differ between APP/PS1 mice and controls at 3 months of age (TG: 65±23.9% versus WT: 100±31.8%), whereas later on double positive cells for these markers could no longer be detected in either wild type or transgenic mice.

Immature neurons express the neural cell adhesion molecule (PSA-NCAM), which is also detectable in the neuronal processes. This allows for immunolabeling of newborn neurons. In newborns, new cells start dendritic extension to the molecular layer, a process that will last for the next 5-6 weeks (van Praag et al., Nature 415:1030-1034 (2002)). In order to determine whether progressive Abeta-deposition would affect the morphological development of newly created granular cells, high-magnification confocal pictures of PSA-NCAM+ neurons were taken and analyzed with the freely available software NeuronJ. At early stages of the pathology, no differences were found between controls and APP/PS1 mice (FIG. 2B). However, at later stages with established and well diffused pathology, the new neurons presented consistently decreased dendritic length (40% shorter both at 11-12 months, p=0.007 and at 17-18 months, p=0.01) and a decreased number of processes per cell body compared healthy controls (31% less arborization at 11-12 months, p=0.04; no differences at 17-18 months) (FIG. 2C). An age-related withering of the immature neurons was also noticed in healthy controls. This fact could explain why the number of processes per cell body was the same among groups at the latest time point.

These data indicate that proliferation is initially stimulated in a model of AD with the morphology of the neurons initially remaining unaffected. Without being bound by theory, the progressive worsening of the disease may extinguish this self-repair action of the brain and disturb the morphology of the newly created cells. The data presented supra provide methods for identifying compounds that can modulate Alzheimer's disease and related diseases, for example, by administering a test compound to a model animal and detecting alterations in the behavioral and physical markers of disease.

### Example 3

### Passive Immunization as Treatment for AD-Like Pathology

Active immunization against Abeta with vaccines (Schenk et al., Nature 400:173-177 (1999); Morgan et al., Nature 408:982-985 (2000)) and passive immunization with chronic injections of anti-Abeta antibodies (Bard et al., Nat Med 6:916-919 (2000); DeMattos et al., Proc. Natl. Acad. Sci. USA 98:8850-8855 (2001)) have been shown to reduce plaque burden and improve cognition in different AD mouse models. Furthermore, a recent study reported stimulating neurogenesis using vaccinations with EFRH peptides (Becker et al., Proc. Natl. Acad. Sci. USA 104:1691-1696 (2007)).

The results described in the examples above demonstrate that the progression of neurodegeneration annuls the initial enhancement of neurogenesis seen in young transgenic animals, even though high proliferation is still detected at 11-12 months. Therefore, APP/PS1 mice were treated with a chimeric human-mouse monoclonal anti-Abeta antibody (“anti-Abeta”) and compared to mice treated with a control antibody of the same isotype (IgG2; “ct ab”) in order to determine if treatment with an antibody that can bind conformational Abeta could regress or slow the progression of AD-like pathology.

The anti-Abeta antibody used was a chimeric monoclonal antibody containing the fully human variable region of monoclonal antibody NI-101.11 (described in PCT Application PCT/EP2008/000053, filed Jan. 7, 2008, which is incorporated by reference in its entirety herein) and a mouse IgG2 constant region. NI-101.11 was isolated from B cells of a human Alzheimer's disease patient, and preferentially binds to conformational Abeta. The control antibody was raised against bovine herpes virus (clone 2H6-C2, obtained from European Collection of Cell Cultures (ECACC)).

Mice were treated for 3.5 months, starting at 8 months of age, with weekly injections (5 mg/kg, i.p.) of either the anti-Abeta antibody (7 mice) or the isotype control antibody (5 mice). The groups of mice were gender balanced. Plaque load, thioflavine S (ThioS) and cerebral amyloidosis angiopathy (CAA) levels were evaluated in the mice. Plaque load quantification was of diffuse plaque (6E10 staining). Compact plaque burden (ThioS) and CAA analyses were performed as described in Wilcock et al., (Nat. Protoc. 1:1591-1595 (2006)). Images from sequential sections were processed using the “Measure” application of ImageJ software to measure the area positive for staining (% Area) in a selected and defined area.

Staining results demonstrated that the anti-Abeta antibody reached the brain. Since the Fc part of the antibody against Abeta is murine, it could be detected using a Y3 fluorescent anti-mouse IgG antibody. Staining with the Y3 fluorescent anti-mouse IgG antibody revealed the presence of the anti-Abeta antibody around the plaques in all treated mice. In anti-Abeta treated APP/PS1 mice, plaque decoration was consistently found in defined areas of brain: from the lateral septal nuclei, through the formix, until the beginning of the thalamus. These areas are particularly rich in blood vessels, indicating that this could be the location where the antibody is released to the brain.

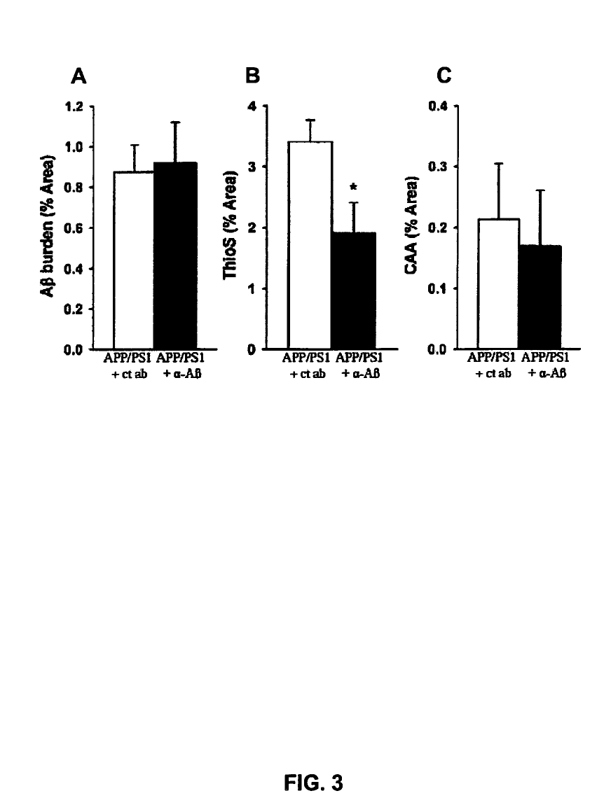

Animals treated with the anti-Abeta and control antibodies showed no difference in total Abeta burden as measured with 6E10, which stains both pre-amyloid and Abeta plaques (FIG. 3A). However, using thioflavine S, a fluorescent probe that only binds to dense-core amyloid-beta deposits, anti-Abeta treated animals showed a reduction of immunoreactivity to compact plaques compared to animals treated with the control antibody (FIG. 3B).

Some studies have linked immunotherapy to an increase in cerebral amyloidosis angiopathy (CAA)-related hemorrhage (Pfeifer et al., Science 298:1379 (2002); Racke et al., J. Neurosci. 25:629-636 (2005); Wilcock et al., Neuroscience 144:950-960 (2007)). In order to demonstrate that the anti-Abeta treatment did not decrease parenchymal amyloids while increasing vascular ones, CAA was quantitated in areas of the brain rich in blood vessels. No significant differences were detected between treated and control groups (FIG. 3C).

Microglia have been suggested to be activated by the opsonization of Abeta by antibodies that recognize Abeta deposited in amyloid plaques. (Bard et al., Nat. Med. 6:916-919 (2000)). To test this hypothesis Iba1+ microglia were quantitated in different areas of the brain. Control and treated APP/PS1 mice presented the same level of microgliosis (SGZ/GCL: APP/PS1+ ct ab: 50.5±8.6 vs. APP/PS1+anti-Abeta: 57.3±6.4 cells; hilus: APP/PS1+ct ab: 50.8±14.4 vs. APP/PS1+anti-Abeta: 55.9±7 cells; septum-formix-thalamus: APP/PS1+ct ab: 7.4±1.02% vs. APP/PS1+anti-Abeta: 8.9±1.06% area). The morphological subtypes were also evaluated. Ramified and intermediate shapes represent quiescent microglia, and amoeboid and round shapes represent activated microglia. There was no significant difference between the groups in the percentage of ramified- intermediate-, amoeboid- or round-shaped Iba1+ microglia in the dentate gyrus or in the septum (data shown below in Table 7). Furthermore, measurement of areas in the hippocampus and in the septum immunoreactive (CD11b+) for activated microglia did not reveal any differences between the groups (data shown below in Table 7; values in tables are means±S.E.M.).

TABLE 7

| | | Control | Abeta |
|---|---|---|---|
| | Microglia Type/Area | Antibody | Immunotherapy |
| Region | Fraction | (n = 5) | (n = 7) |
| SGZ/GCL | % ramified Iba1+ | 36.4 ± 22.5 | 24.4 ± 4.9 |
| | % intermediate Iba1+ | 49.3 ± 3.3 | 57 ± 4.8 |
| | % amoeboid Iba1+ | 13 ± 2.9 | 15.5 ± 2.7 |
| | % round Iba1+ | 1.3 ± 0.5 | 3.2 ± 1.4 |
| Hilus | % ramified Iba1+ | 20.5 ± 2.9 | 12.4 ± 3.0 |
| | % intermediate Iba1+ | 57.7 ± 7.2 | 59.8 ± 3.4 |
| | % amoeboid Iba1+ | 13.7 ± 4.2 | 21.5 ± 4.7 |
| | % round Iba1+ | 3.3 ± 1.1 | 6.3 ± 1.1 |
| Septum | % ramified Iba1+ | 18.3 ± 3.3 | 17.7 ± 3.4 |
| | % intermediate Iba1+ | 57.7 ± 7.2 | 49.8 ± 3.7 |
| | % amoeboid Iba1+ | 15.4 ± 5.3 | 21.4 ± 2.9 |
| | % round Iba1+ | 8.6 ± 3.2 | 11.1 ± 2.7 |
| Hippocampus | % area CD11b+ | 0.50 ± 0.10 | 0.65 ± 0.11 |
| Septum | % area CD11b+ | 0.84 ± 0.20 | 0.98 ± 0.31 |

Microglia can increase their proliferation rate during changes in activation state, but numbers of BrdU+/Iba1+ cells in the SGZ/GCL did not differ between anti-Abeta and control antibody treated mice (APP/PS1 + ct ab: 20.2 ± 2.1 vs. APP/PS1 + anti-Abeta: 21.6 ± 2.7).

According to the peripheral sink hypothesis (DeMattos et al., Proc. Natl. Acad. Sci. USA 98:8850-8855 (2001)), the binding of the antibody to Abeta in the plasma would induce a gradient efflux of Abeta from the brain to the periphery that would in turn result in lowered Abeta levels in the brain. To test this hypothesis ELISAs were used to analyze Abeta42 concentrations in the sera of anti-Abeta and control antibody treated APP/PS1 mice, untreated APP/PS1 mice and wild type mice. Among transgenic mice the concentration of Abeta42 was similar, though a modest increase was detected in the anti-Abeta group compared to the control antibody. All groups of APP/PS1 mice differed significantly from wild-type mice (APP/PS l+ct ab: 1413.5±96.6 pg/ml, APP/PS1+anti-Abeta: 1753±193.9 pg/ml, APP/PS1+PBS: 1378±97.3 pg/ml and WT: 383.8±125 pg/ml, p<0.001).

Finally, although the average weight of the mice treated with anti-Abeta and control antibody was the same (APP/PS1+ct ab: 29.9±1.4 g versus APP/PS1+anti-Abeta: 31.24±1.8 g), all animals treated with the anti-Abeta survived until the completion of the experiment. In contrast, two mice treated with the control antibody died.

These results demonstrate that antibodies to Abeta can enter the brain and are associated with a reduction of compact plaques. Furthermore, treatment with such antibodies does not appear to elicit the undesirable side effects of increasing parenchymal amyloidosis.

### Example 4

### Antibody Treatment Promotes Neurogenesis in APP/PS1 Mice

Further experiments were conducted to determine if passive immunization could succeed in promoting neurogenesis in mice presenting a very aggressive AD-like pathology.

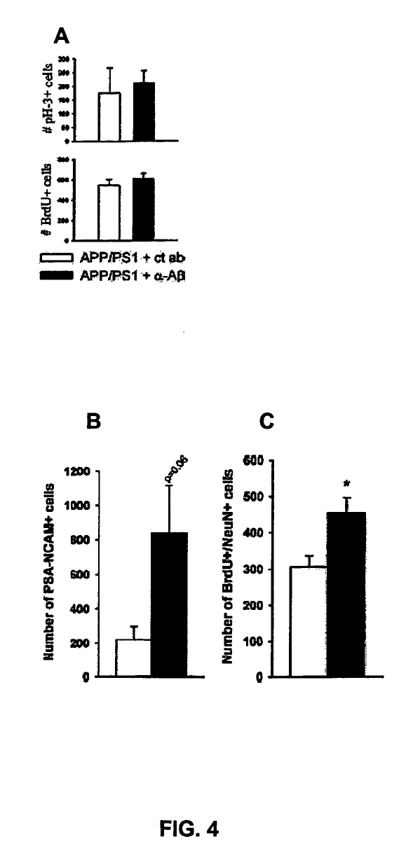

Up to one year of age, APP/PS1 mice consistently showed increased proliferation compared to wild type mice. Consequently, animals treated with anti-Abeta were examined to determine whether the treatment would intensify this phenomenon. Sections were stained to show the mitotic marker phospho-histone3 (pH-3). However, no differences in the number of pH-3+ and BrdU+ cells were found in the SGZ/GCL regions of brains of mice treated with anti-Abeta and with the control antibody (FIG. 4A). Nevertheless, numbers of immature (PSA-NCAM+) neurons were higher in APP/PS1 mice treated with anti-Abeta than with the control antibody (p=0.06; FIG. 4B). However, these results were not statistically significant, possibly as a result of the high variability. In contrast the numbers of mature (BrdU+/NeuN+) neurons was significantly higher in mice treated with anti-Abeta than with the control antibody (p<0.05) (FIG. 4C). (Fifty BrdU-positive cells from each animal (except for aged mice where all BrdU+ cells were considered) were analyzed for NeuN double labeling in the SGZ/GCL.) These results indicate that an anti-Abeta treatment can play a protective role that allows for the survival of the progenitors.

Gliogenesis, measured by the number of BrdU+/S100beta+ cells (astrocytes), was similar among anti-Abeta and control antibody treated groups (APP/PS1+ct ab: 56±12.13 versus APP/PS1+anti-Abeta: 43±12.72). This indicates that in the presence of sustained inflammation, microglia start to proliferate. Therefore the number of BrdU+ cells that were also Iba1+ was also evaluated. No differences were detected among the two groups (APP/PS1+ct ab: 20.2±2.1 versus APP/PS1+anti-Abeta: 21.6±2.7 BrdU+/Iba1+ cells).

Synaptic activity in the hippocampal dentate gyrus induces robust expression of the immediate early gene Zif268 (or Erg-1). Zif268 marks synaptic activation and its presence has been shown to be essential for the formation of long-term memories (Jones et al., Nat. Neurosci. 4:289-296 (2001)). If animals are not exposed to stimulations (Davis et al., Behav. Brain Res. 142:17-30 (2003); Bruel-Jungerman et al., J. Neurosci. 26:5888-5893 (2006)), Zif268 is not constitutively transcribed in the GCL. Therefore, few BrdU+/NeuN+ cells that were also positive for Zif268 could be identified. However, the colocalization of newly formed neurons (BrdU+/NeuN+) in the SGZ with Zif268+ demonstrates that the new cells can have synaptic activity. (Colocalization stainings were validated using a confocal scanning microscope (Leica TCS/SP2, Leica, Wetzlar, Germany) in orthogonal projections with Argon laser 488, HeNE laser 568 and HeNe laser 633 excitation filters.)

In order to increase Zif268 expression, mice were subjected, 10 minutes prior to anaesthesia, to a novel object by placing a quarter of an apple wrapped in tinfoil into the cages. A 10 minute exposure to a novel object has been shown by others to result in increases in Zif268 expression throughout the cortex and hippocampus (Kubik et al. Learn Mem. 14: 758-770 (2007)). Without exposure to the activation stimulus, Zif268 expression in APP/PS1 mice was low. The activation stimulus induced Zif268 expression throughout the neuronal population in the SGC/GCL in both the vehicle-treated (PBS, 100 μl/10 gm body weight, i.p.) and Abeta immunotherapy-treated mice. The expression of Zif268 in pre-existing neurons within the SGZ/GCL was similar in vehicle-treated and Abeta immunotherapy-treated APP/PS1 mice. The observation of Zif268 expression in BrdU+/NeuN+ cells demonstrates that new mature neurons in Abeta immunotherapy-treated mice can be functionally integrated.

Mice treated with the anti-Abeta and control antibodies were also behaviorally tested in Y-maze. Y-maze tests were performed the day before mice were sacrificed as described in Example 1, and mice were moved to an inverted light cycle room after the last BrdU injection. When compared to same aged untreated APP/PS1 mice, both anti-Abeta and control antibody treated groups improved (APP/PS1+ct ab: 59.98±0.03 versus APP/PS1+anti-Abeta: 56.23±0.02% Alternation; TG: 45.5±5.1, WT: 62.7±4.1% Alternation).

These data indicate that an anti-Abeta antibody can increase the number of mature neurons in an AD model and that new neurons can be integrated into functional neuronal circuits. Therefore anti-Abeta antibodies can promote neurogenesis.

### Example 5

### Passive Immunization Influences the Dendritic Arborization of New Granular Neurons and Synaptic Activity in the Molecular Layer of Hippocampus

Neuronal progenitors expressing PSA-NCAM still undergo fate decision (survival or death) and continue functional neuronal differentiation. Type-2a cells (Nestin+) are still silent, but they express functional GABA (A) and glutamate receptors (Wang et al., Mol. Cell. Neurosci. 29:181-189 (2005)). However, only one week later spineless dendrites of PSA-NCAM+ neuroblasts (Class C neurons) can receive GABAergic contacts (Esposito et al., J. Neurosci. 25:10074-10086 (2005)). One study proposed an activity-dependent regulation of adult neurogenesis, where sensing the surrounding neuronal network activities through GABA signaling could influence the integration of new neurons (Ge et al., Nature 439:589-93 (2006)). GABAergic functions were recently reported to be altered in a mouse model for AD as a compensatory mechanism for Abeta-related overexcitation (Palop et al., Neuron 55:697-711 (2007)). Therefore, experiments were conducted to determine whether antibody treatment could modulate survival of neuroblasts via creating a more favorable environment for these cells and thus promoting synaptic integration.

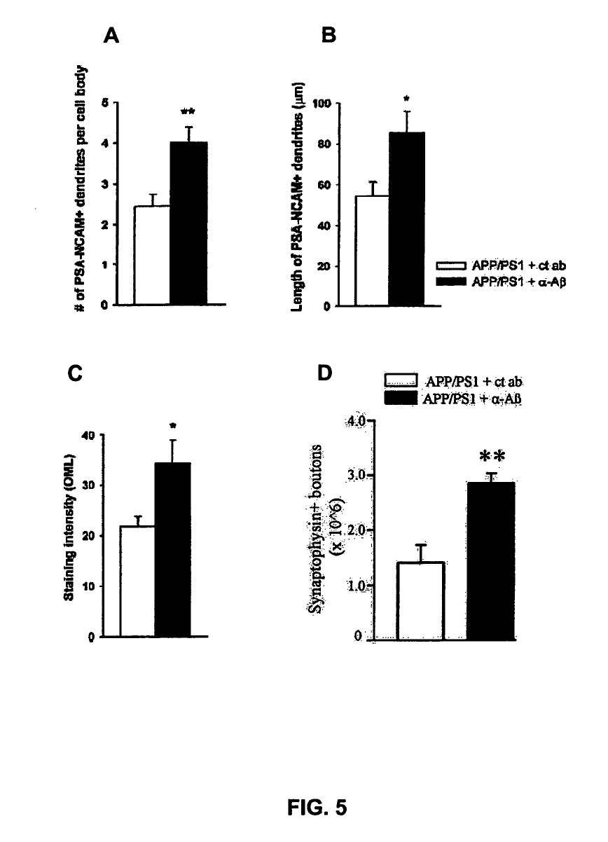

First, dendritic branching and length were assessed in APP/PS1 mice treated with antibody to Abeta and control antibody. Sections were stained with PSA-NCAM for the dendrite analysis. The number of dendrites per cell body was increased in anti-Abeta treated mice compared to controls (FIG. 5A). Dendritic length was assessed as described in Example 2. In these experiments, n=40 cells per group were analyzed. APP/PS1 mice treated with antibody to Abeta− PSA-NCAM+ neurons showed longer processes compared to controls (FIG. 5B).

Active and passive Abeta-immunization have been shown to prevent synaptic degeneration (Buttini et al., J. Neurosci. 25:9096-9101 (2005)). Therefore, in order to verify that such a protective effect could also be seen as a result of treatment with antibodies to Abeta, the levels of the presynaptic protein synaptophysin (“SYN”) were measured in the frontal neocortex and in the outer molecular layer (OML) of hippocampus, where granular neurons extend their dendrites toward the perforant path. For these experiments the mouse monoclonal anti-synaptophysin (1:200, Sigma, Saint Louis, USA) was used and staining was performed as described in Example 1. The intensity of the synaptic vesicle glycoprotein SYN signals was quantified as previously described (Buttini et al. (J. Neurosci. 25:9096-9101 (2005)) and Priller et al., J. Neurosci. 26:7212-7221 (2006)). For each zone of interest, four confocal images were taken at magnification 63× in water with digital zoom 2, and with Z-stacks of 0.25 μm. All pictures were taken during the same session. Sections from the treated and controls mice (n=5-7) were alternated during acquisition to obtain pixel intensity within a comparable range. Pictures were analyzed using the “Histogram” function of ImageJ (value reported: “mean”).

A significant effect was observed in the OML of doubly transgenic mice treated with Abeta antibodies (FIG. 5C), and a modest increase was also detected in the cortex (APP/PS1+ct ab: 36.16±5.4 versus APP/PS1+anti-Abeta: 45.8±4.2 Average Pixel Intensity). Mice treated with control antibody presented synaptophysin expression very similar to the untreated APP/PS1 mice (Cortex: 37.9±2.2, Hippocampus: 17.3±2.3 average pixel intensity), suggesting that differences were due to treatment with the Abeta antibodies. Synaptophysin expression in wild-type controls was significantly higher than expression in each of the transgenic groups other than in the hippocampus of Abeta-antibody treated APP/PS1 mice (cortex: 68±3.5, Hippocampus: 28.9±2.2 average intensity staining, p≦0.02 ANOVA followed by Fisher's PD).

Similar results were obtained by counting the numbers of SYN-positive boutons. In these experiments, synaptophysin-positive presynaptic terminals were counted bilaterally in the molecular layer of the hippocampal section using a Leica DM4000B microscope with a 100× oil objective, Olympus DP71 color digital camera, and newCAST software (Visiopharm, Copenhagen, Denmark) with the frame area set to 250 μm², with 200 μm sampling intervals at the x and y levels, and with the optical dissector constituting 2.5 μm-thick fractions of the total section thickness. The results demonstrated that Abeta-antibody treated APP/PS1 mice showed increased numbers of SYN-positive boutons in the molecular layer of the dentate gyrus (FIG. 5D).

These data indicate that treatment with anti-Abeta antibodies can increase dendrite length, promote dendritic arborization, and increase synaptic density in an AD model.

### Example 6

### Passive Immunization Increases Angiogenesis in the Dentate Gyrus

The blood-brain barrier (BBB) is disrupted in AD. Progressive deposition of Abeta along the walls of blood vessels reportedly leads to collapse of the vessels, hypoperfusion of the brain and finally neuronal injury (Zlokovic, Neuron 57:178-201 (2008)). Vascularization and neurogenesis are strictly correlated, with the first stimulating the latter (Jin et al., Proc. Natl. Acad. Sci. USA 99:11946-11950 (2002)). This indicates that vascularization may create favorable niches for the progenitors.

In APP/PS1 mice, many BrdU+ cells were located in the proximity of blood vessels. In addition, Abeta antibodies are released to the brain via the blood. In view of this information, experiments were conducted to test whether passive immunization could affect angiogensis. In these experiments, blood vessels were examined in treated and control animals using stereological evaluations, lectin staining and glucose transporter 1 (Glut1) staining. Glut1 is a glucose transporter that is highly expressed in the endothelial cells of barrier tissues, such as the blood-brain barrier. Stereological analyses were performed essentially as described in Lee et al., Brain Res. Bull. 65:317-322 (2005).

Estimation of blood vessel numbers in the dentate gyrus of antibodies-treated APP/PS1 mice was performed by applying the counting frame to estimate the number of points where a single capillary branches into two. Twice this number is equal to the number of capillary segments (Lee et al., Brain Res. Bull. 65:317-322 (2005)). Length of vessels was estimated counting the intersections between the vessels and computer-generated isotropic virtual planes (Larsen et al., J. Microsc. 191:238-248 (1998)). Glut1 staining was performed using rabbit anti-mouse Glut1 (1:500, Alpha Diagnostic, San Antonio, USA) as described in staining experiments above.

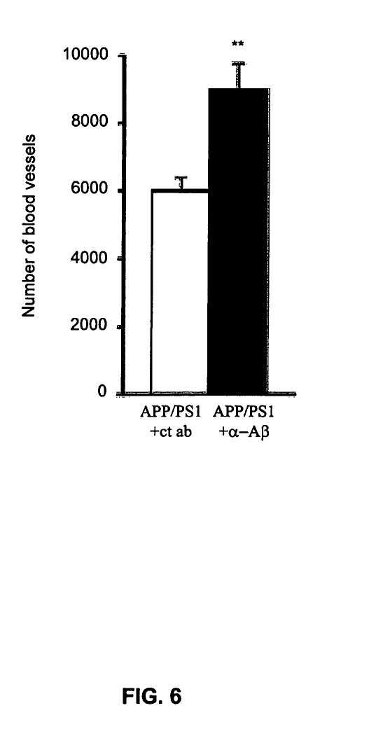

Stereological estimation of vessel length detected a trend towards an increase following antibody treatment (APP/PS1+ct ab: 0.17±0.03 vs. APP/PS1+anti-Abeta: 0.31±0.06, p=0.07). However, differences in blood vessels were apparent when assessed by either lectin or Glut1 staining. Amyloid deposits along the blood vessels downregulate the expression of Glut1. However, Glut1 levels were significantly higher in animals treated with the Abeta antibody than with the control antibody (APP/PS1+ct ab: 4271±568 versus APP/PS1+anti-Abeta: 8869±1570, p≦0.05). In addition, levels of lectin were significantly higher in animals treated with the Abeta antibody (APP/PS1+ct ab: 5991±384 versus APP/PS1+anti-Abeta: 8978±766, p<0.01). FIG. 6 shows an estimation of the number of blood vessels indicated by lectin staining.

Furthermore, confocal images of ThioS+ cells show Abeta deposition along the wall of the blood vessels and also demonstrated that Glut1 expression was disrupted in the presence of CAA. Colocalization of Abeta with Glut1 excludes the possibility that Glut1 was undetectable as a result of epitope masking by amyloid deposits.

These data indicate that there is indeed a higher number of blood vessels in APP/PS1 mice treated with the Abeta antibody than with the control antibody, and therefore, that treatment with the Abeta antibody increases angiogenesis.

### Example 7

### Abeta Immunotherapy Restores Dendritic Branching and Dendritic Spine Density in New Mature Neurons

In order to investigate whether Abeta immunotherapy affects the dendritic morphology of new granule cells when they have matured, the Abeta immunotherapy was repeated in mice that received retroviral injections into the dentate gyrus to label dividing cells with GFP.

To create the retrovirus for injection, an oncoretroviral vector derived from Moloney sarcoma virus and expressing GFP under the control of the Rous sarcoma virus promoter (MolRG) was used. The viral solution was prepared by cotransfection of HEK293FT cells (Invitrogen, Carlsbad, Calif.) using calcium-phosphate precipitation. After 48 hours, conditioned medium was concentrated by two sequential ultracentrifugations in sucrose gradients. Viral particles were resuspended in sterile PBS, aliquoted, and stored at −80° C. until use. Viral concentrations (10⁸⁻⁹ cfu/ml) were determined by serial dilutions on HEK293FT cells, and the number of GFP+ cells was counted 48 hours after infection using flow cytometry. For labeling of new neurons, mice were deeply anaesthetized with Ketamin/Xylaxine and given unilateral injections of the retroviral vectors (1.5 μl at 0.2 μl/min) into the dentate gyms (coordinates: 2 mm posterior and 1.5 mm lateral from bregma and 2.3 mm ventral from skull).

Fifteen mice were injected with the GFP-expressing retrovirus into the dentate gyms at 3 months of age, one month after the beginning of either passive Abeta immunization (5 mg/kg i.p.) (n=5 APP/PS1 mice (2 male; 3 female)) or vehicle treatment (PBS, 100 μl/10 gm body weight, i.p.) (n=5 APP/PS1 mice (2 male; 3 female); n=5 non-transgenic mice (2 male; 3 female)). Treatments, either passive Abeta immunization or vehicle treatment, were initiated at 2 months of age, occurred once a week, and lasted 2 months. Then, after a 5 minute transport from the animal facility within the same building, mice were placed under a hood for 30 minutes prior to anaesthesia and transcardial perfusion was performed.

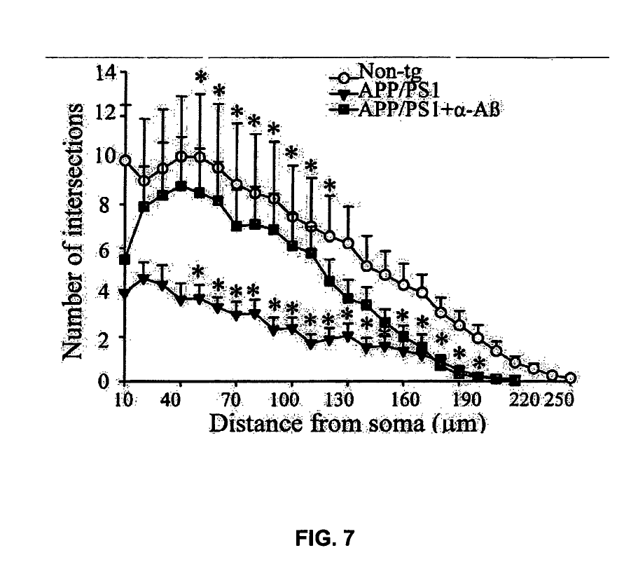

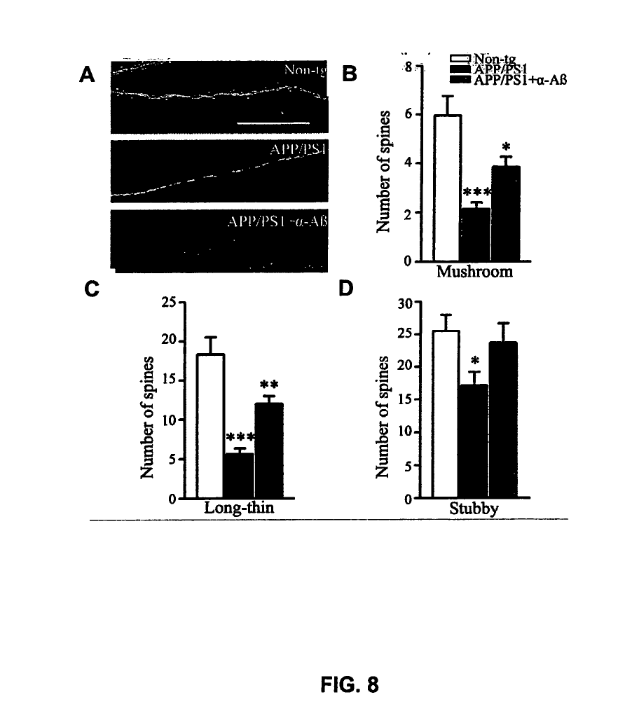

Following retrovirus injection, the dendritic length and branching of 5-week-old GFP+ new granule cells (n=15 per group) were analyzed using the Leica DM4000 microscope with 20× water objective and a digital zoom of 2 (1024×1024 pixel). On average 50-70 pictures with 0.5 μm steps were merged for analysis. High-magnification pictures for spine density were also taken using the Leica DM4000 microscope with a 63× water objective and a digital zoom of 6 (512×512 pixel). On average 100-130 pictures of 0.120 μm steps were merged for analysis. The autofluorescence of the labeled neurons was sufficiently strong so that anti-GFP staining was unnecessary. Z-stacks were deconvoluted to filter the signal to improve clarity of the images by increasing resolution, removing out-of-focus blur, and eliminating noise using the open source Huygens remote manager (http://hrm.sourceforge.net/). 3D reconstructions of deconvoluted z-stacks were performed with Imaris 6.1 (Bitplane, Zurich, Switzerland). After 3D reconstructions, Scholl analyses were done using the aforementioned software and its MatLab extensions (ImarisXT). In these analyses, intersections between dendrites and concentric circles, each circle with a 10 μm increment from the previous circle and centered around the soma, were counted to determine the total dendrite length and branching density along 40 μm segments (n=50 per group (FIG. 7). Spine classification (stubby, long-thin, or mushroom), according to the original classification of Peters and Kaiserman-Abramof (Am. J. Anat. 127:321-355 (1970)), was also performed (FIG. 8). Because spine density increases proportionally to the distance from the cell body (Sorra and Harris, Hippocampus 10:501-11 (2000)), for each GFP+ neuron used in the Scholl analysis, 1 and 2 dendritic segments were analyzed in the medial and external part of the outer molecular layer, respectively.

GFP+ granule cells were analyzed five weeks after the injection. Five weeks was considered to be a time sufficient for the newly born neurons to mature (van Praag et al., Nature 415:1030-1034 (2002) and Zhao et al., J. Neuroscience 26:3-11 (2006)). There were significantly fewer dendritic arborizations of the GFP+, new and mature granule cells in dentate gryi of vehicle-treated APP/PS1 mice than in dentate gryi of Abeta immunotherapy-treated APP/PS1 mice. The number of dendritic arborizations in Abeta immunotherapy-treated mice resembled the number of dendritic arborizations in new neurons born in non-transgenic mice.

Abeta immunotherapy also affected spine densities. Spines are the principle sites of excitatory synaptic transmission and can be classified according to their shapes and appearance. A significant reduction in long-thin spines, stubby spines, and mushroom spines was observed in all GFP+ cells in control antibody-treated mice compared to non-transgenic mice (FIG. 8). The Abeta immunotherapy reversed this loss of spines by about 50% for each type of spine (FIG. 8).

These data indicate that treatment with anti-Abeta antibodies can increase dendritic branching and can increase spine densities.

### Example 8

### The Presence of Cellular Prion Proteins on Newly Formed Neurons is Compatible with Role in Mediating Abeta-Related Toxicity

A role for cellular prion protein (PrP^{c}) in mediating synaptic toxicity of Abeta oligomers has been described (Lauren et al., Nature 457: 1128-1132 (2009)). To explore whether PrP^{c} is present on retrovirus-labeled GFP+ new neurons, the localization of PrP^{c} and GFP were observed. For this histological analysis, high magnification stacks (63x z8, 512×512 pixel, 0.16 z-step) were analyzed by using ColocImaris software. This software specifically identifies voxels that are positive in both fluorescent channels, thereby dramatically reducing artifacts generated by overlays in z-levels. PrP^{c} was present ubiquitously on cells throughout the brain. PrP^{c} was also present, at equal levels on pre-existing brain cells and on the dendrites of retrovirus-labeled GFP+ newly born neurons. PrP^{c} staining on dendrites of newly-born neurons had a patchy appearance consistent with its known cellular distribution (Hantman and Perl, J. Comp. Neurol. 492:90-100 (2005)), and approximately 5% of the analyzed dendritic surface of the newly-born neurons was co-stained for PrP^{c}. PrP^{c} was present at equal levels on dendrites of newly-born neurons in both non-transgenic mice and APP/PS1 mice. This suggests that the expression of APP and PS1 transgenes under the control of the PrP^{c} and PDGFbeta promoters, respectively, did not affect the co-localization of PrP^{c} with dendrites of newly-born neurons. Abeta immunotherapy had no obvious effect on the co-localization of PrP^{c} with dendrites of newly born neurons.

These data are consistent with a model in which PrP^{c} mediates Abeta-related toxicity in newly-born neurons in APP/PS1 mice. Accordingly, Abeta binding compounds can be used to block Abeta binding to PrPc and to modulate the signal transduction via PrPc and the resulting toxicity.

### Example 9

### Effects of Abeta Immunotherapy are Due to Abeta Aggregate Binding

In order to exclude the possibility that the effects of the Abeta immunotherapy were non-specific, the effect of antibody treatment on non-transgenic wild-type mice was also observed. In these experiments, non-transgenic wild-type mice that do not express human APP or have beta-amyloid pathology were used. These mice were treated with either vehicle (PBS, 100 μl/10 μm body weight, i.p. (n=5, 2 male and 3 female)) or anti-Abeta (5 mg/kg, i.p. (n=4, 1 male and 3 female) for 10 weeks before perfusion, starting at 2 months of age. Unlike in APP/PS1 mice, Abeta immunotherapy did not affect neurogenesis (BrdU/NeuN and PSA-NCAM) in non-transgenic mice. Quantification in the dentate gyri of markers for gliogenesis (BrdU/S100beta), proliferation (PCNA) and neuroinflammation (GFAP, Iba1, and CD11b) did not detect any differences between antibody and vehicle-treatments of non-transgenic wild-type mice. The results are summarized in Table 8 (values are means of ±S.E.M.; n=5 vehicle and n=4 Abeta immunotherapy; no significant differences between groups were observed).

TABLE 8

| Staining | Vehicle | Abeta immunotherapy |
|---|---|---|
| BrdU/NeuN | 2466 ± 260 | 2231 ± 657 |
| BrdU/S100beta | 29.5 ± 21 | 61 ± 52 |
| PSA-NCAM | 5698 ± 1689 | 5702 ± 681 |
| PCNA | 49.2 ± 6.4 | 38.4 ± 4.8 |
| GFAP | 8797 ± 1964 | 7433 ± 1194 |
| Iba1 | 3097 ± 497 | 3818 ± 355 |
| CD11b | 0 | 0 |

These data indicate that the binding of the antibody to human Abeta aggregates, rather than non-specific effects of IgG2, mediated the effects of Abeta immunotherapy on neurogenesis in the dentate gyrus.

Taken together, the data provided herein demonstrate that passive immunization can be effective for treating Alzheimer's disease and related diseases and can be useful for promoting neurogenesis.

1-75. (canceled)

76. A method of promoting neurogenesis, the method comprising administering to a subject in need thereof an effective amount of an Abeta binding molecule.

77. The method of claim 76, wherein the subject has an abnormal amyloid condition.

78. A method of promoting angiogenesis, the method comprising administering to a subject in need thereof an effective amount of an Abeta binding molecule.

79. A method of promoting synaptic density and/or activity, the method comprising administering to a subject in need thereof an effective amount of an Abeta binding molecule.

80. A method of promoting the dendritic arborization or an increase in dendritic spine density of a CNS neuron in a subject in need thereof, the method comprising administering to the subject an effective amount of an Abeta binding molecule.

81. The method of claim 80, wherein the CNS neuron is a granular neuron.

82. The method of claim 76, wherein the subject has an accumulation of Abeta.

83. The method of claim 76, wherein the Abeta binding molecule specifically binds a peptide selected from the group consisting of an Abeta₁₋₄₂ peptide, an Abeta₁₋₄₀ peptide, an Abeta₁₋₄₃ peptide, a fibrillar Abeta, a beta-amyloid fibril, a diffuse beta-amyloid deposits, a neoepitope of Abeta, and a beta-amyloid plaque.

84. The method of claim 76, wherein the Abeta binding molecule is an anti-Abeta antibody or antigen-binding fragment thereof and comprises a heavy chain variable region (VH) and a light chain variable region (VL).

85. The method of claim 84, wherein the (VH) of the anti-Abeta antibody or antigen-binding fragment thereof comprises an amino acid sequence at least 90% identical to a reference amino acid sequence selected from the group consisting of: SEQ ID NO: 4, SEQ ID NO: 6, SEQ ID NO: 10, SEQ ID NO: 14, SEQ ID NO: 39, SEQ ID NO: 42, and SEQ ID NO: 43.

86. The method of claim 85, wherein the VH of the anti-Abeta antibody or antigen-binding fragment thereof comprises an amino acid sequence selected from the group consisting of: SEQ ID NO: 4, SEQ ID NO: 6, SEQ ID NO: 10, SEQ ID NO: 14, SEQ ID NO: 39, SEQ ID NO: 42, and SEQ ID NO: 43.

87. The method of claim 84, wherein the VL of the anti-Abeta antibody or antigen-binding fragment thereof comprises an amino acid sequence at least 90% identical to a reference amino acid sequence selected from the group consisting of: SEQ ID NO: 8, SEQ ID NO: 12, SEQ ID NO: 16, SEQ ID NO: 41, SEQ ID NO: 44, and SEQ ID NO:45.

88. The method of claim 87, wherein the VL of the anti-Abeta antibody or antigen-binding fragment thereof comprises an amino acid sequence selected from the group consisting of: SEQ ID NO: 8, SEQ ID NO: 12, SEQ ID NO: 16, SEQ ID NO: 41, SEQ ID NO: 44, and SEQ ID NO:45.

89. The method of claim 84, wherein the VH and VL of the anti-Abeta antibody or antigen-binding fragment thereof comprise, respectively, amino acid sequences at least 90% identical to reference amino acid sequences selected from the group consisting of: SEQ ID NO: 4 and SEQ ID NO: 8; SEQ ID NO: 6 and SEQ ID NO: 8; SEQ ID NO: 10 and SEQ ID NO: 12; SEQ ID NO: 14 and SEQ ID NO: 16; SEQ ID NO: 39 and SEQ ID NO: 41; SEQ ID NO: 42 and SEQ ID NO: 44; and SEQ ID NO: 43 and SEQ ID NO: 45.

90. The method of claim 89, wherein the VH and VL of the anti-Abeta antibody or antigen-binding fragment thereof comprise, respectively, amino acid sequences selected from the group consisting of: SEQ ID NO: 4 and SEQ ID NO: 8; SEQ NO: 6 and SEQ ID NO: 8; SEQ ID NO: 10 and SEQ ID NO: 12; SEQ ID NO: 14 and SEQ ID NO: 16; SEQ ID NO: 39 and SEQ ID NO: 41; SEQ ID NO: 42 and SEQ ID NO: 44; and SEQ ID NO: 43 and SEQ ID NO: 45.

91. The method of claim 84, wherein the VH of the anti-Abeta antibody or antigen-binding fragment thereof comprises a Kabat heavy chain complementarity determining region-1 (VH-CDR1) amino acid sequence identical, except for two or fewer amino acid substitutions, to a reference VH-CDR1 amino acid sequence selected from the group consisting of: SEQ ID NO: 17, SEQ ID NO: 20, SEQ ID NO: 26, and SEQ ID NO: 32.

92. The method of claim 84, wherein the VH of the anti-Abeta antibody or antigen-binding fragment thereof comprises a Kabat heavy chain complementarity determining region-2 (VH-CDR2) amino acid sequence identical, except for four or fewer amino acid substitutions, to a reference VH-CDR2 amino acid sequence selected from the group consisting of: SEQ ID NO: 18, SEQ ID NO: 21, SEQ ID NO: 27, and SEQ ID NO: 33.

93. The method of claim 84, wherein the VH of the anti-Abeta antibody or antigen-binding fragment thereof comprises a Kabat heavy chain complementarity determining region-3 (VH-CDR3) amino acid sequence identical, except for four or fewer amino acid substitutions, to a reference VH-CDR3 amino acid sequence selected from the group consisting of SEQ ID NO: 19, SEQ ID NO: 22, SEQ ID NO: 28, and SEQ ID NO: 34.

94. The method of claim 84, wherein the VL of the anti-Abeta antibody or antigen-binding fragment thereof comprises a Kabat light chain complementarity determining region-1 (VL-CDR1) amino acid sequence identical, except for four or fewer amino acid substitutions, to a reference VL-CDR1 amino acid sequence selected from the group consisting of: SEQ ID NO: 23, SEQ ID NO: 29, SEQ ID NO: 35, SEQ ID NO: 46, and SEQ ID NO: 49.

95. The method of claim 84, wherein the VL of the anti-Abeta antibody or antigen-binding fragment thereof comprises a Kabat light chain complementarity determining region-2 (VL-CDR2) amino acid sequence identical, except for two or fewer amino acid substitutions, to a reference VL-CDR2 amino acid sequence selected from the group consisting of: SEQ ID NO: 24, SEQ ID NO: 30, SEQ ID NO: 36, SEQ ID NO: 47, and SEQ ID NO: 50.

96. The method of claim 84, wherein the VL of the anti-Abeta antibody or antigen-binding fragment thereof comprises a Kabat light chain complementarity determining region-3 (VL-CDR3) amino acid sequence identical, except for four or fewer amino acid substitutions, to a reference VL-CDR3 amino acid sequence selected from the group consisting of: SEQ ID NO: 25, SEQ ID NO: 31, SEQ ID NO: 37, SEQ ID NO: 48, and SEQ ID NO: 51.

97. The method of claim 84, wherein the VH of the anti-Abeta antibody or antigen-binding fragment thereof comprises VH-CDR1, VH-CDR2, and VH-CDR3 amino acid sequences selected from the group consisting of: SEQ ID NOs: 17, 18, and 19; SEQ ID NOs: 20, 21, and 22; SEQ ID NOs: 26, 27, and 28; and SEQ ID NOs: 32, 33, and 34.

98. The method of claim 84, wherein the VL of the anti-Abeta antibody or antigen-binding fragment thereof comprises VL-CDR1, VL-CDR2, and VL-CDR3 amino acid sequences selected from the group consisting of: SEQ ID NOs: 23, 24, and 25; SEQ ID NOs: 29, 30, and 31; SEQ ID NOs: 35, 36, and 37; SEQ ID NOs: 46, 47 and 48; and SEQ ID NOs 49, 50 and 51.

99. The method of claim 84, wherein the VH and VL are from a monoclonal antibody selected from the group consisting of NI-101.10, NI-101.11, NI-101.12, NI-101.13, NI-101.12F6A, NI-101.13A, and NI-101.13B.

100. The method of claim 84, wherein the anti-Abeta antibody or antigen-binding fragment thereof comprises a heavy chain constant region or fragment thereof.

101. The method of claim 100, wherein the heavy chain constant region or fragment thereof is human IgG1 or mouse IgG2A.

102. The method of claim 84, wherein the anti-Abeta antibody or antigen-binding fragment thereof further comprises a heterologous polypeptide fused thereto.

103. The method of claim 84, wherein the anti-Abeta antibody or antigen-binding fragment thereof is conjugated to an agent selected from the group consisting of a cytotoxic agent, a therapeutic agent, a cytostatic agent, a biological toxin, a prodrug, a peptide, a protein, an enzyme, a virus, a lipid, a biological response modifier, a pharmaceutical agent, a lymphokine, a heterologous antibody or fragment thereof, a detectable label, polyethylene glycol (PEG), and a combination of two or more of any of the agents.

104. The method of claim 103, wherein the cytotoxic agent is selected from the group consisting of a radionuclide, a biotoxin, an enzymatically active toxin, a cytostatic or cytotoxic therapeutic agent, a prodrug, an immunologically active ligand, a biological response modifier, or a combination of two or more of any of the cytotoxic agents.

105. The method of claim 103, wherein the detectable label is selected from the group consisting of an enzyme, a fluorescent label, a chemiluminescent label, a bioluminescent label, a radioactive label, or a combination of two or more of any of the detectable labels.

106. The method of claim 82, wherein the accumulation of Abeta is associated with a neurological disease, disorder, injury, or condition.

107. The method of claim 77, wherein the abnormal amyloid condition is associated with a neurological disease, disorder, injury, or condition.

108. The method of claim 107, wherein the disease, disorder, injury, or condition selected from the group consisting of Alzheimer's disease, Down's Syndrome, head trauma, dementia pugilistica, chronic traumatic encephalopathy (CTE), chronic boxer's encephalopathy, traumatic boxer's encephalopathy, boxer's dementia, punch-drunk syndrome, amyloid deposition associated with aging, mild cognitive impairment, cerebral amyloid angiopathy, Lewy body dementia, vascular dementia, mixed dementia, multi-facet dementia, hereditary cerebral hemorrhage with amyloidosis Dutch type and Icelandic type, glaucoma, Parkinson's disease, Huntington's disease, Creutzfeldt-Jakob disease, cystic fibrosis, or Gaucher's disease and inclusion body myositis.

109. The method of claim 107, wherein the disease, disorder, injury, or condition is Alzheimer's disease.

110. The method of claim 76, wherein the subject is a human.

111. The method of claim 76, wherein the Abeta binding molecule is administered intravenously, intramuscularly, subcutaneously, intraperitoneally, intranasally, parenterally or as an aerosol.
